(19) United States
(12) Patent Application Publication
(10) Pub. No.: US 2020/0132604 A1
(43) Pub. Date: Apr. 30, 2020

(54) METHODS OF DETERMINING PROTEIN STRUCTURE USING TWO-PHOTON FLUORESCENCE MEASUREMENTS

(71) Applicant: Biodesy, Inc., South San Francisco, CA (US)

(72) Inventors: Bason Clancy, Redwood City, CA (US); Joshua Salafsky, San Francisco, CA (US)

(21) Appl. No.: 16/668,279

(22) Filed: Oct. 30, 2019

Related U.S. Application Data
(63) Continuation of application No. PCT/US18/29234, filed on Apr. 24, 2018.
(60) Provisional application No. 62/500,912, filed on May 3, 2017.

(51) Int. Cl. G01N 21/64; G01N 33/68; G01N 33/566

(52) U.S. Cl. CPC G01N 21/6428; G01N 33/6803; G01N 2021/6439; G01N 21/6456; G01N 33/566

(57) ABSTRACT

Methods, devices, and systems for using two-photon fluorescence measurements, either alone or in combination with other nonlinear optical measurements such as second harmonic generation, sum frequency generation, or difference frequency generation, to determine structural parameters such as mean tilt angle and distribution width for tethered nonlinear-active biomolecules are described. The disclosed methods, devices, and systems may also be used to perform structural comparisons of two or more biomolecular samples; to detect changes in biomolecule conformation upon binding of a ligand; and to screen candidate binding partners to identify compounds that modulate the conformation of the biomolecule.

### CROSS-REFERENCE

This application is a Continuation Application which claims the benefit of International Patent Application No. PCT/US2018/029234, filed on Apr. 24, 2018, which claims the benefit of U.S. Provisional Application No. 62/500,912, filed on May 3, 2017, each of which is incorporated herein by reference in its entirety.

### BACKGROUND

The disclosed invention relates to the field of molecular detection, and in particular to the field of protein detection and structure determination. Although the field of protein structure determination (and more generally, biomolecular structure determination) is highly developed, there remains a need for sensitive and rapid techniques for determination of protein structure, comparison of protein structures from different samples or at different points in time, and detection of protein conformational changes in real time and in solution. Most information about protein structure and dynamics has come mainly from X-ray crystallography and NMR studies, but these techniques are relatively labor and material intensive, slow to perform, or provide only a static snapshot of protein structure.

Second harmonic generation (SHG) is a nonlinear optical process which may be configured as a surface-selective detection technique that enables detection of binding interactions and conformational change in proteins and other biological targets labeled with second harmonic-active labels (see, for example, U.S. Pat. Nos. 6,953,694, and 8,497,073). To date these methods have been applied to detect ligand-induced conformational changes in a variety of biological systems and to distinguish ligands by the type of conformational change they induce upon binding to target proteins (Salafsky, J. S. (2001), “'SHG-labels' for Detection of Molecules by Second Harmonic Generation”, Chemical Physics Letters 342, 485-491; Salafsky, J. S. (2003), “Second-Harmonic Generation as a Probe of Conformational Change in Molecules”, Chemical Physics Letters 381, 705-709; Salafsky, J. S. (2006), “Detection of Protein Conformational Change by Optical Second-Harmonic Generation”, Journal of Chemical Physics 125; Moree, B., et al. (2015), “Small Molecules Detected by Second Harmonic Generation Modulate the Conformation of Monomeric α-Synuclein and Reduce Its Aggregation in Cells”, J. Biol. Chem. 290(46);27582-27593; Moree, et al. (2015), “Protein Conformational Changes are Detected and Resolved Site Specifically by Second-Harmonic Generation”, Biophys. J. 109:806-815). Examples of the use of SHG for distinguishing between different types of ligands include distinguishing between type I vs. type II kinase inhibitors, such as imatinib and dasatinib, which bind to the protein to induce inactive and active conformations, respectively.

As described in the present disclosure, two-photon fluorescence (a different nonlinear optical technique) can also be used, either alone or in combination with nonlinear optical techniques such as second harmonic generation (SHG), sum frequency generation (SFG), or difference frequency generation (DFG), to address problems in structural biology and high throughput screening that include, but are not limited to, biomolecule structure determination in solution, at high throughput, and optionally, in real time; conformational landscape mapping; identification of false positives and false negatives in screening applications; and so forth. Accordingly, a first aspect of the present disclosure describes methods, devices, and systems for determining protein structure using two-photon fluorescence (TPF) measurements or combinations of two-photon fluorescence and other nonlinear optical measurements.

In another aspect of the present disclosure, methods, devices, and systems are described for using SHG and related nonlinear optical techniques to determine the absolute orientation of a label attached to a protein, thereby allowing one to map protein structure by systematically changing the attachment site of the label to the protein, or to compare protein structure between different samples or for a given sample at different points in time. In some embodiments, these measurements may comprise measuring SHG signals, e.g., baseline SHG signals, using polarized light. In some embodiments, these measurements may comprise measuring SHG-to-TPF signal ratios, e.g., baseline SHG-to-TPF signal ratios, or other ratios of nonlinear optical signals.

The disclosed methods, devices, and systems may have utility in a variety of fields, including basic research fields such as structural biology and drug discovery and development. In many cases, this information may not be available without employing laborious, time-intensive, and costly techniques such as x-ray crystallography.

### SUMMARY

Disclosed herein are methods for determining angular parameters of a two-photon fluorescent label attached to a tethered biomolecule, the method comprising: (a) attaching a biomolecule to a planar surface in an oriented manner, wherein the biomolecule is labeled at a known site with a two-photon fluorescent label; (b) illuminating the attached biomolecule with excitation light of a first fundamental frequency using a first polarization; (c) detecting a first physical property of light generated by the two-photon fluorescent label as a result of the illumination in step (b); (d) illuminating the attached biomolecule with excitation light of the first fundamental frequency using a second polarization; (e) detecting a second physical property of light generated by the two-photon fluorescent label as a result of the illumination in step (d); and (f) comparing the second physical property of light detected in step (e) to the first physical property of light detected in step (c) to determine angular parameters of the two-photon fluorescent label relative to the planar surface.

In some embodiments, the first physical property is p-polarized light intensity Iₚ and the second physical property is s-polarized intensity Iₛ and the comparison in step (f) comprises solving the following equation to determine angular parameters:

$$\frac{〈 {cos}^{4} {φsin}^{2} φ 〉}{〈 {sin}^{6} φ 〉} = \frac{3}{8} \frac{1}{{f}^{4}} \frac{{I}_{p}}{{I}_{s}} .$$

In some embodiments, the method further comprises repeating steps (a) through (f) for each of a series of two or more different biomolecule conjugates, wherein each of the biomolecule conjugates in the series comprises the biomolecule labeled at a different site with the same two-photon fluorescent label, and determining a structure of the biomolecule using the angular parameters determined for each of the two or more different biomolecule conjugates. In some embodiments, the biomolecule is a protein, and wherein the series of two or more different biomolecule conjugates each comprise a single-site cysteine or methionine substitution. In some embodiments, the biomolecule is labeled with two or more different two-photon fluorescent labels at two or more different sites, wherein a first physical property and a second physical property of light generated by each of the two or more different two-photon fluorescent labels are simultaneously or sequentially detected in steps (c) and (e) upon illumination by light of a fundamental frequency that may be the same or different for the two or more different two-photon fluorescent labels, and wherein a comparison of the second physical property of the light detected in step (e) to the first physical property of the light detected in step (c) for each of the two or more different two-photon fluorescent labels is used determine angular parameters of each of the two or more different two-photon fluorescent labels relative to the planar surface. In some embodiments, the attached biomolecule is also labeled at a known site with a second harmonic (SH)-active, sum frequency (SF)-active, or difference frequency (DF)-active label. In some embodiments, the two-photon fluorescent label and the first second harmonic (SH)-active, sum frequency (SF)-active, or difference frequency (DF)-active label are the same label attached to the same known site on the biomolecule. In some embodiments, the method further comprises simultaneously or subsequently detecting a first physical property of light generated by the second harmonic (SH)-active, sum frequency (SF)-active, or difference frequency (DF)-active label in step (c), and a second physical property of light generated by the second harmonic (SH)-active, sum frequency (SF)-active, or difference frequency (DF)-active label in step (e), upon illumination by excitation light of a second fundamental frequency, where the second fundamental frequency may be the same as or different from the first fundamental frequency. In some embodiments, the method further comprises comparing the second physical property of the light detected in step (e) to the first physical property of the light detected in step (c) to determine angular parameters of the second harmonic (SH)-active, sum frequency (SF)-active, or difference frequency (DF)-active label relative to the planar surface. In some embodiments, the method further comprises globally fitting data for the angular parameters of one or more two-photon fluorescent labels, second harmonic (SH)-active labels, sum frequency (SF)-active labels, or difference frequency (DF)-active labels, or any combination thereof, to a structural model of the biomolecule, wherein the structural model comprises information about the known sites of the one or more labels within the biomolecule. In some embodiments, the method further comprises incorporating x-ray crystallographic data, NMR data, or other experimental data which provide structural constraints for structural modeling of the biomolecule. In some embodiments, the biomolecule is a protein, and the two-photon fluorescent label or second harmonic (SH)-active, sum frequency (SF)-active, or difference frequency (DF)-active label is a nonlinear-active unnatural amino acid. In some embodiments, the nonlinear-active unnatural amino acid is L-Anap, Aladan, or a derivative of naphthalene. In some embodiments, a nonlinear-active moiety is attached to an unnatural amino acid that is not appreciably nonlinear-active. In some embodiments, the second physical property of light is different from the first physical property of light. In some embodiments, the first and the second physical properties of light possess the same polarization but are of different magnitudes or intensities. In some embodiments, the first and the second physical properties of light possess different polarizations. In some embodiments, the illuminating steps comprise adjusting the polarization of the excitation light. In some embodiments, a first polarization state of the excitation light comprises p-polarization relative to its plane of incidence, and a second polarization state of the excitation light comprises s-polarization relative to its plane of incidence. In some embodiments, the detecting in steps (c) and (e) comprises adjusting the polarization of the light generated by the two-photon fluorescent label or a second harmonic (SH)-active, sum frequency (SF)-active, or difference frequency (DF)-active label that reaches a detector. In some embodiments, the first and second physical properties of light are an intensity or a polarization. In some embodiments, the light generated by the two-photon fluorescent label is detected using a low numerical aperture pinhole configuration without the use of a collection lens. In some embodiments, the low numerical aperture pinhole is placed directly above or below a point on the planar surface at which the excitation light is incident on the planar surface. In some embodiments, the planar surface comprises a supported lipid bilayer and the biomolecules are attached to or inserted into the supported lipid bilayer. In some embodiments, the excitation light is directed to the planar surface using total internal reflection. In some embodiments, the two-photon fluorescent label is also second harmonic (SH)-active, sum frequency (SF)-active, or difference frequency (DF)-active, and further comprising determining angular parameters of the label by: (g) simultaneously or sequentially detecting an intensity of light generated by the second harmonic (SH)-active, sum frequency (SF)-active, or difference frequency (DF)-active label attached to the attached biomolecule upon illumination with excitation light of a second fundamental frequency which may be the same as or different from the excitation light of the first fundamental frequency, and wherein detection is performed using: (i) a first polarization state of the excitation light; and (ii) a second polarization state of the excitation light; (h) determining angular parameters of a second harmonic (SH)-active, sum frequency (SF)-active, or difference frequency (DF)-active label relative to a normal to the substrate surface by calculating a ratio of the light intensities detected in step (c)(i) and (c)(ii); (i) integrating an equation that relates angular parameters of the two-photon fluorescent label, and the light intensity ratio calculated for two-photon fluorescence to determine pairs of angular parameter values that satisfy the two-photon fluorescence equation; (j) integrating an equation that relates angular parameters of the second harmonic (SH)-active, sum frequency (SF)-active, or difference frequency (DF)-active label, and the light intensity ratio calculated for the second harmonic (SH), sum frequency (SF), or difference frequency (DF) light to determine pairs of angular parameter values that satisfy the second harmonic (SH), sum frequency (SF), or difference frequency equation; and (k) determining the intersection of the pairs of angular parameter values identified in steps (i) and (j) to determine a unique pair of angular parameter values that satisfy both the two-photon fluorescence and the second harmonic (SH), sum frequency (SF), or difference frequency equations. In some embodiments, biomolecules are attached to the planar surface such that the width of the orientational distribution of the two-photon fluorescent label attached to the biomolecule is less than or equal to 35 degrees. In some embodiments, the angular parameters comprise a mean tilt angle, an orientational distribution width, or a pairwise combination thereof.

Also disclosed herein are methods for detecting a conformational change in a biomolecule, the method comprising: a) attaching the biomolecule to a planar surface in an oriented manner, wherein the biomolecule is labeled with a two-photon fluorescent label; b) illuminating the attached biomolecule with excitation light of a first fundamental frequency using a first polarization and a second polarization; c) detecting a first physical property of light and a second physical property of light generated by the two-photon fluorescent label as a result of the illumination with the first and second polarizations in step (b); d) subjecting the attached biomolecule to (i) contact with a known ligand, (ii) contact with a candidate binding partner, or (iii) a change in experimental conditions; e) illuminating the attached biomolecule with excitation light of the first fundamental frequency using the first polarization and the second polarization; f) detecting a third physical property of light and a fourth physical property of light generated by the two-photon fluorescent label as a result of the illumination with the first and second polarizations in step (e); and (f) comparing a ratio of the third and fourth physical properties of light detected in step (f) to a ratio of the first and second physical properties of light detected in step (c), wherein a change in the ratio of physical properties of light indicates that the biomolecule has undergone a conformational change.

In some embodiments, the physical properties of two-photon fluorescent light are detected using a pinhole detection apparatus having a numerical aperture of less than or equal to 0.2. In some embodiments, the numerical aperture is between about 0.01 and about 0.2. In some embodiments, the physical properties of two-photon fluorescent light are detected without the use of a lens. In some embodiments, the two-photon fluorescent label is also second harmonic, sum frequency, or difference frequency active, and wherein a physical property of second harmonic, sum frequency, or difference frequency light is detected serially or simultaneously with the detection of the physical properties of the two-photon fluorescence as a result of illumination with light of a second fundamental frequency using the first and second polarizations. In some embodiments, the ratios compared in step (f) comprise ratios of the physical properties of two-photon fluorescence to the physical properties of second harmonic, sum frequency, or difference frequency light. In some embodiments, the second fundamental frequency is the same as the first fundamental frequency. In some embodiments, the first and second polarizations comprise s-polarization and p-polarization. In some embodiments, the biomolecule is a protein molecule. In some embodiments, the protein molecule is a drug target. In some embodiments, the known ligand is a known drug or the candidate binding partners are drug candidates. In some embodiments, the two-photon fluorescent label is attached to the protein molecule at one or more engineered cysteine residues. In some embodiments, the two-photon fluorescent label is pyridyloxazole (PyMPO). In some embodiments, the two-photon fluorescent label is a nonlinear-active unnatural amino acid that has been incorporated into the protein molecule. In some embodiments, the nonlinear unnatural amino acid is L-Anap, Aladan, or a derivative of naphthalene. In some embodiments, the excitation light is delivered to the planar surface using total internal reflection. In some embodiments, the biomolecule is attached to the planar surface by insertion into or tethering to a supported lipid bilayer.

Disclosed herein are methods for screening candidate binding partners to identify binding partners that modulate the conformation of a target molecule, the method comprising: (a) tethering the target molecule to a substrate surface, wherein the target molecule is labeled with a two-photon fluorescent label that is attached to a part of the target molecule that undergoes a conformational change upon contact with a binding partner, and wherein the tethered target molecule has a net orientation on the substrate surface; (b) illuminating the tethered target molecule with excitation light of a first fundamental frequency; (c) detecting a first physical property of light generated by the two-photon fluorescent label to generate a baseline signal; (d) sequentially and individually contacting the tethered target molecule with the one or more candidate binding partners; (e) detecting a second physical property of light generated by the two-photon fluorescent label in response to illumination by the excitation light of the first fundamental frequency for each of the one or more candidate binding partners; and (f) comparing the second physical property for each of the one or more candidate binding partners to the first physical property, wherein a change in value of the second physical property for a given candidate binding partner relative to that of the first physical property indicates that the candidate binding partner modulates the conformation of the target molecule.

In some embodiments, the first and second physical properties of light comprise the intensities of light under two different polarizations of the excitation light, and wherein step (f) comprises determining the ratio of the two intensities of light, wherein a change in the ratio indicates that the candidate binding partner modulates the conformation of the target molecule. In some embodiments, the target molecule is also labeled with a second harmonic (SH)-active, sum frequency (SF)-active, or difference frequency (DF)-active label. In some embodiments, the two-photon fluorescent label and the second harmonic (SH)-active, sum frequency (SF)-active, or difference frequency (DF)-active label are the same label moiety. In some embodiments, the method further comprises the steps of: (g) simultaneously with or subsequently to performing step (c), detecting a first physical property of light generated by the second harmonic (SH)-active, sum frequency (SF)-active, or difference frequency (DF)-active label upon illumination with excitation light of a second fundamental frequency, wherein the second fundamental frequency may be the same as or different than the first fundamental frequency; (h) simultaneously with or subsequently to performing step (e), detecting a second physical property of light generated by the second harmonic (SH)-active, sum frequency (SF)-active, or difference frequency (DF)-active label upon illumination with excitation light of the second fundamental frequency; and (i) comparing the second physical property generated by the second harmonic (SH)-active, sum frequency (SF)-active, or difference frequency (DF)-active label for each of the one or more candidate binding partners to the first physical property generated by the second harmonic (SH)-active, sum frequency (SF)-active, or difference frequency (DF)-active label, wherein a change in value of the second physical property for a given candidate binding partner relative to that of the first physical property further indicates that the candidate binding partner modulates the conformation of the target molecule. In some embodiments, the first and second physical properties of light comprise the intensities of light under two different polarizations of the excitation light, and wherein step (i) comprises determining the ratio of the two intensities of light, wherein a change in the ratio indicates that the candidate binding partner modulates the conformation of the target molecule. In some embodiments, the excitation light is directed to the substrate surface in such a way that it is totally internally reflected from the surface. In some embodiments, two-photon fluorescence is collected using a pin-hole aperture positioned directly above or below the substrate surface at a point where the excitation light of the first fundamental frequency is incident on the substrate surface. In some embodiments, two-photon fluorescence is collected without the use of a collection lens. In some embodiments, the numerical aperture of the pin-hole aperture is between 0.01 and 0.2. In some embodiments, the nonlinear-active label comprises a pyridyloxazole (PyMPO) moiety, a 6-bromoacetyl-2-dimethylaminonaphthalene (Badan) moiety, or a 6-Acryloyl-2-dimethylaminonaphthalene (Acrylodan) moiety. In some embodiments, the target molecule is a protein that comprises a genetically-incorporated His tag. In some embodiments, the His tag comprises a 6×-His tag, a 7×-His tag, an 8×-His tag, a 9×-His tag, a 10×-His tag, an 11×-His tag, or a 12×-His tag. In some embodiments, the tethered target molecule is illuminated with light of the first fundamental frequency through the use of total internal reflection.

Disclosed herein are methods for comparing the conformational changes induced by a generic drug or drug candidate and a reference drug in the structure of a target protein, wherein the target protein is labeled with a nonlinear-active label and is tethered to an interface such that is has a net orientation on the interface, the method comprising: a) contacting the target protein with the reference drug, wherein the target protein interacts with the reference or branded drug in a specific manner; b) detecting an interaction between the target protein and the reference drug by measuring a first signal or signal change generated by the nonlinear-active label using a surface-selective technique, wherein the first signal or signal change indicates a conformational change in the structure of the target protein that is specific for the reference drug; c) contacting the target protein with the generic drug or drug candidate, wherein the target protein interacts with the generic drug or drug candidate in a specific manner; and d) detecting an interaction between the target protein and the generic drug or drug candidate by measuring a second signal or signal change generated by the nonlinear-active label using a surface-selective technique, wherein the second signal or signal change indicates a conformational change in the structure of the target protein that is specific for the generic drug or drug candidate; and e) comparing the second signal or signal change to the first signal or signal change to determine whether the conformational change induced in the target protein by the generic drug or drug candidate is the same or substantially the same as the change induced by the reference drug.

In some embodiments, the target protein is a cell surface receptor or an antigen. In some embodiments, the reference drug is a monoclonal antibody (mAb). In some embodiments, the generic drug or candidate drug are selected from the group consisting of a small molecule chemical compounds, a non-antibody inhibitory peptide, an antibody, and any combination thereof. In some embodiments, the generic drug or drug candidate is a monoclonal antibody (mAb). In some embodiments, the generic drug is a biosimilar. In some embodiments, the conformational change in the structure of the target protein is detected in real time. In some embodiments, the nonlinear-active label is bound to the target protein by one or more sulthydryl groups on the surface of the target protein. In some embodiments, the said one or more sulthydryl groups are engineered sulthydryl groups. In some embodiments, the nonlinear-active label is a second harmonic (SH)-active label or a two-photon fluorescent label. In some embodiments, the nonlinear-active label is a second harmonic (SH)-active label selected from the group consisting of PyMPO maleimide, PyMPO-NHS, PyMPO succinimidyl ester, Badan, and Acrylodan. In some embodiments, the nonlinear-active label is an unnatural amino acid. In some embodiments, the unnatural amino acid is L-Anap, Aladan, or a derivative of naphthalene. In some embodiments, a determination of biosimilarity is made on the basis of the comparison of induced conformational changes in combination with structural or functional data obtained from at least a second structural characterization or functional assay technique. In some embodiments, the at least second structural characterization or functional assay technique is selected from the group consisting of circular dichroism, x-ray crystallography, biological assays, binding assays, enzymatic assays, cell-based assays, cell proliferation assays, cell-based reporter assays, and animal model studies.

Disclosed herein are methods for comparing two or more protein samples, the method comprising: a) providing two or more protein samples collected at different times for the same step of a protein production process, at different steps of a protein production process, from separate runs of the same protein production process, or from different protein production processes that nominally produce the same protein; b) tethering the protein from the one or more protein samples in one or more discrete regions of an optical interface, wherein the tethered protein from each sample is labeled with a nonlinear-active label and has a net orientation at the optical interface; c) measuring a baseline nonlinear optical signal for each of the one or more tethered protein samples that is generated upon illumination of the nonlinear active label with light of a fundamental frequency; and d) comparing the measured baseline nonlinear optical signals for the one or more tethered protein samples with each other or with a baseline nonlinear optical signal measured for a reference sample, wherein a difference in the baseline nonlinear optical signals measured for the one or more immobilized protein samples, or between the baseline nonlinear optical signals measured for the one or more protein samples and that of a reference sample, of less than a specified percentage indicates that the proteins of the one or more protein samples or the reference sample have equivalent structures.

In some embodiments, the one or more protein samples are collected at an endpoint of a protein production process, and the comparison in step (d) is used for quality control of the protein product. In some embodiments, the one or more protein samples are collected at one or more steps of a protein production process, and the comparison in step (d) is used for optimization of the protein production process. In some embodiments, the one or more protein samples are collected from different protein production processes that nominally produce the same protein, and the comparison in step (d) is used to demonstrate biosimilarity. In some embodiments, the optical interface comprises a surface selected from the group consisting of a glass surface, a fused-silica surface, or a polymer surface. In some embodiments, the optical interface comprises a supported lipid bilayer. In some embodiments, the supported lipid bilayer further comprises Ni/NTA-lipid molecules. In some embodiments, the proteins of the one or more protein samples comprise a His-tag. In some embodiments, the baseline nonlinear optical signal, or changes thereof, is monitored in real time. In some embodiments, the nonlinear-active label is bound to the protein by one or more sulfhydryl groups on the surface of the protein. In some embodiments, the said one or more sulfhydryl groups are engineered sulfhydryl groups. In some embodiments, the nonlinear-active label is a second harmonic (SH)-active label. In some embodiments, the immobilized or tethered protein is labeled by contacting it with peptide, peptidomimetic, or other ligand that itself is SHG-active, resulting in the SHG-active ligand bound to the immobilized or tethered protein. In some embodiments, the nonlinear-active label is a second harmonic (SH)-active label selected from the group consisting of PyMPO maleimide, PyMPO-NHS, PyMPO succinimidyl ester, Badan, and Acrylodan. In some embodiments, the nonlinear-active label is an unnatural amino acid that has been genetically-incorporated into the proteins of the one or more protein samples. In some embodiments, the unnatural amino acid is L-Anap, Aladan, or a derivative of naphthalene. In some embodiments, the nonlinear active label is both second harmonic (SH)-active and two-photon fluorescent, and wherein the measuring in step (c) further comprises measuring both a baseline second harmonic signal and a baseline two-photon fluorescence signal. In some embodiments, the comparison of step (d) further comprises comparing a ratio of second harmonic to two-photon fluorescence baseline signals for the one or more tethered protein samples with each other of with that for a reference sample, wherein a difference of less than a specified percentage indicates that the proteins of the one or more protein samples or the reference sample have equivalent structures. Disclosed herein are methods for detecting two-photon fluorescence of a two-photon fluorescent label attached to a tethered biomolecule, the method comprising: (a) attaching a biomolecule to a planar surface in an oriented manner, wherein the biomolecule is labeled at a known site with a two-photon fluorescent label; (b) illuminating the attached biomolecule with excitation light of a fundamental frequency using a first polarization; (c) detecting a physical property of light generated by the two-photon fluorescent label as a result of the illumination in step (b), wherein the light generated by the two-photon fluorescent label is detected using a low numerical aperture pinhole configuration without the use of a collection lens. In some embodiments, the low numerical aperture pinhole is placed directly above or below a point on the planar surface at which the excitation light is incident on the planar surface. In some embodiments, the planar surface comprises a supported lipid bilayer and the biomolecules are attached to or inserted into the supported lipid bilayer. In some embodiments, the excitation light is directed to the planar surface using total internal reflection. In some embodiments, the low numerical aperture pinhole has a numerical aperture of between 0.01 and 0.2.

Also disclosed herein are methods for establishing the structural equivalence of a biosimilar drug candidate and a reference drug, the method comprising: a) labeling both the biosimilar drug candidate and the reference drug with a nonlinear-active label using an identical labeling reaction; b) tethering the nonlinear-active labeled biosimilar drug candidate and reference drug to an interface such that they have a net orientation on the interface; c) measuring a physical property of second harmonic light generated by the non-linear active label for both the biosimilar drug candidate and the reference drug upon illumination with light of a first fundamental frequency; d) optionally, measuring a physical property of two-photon fluorescence generated by the nonlinear-active label for both the biosimilar drug and the reference drug upon illumination with light of a second fundamental frequency; e) comparing the physical property of second harmonic light measured for the biosimilar drug candidate to that for the reference drug, wherein a statistically significant difference in the physical properties for the biosimilar drug candidate and the reference drug indicate that they are not structurally equivalent; and f) optionally, calculating a ratio of the physical property of second harmonic light to the physical property of two-photon fluorescence measured for the biosimilar drug candidate to that for the reference drug, wherein a statistically significant difference in the ratio calculated for the biosimilar drug candidate and the reference drug indicate that they are not structurally equivalent.

In some embodiments, the first fundamental frequency and the second fundamental frequency are the same. In some embodiments, the labeling reaction comprises covalent conjugation of the nonlinear-active label to a native functional group on the biosimilar drug candidate and reference drug. In some embodiments, the native functional group comprises a native amine group, a native carboxyl group, or a native sulfhydryl group. In some embodiments, the labeling reaction comprises covalent conjugation of the nonlinear-active label to a genetically-engineered functional group on the biosimilar drug candidate and reference drug. In some embodiments, the genetically-engineered functional group comprises a genetically-engineered amine group, a genetically-engineered carboxyl group, or a genetically-engineered sulfhydryl group. In some embodiments, the labeling reaction comprises a non-covalent interaction between a nonlinear-active labeled peptide that is known to bind to a specific region of the reference drug. In some embodiments, the nonlinear-active labeled peptide comprises a peptide known to bind to the FC region of a monoclonal antibody. In some embodiments, the nonlinear-active label comprises a pyridyloxazole (PyMPO) moiety, a 6-bromoacetyl-2-dimethylaminonaphthalene (Badan) moiety, or a 6-Acryloyl-2-dimethylaminonaphthalene (Acrylodan) moiety. In some embodiments, the nonlinear-active labeled biosimilar drug candidate and reference drug are tethered to the same interface. In some embodiments, the nonlinear-active labeled biosimilar drug candidate and reference drug are tethered to different interfaces. In some embodiments, the nonlinear-active labeled biosimilar drug candidate and reference drug are tethered to the interface using Protein A or Protein G molecules that are immobilized on the interface. In some embodiments, the interface comprises a supported lipid bilayer, and wherein the nonlinear-active labeled biosimilar drug candidate and reference drug are tethered to or embedded within the supported lipid bilayer. In some embodiments, the interface comprises a supported lipid bilayer, and wherein the nonlinear-active labeled biosimilar drug candidate and reference drug are tethered to the supported lipid bilayer using a genetically-incorporated His tag that binds to a bilayer lipid comprising a Ni-NTA moiety. In some embodiments, the genetically-incorporated His tag comprises a 6×-His tag, a 7×-His tag, an 8×-His tag, a 9×-His tag, a 10×-His tag, an 11×-His tag, or a 12×-His tag. In some embodiments, the nonlinear-active labeled biosimilar drug candidate and reference drug are illuminated with light of the first fundamental frequency through the use of total internal reflection. In some embodiments, two-photon fluorescence is collected using a pin-hole aperture positioned directly above or below the interface at a point where the excitation light of the first fundamental frequency is incident on the interface. In some embodiments, two-photon fluorescence is collected without the use of a collection lens. In some embodiments, a statistically significant difference in the physical property of light measured for the biosimilar drug candidate and reference drug, or in the ratio calculated for the biosimilar drug candidate and the reference drug, is indicated by a p-value of less than 0.05.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C provide schematic illustrations of the energy level diagrams for two-photon fluorescence (FIG. 1A), one-photon fluorescence (FIG. 1B), and second harmonic generation (FIG. 1C).

FIG. 2 illustrates the relationship between the laboratory frame of reference (as defined by X, Y, and Z axes) and the molecular frame of reference (as defined by X′, Y′, and Z′ axes). For some nonlinear-active molecules, the hyperpolarizability tensor (α⁽²⁾) may be dominated by a single component in the molecular frame of reference, i.e., α⁽²⁾=α⁽²⁾_{Z′,Z′,Z′}.

FIG. 3 illustrates a conformational change in a protein (labeled with a second harmonic-active label, a two-photon fluorescent label, or other nonlinear-active moiety) which is induced by binding of a ligand, and its impact on the orientation of the label relative to the Z-axis normal for an optical interface to which the protein is attached.

FIG. 4 provides a schematic illustration of one non-limiting example of a device comprising a patterned array of electrodes surrounding an area of a substrate surface used to form a supported lipid bilayer.

FIG. 5 provides a schematic illustration of one non-limiting example of a device for performing high-throughput structure determination using surface-selective nonlinear-optical techniques, wherein an array of hemispherical prisms bonded to or integrated with the substrate in a glass-bottom microplate format are used to provide good optical coupling of the excitation light to the top surface of the substrate.

FIG. 6 illustrates one non-limiting example of the system architecture for a high throughput analysis system for determining structure or conformational change of biological molecules, e.g., proteins or other biological entities, based on nonlinear optical detection.

FIG. 7 presents a schematic of a low-NA detection scheme where a 0.5 mm diameter fiber is used to collect the emitted fluorescence. In TPF, fluorescence is emitted in all directions (light shading), but a small segment of the total solid angle (dark shading) can be selected by using a geometry where the ratio of the fiber radius (0.5 mm) to the distance between the sample and the fiber (7.5 mm) is small, thereby producing a low-NA detector.

FIG. 8 shows a schematic for one non-limiting example of an optical setup used for analysis of structure or conformational change in biological molecules using nonlinear optical detection.

FIG. 9 shows a schematic illustration depicting the use of a prism to direct excitation light at an appropriate incident angle such that the excitation light undergoes total internal reflection at the top surface of a substrate. The two dashed lines to the right of the prism indicate the optical path of the reflected excitation light and the nonlinear optical signal generated at the substrate surface when nonlinear-active species are tethered to the surface. The substrate is optionally connected to the actuator of an X-Y translation stage for re-positioning between measurements. The curved lines between the top surface of the prism and the lower surface of the substrate indicate the presence a thin layer (not to scale) of index-matching fluid used to ensure high optical coupling efficiency between the prism and substrate.

FIGS. 10A-B illustrate a microwell plate with integrated prism array for providing good optical coupling of the excitation light to the top surface of the substrate. Such a device may be useful in conducting high-throughput structure determination of proteins and other biological molecules. FIG. 10A: top axonometric view. FIG. 10B: bottom axonometric view.

FIGS. 11A-B show exploded views of the microwell plate device shown in FIGS. 10A-B. FIG. 11A: top axonometric view. FIG. 11B: bottom axonometric view.

FIG. 12 illustrates the incident and exit light paths for coupling the excitation light to the substrate surface via total internal reflection using the design concept illustrated in FIGS. 11A-B.

FIG. 13 illustrates a computer system that may be configured to control the operation of the systems disclosed herein.

FIG. 14 is a block diagram illustrating a first example architecture of a computer system that can be used in connection with example embodiments of the present invention.

FIG. 15 is a diagram showing one embodiment of a network with a plurality of computer systems, a plurality of cell phones and personal data assistants, and Network Attached Storage (NAS).

FIG. 16 is a block diagram of a multiprocessor computer system using a shared virtual address memory space in accordance with an example embodiment.

FIGS. 17A-B provide non-limiting schematic illustrations of an optical setup used to make two-photon fluorescence (TPF) and second harmonic generation (SHG) measurements according to the disclosed methods. FIG. 17A: top view. FIG. 17B: side view.

FIG. 18 shows plots of mean orientational tilt angle versus the width of the orientational distribution for a M16C mutant of the protein dihydrofolate reductase (DHFR) tethered to a supported lipid bilayer, as determined by fitting two-photon fluorescence polarization intensity ratio measurements (green line) and SHG polarization intensity ratio measurements (blue line) assuming a Gaussian orientational distribution. The intersection of the two curves defines the unique angular parameters.

FIG. 19 shows plots of mean orientational tilt angle versus the width of the orientational distribution for a M16C mutant of the protein dihydrofolate reductase (DHFR) tethered to a supported lipid bilayer in the presence of 1 μM TMP, as determined by fitting two-photon fluorescence polarization intensity ratio measurements (green line) and SHG polarization intensity ratio measurements (blue line) assuming a Gaussian orientational distribution. The intersection of the two curves defines the unique angular parameters.

FIG. 20 displays angular data for the mean orientation and distribution width for all single-cysteine DHFR mutants used in Example 1. The orientational mean angle is plotted as a function of orientational distribution for each mutant with and without TMP demonstrating that the label adopts a large range of angles when placed at different locations throughout the protein.

FIG. 21 displays the change in orientational mean angle and change in orientational distribution for each single-cysteine mutant after addition of TMP.

FIG. 22 shows an overlay of two crystal structures of DHFR. The tan colored structure is the apo form, while the blue colored structure is bound with MTX. Residues that can be exchanged with cysteines are labeled in the figure and the projection of their side chains with respect to the peptide backbone is shown. The changes in side chain orientation observed between the two crystal structures can be compared to the changes in orientational mean angle and orientational distribution predicted by the described method.

### DETAILED DESCRIPTION

The methods, devices, and systems disclosed herein generally relate to the field of biomolecular detection and characterization using second harmonic generation (SHG), sum frequency generation (SFG), difference frequency generation (DFG), and/or two-photon fluorescence (TPF), including determination of biomolecular structure and dynamics. Methods for determining the relative and/or absolute orientation of nonlinear-active labels, e.g., SHG labels or TPF labels (also referred to as “probes”), attached to proteins or other biological molecules at one or more sites, and for determining molecular structures or conformations therefrom, are described. Also described are methods for comparison of protein or other biomolecular structures from different samples or for the same sample at different points in time.

In a first aspect of the present disclosure, methods, devices, and systems for determining protein structure using two-photon fluorescence (TPF) measurements, alone or in combination with SHG, SFG, or DFG measurements, are described. In general, the disclosed methods for determining structure or detecting conformational change in proteins or other biomolecules using TPF comprise: (a) attaching biomolecules to a planar surface, wherein the biomolecules are labeled at a known site with a two-photon fluorescent label; (b) illuminating the attached biomolecules with excitation light of a first fundamental frequency using a first polarization; (c) detecting a first physical property of light generated by the two-photon fluorescent label as a result of the illumination in step (b); (d) illuminating the attached biomolecules with excitation light of the first fundamental frequency using a second polarization; (e) detecting at least a second physical property of light generated by the two-photon fluorescent label as a result of the illumination in step (d); and (f) comparing the at least second physical property of light detected in step (e) to the first physical property of light detected in step (c) to determine an orientation of the two-photon fluorescent label relative to the surface plane. In a preferred embodiment of this method, the first and second physical properties of light are the TPF or SHG intensities measured with low numerical aperture (low NA) detection from single-site-specifically labeled biomolecules attached to a planar surface under p-polarized and s-polarized fundamental excitation light relative to the surface plane, respectively, and undergoing total internal reflection (TIR); and step (f) of this preferred embodiment comprises taking the ratio of the TPF or SHG intensities measured under p- and s-polarized excitation, e.g., R_{TPF}=Iₚ/Iₛ where R_{TPF} denotes the ratio of the intensities of TPF under p-polarized or s-polarized excitation.

Most biomolecules must be labeled with a nonlinear-active label to be rendered nonlinear-active themselves. For biomolecules such as proteins, the protein may be labeled covalently at, for example, one or more amine or sulfhydryl sites (e.g., one or more lysine or cysteine residues) with a two-photon fluorescence (TPF)-active probe, a second harmonic generation (SHG)-active probe, or optionally, with a probe which is both TPF-active and SHG-active, in order to confer nonlinear optical activity. Alternative labeling strategies may also be employed, as will be described in more detail below.

As noted above, in the methods of the present disclosure two-photon fluorescence (TPF) measurements may be used alone, or as an orthogonal or complementary approach to SHG measurement techniques, for determination of protein structure or detection of conformational change. Unlike SHG, TPF is not a coherent technique, and therefore does not require a net average orientation of labeled molecules in order to produce a signal. In some embodiments, ratios of nonlinear optical signal measurements, e.g., SHG-to-TPF signal ratios, may be utilized for protein structure determination or detection of conformational change.

In some embodiments, the illuminating (excitation) steps of the disclosed methods may comprise adjusting the polarization of excitation light of at least one fundamental frequency. In other embodiments, the frequency of the excitation light may be varied between experiments. In some embodiments, as will be discussed in more detail below, the excitation light used to perform TPF and/or SHG, SFG, or DFG measurements is directed to the surface in such a way that it is totally internally reflected from the surface. In some embodiments, a first polarization state of the excitation light comprises p-polarization relative to its plane of incidence, and a second polarization state of the excitation light comprises s-polarization relative to its plane of incidence.

In some embodiments, the determination of structural parameters, conformational state, and/or detection of conformational change in labeled biomolecules using TPF measurements (alone or in combination with SHG, SFG, or DFG measurements) comprises measuring a physical property, or a change in a physical property, of the nonlinear optical signal (e.g., a change in signal intensity or polarization) or a ratio, or a change in a ratio, of physical properties of nonlinear optical signals (e.g., a ratio of SHG-to-TPF signal intensities). In some embodiments, a first physical property of light is measured prior to contacting the labeled biomolecule with a ligand or subjecting it to some other environmental change, and at least a second physical property is measured after contacting the labeled biomolecule with the ligand or subjecting it to some other environmental change. In some embodiments, the at least second physical property of light is the same as the first physical property of light. In some embodiments, the at least second physical property of light is different from the first physical property of light. In some embodiments, multiple measurements may be made wherein the polarization, magnitude, or intensity, or any combination thereof, of the excitation light or the detected light is varied. In some embodiments, the methods further comprise incorporating x-ray crystallographic data, NMR data, or other experimental data which provide structural constraints for the protein into a structural model of the protein molecule (or other biomolecule).

A first key component of the TPF-based methods disclosed herein is that the TPF signals are produced using total internal reflection (TIR) excitation. TIR excitation has two important advantages: (i) it produces well determined orthogonal polarization states (p and s), and (ii) it generates TPF only in a thin evanescent region (˜100 nm) adjacent to the surface on which the labeled proteins are tethered. Both advantages lead to simplified theoretical analysis and significantly less error in calculating angular information about the probes (or labels), which is crucial for accurate structural measurements of labeled biomolecules such as proteins. The vast majority of TPF work described previously has employed epifluorescence microscopy, which involves excitation by a beam normal to the surface plane. Epifluorescence microscopy is convenient for imaging, but it leads to background TPF generation and to significant uncertainty in the analysis of tilt angles and other orientational information. For such structural analysis, it is required that one know with a high degree of confidence what the collection efficiency of the optical system is for the emitted photons. Moreover, it is not possible when using epifluorescence excitation to excite the tethered, labeled molecules with a polarization component in the z-direction (p-polarization).

A second key component of the TPF-based methods disclosed herein is that the two-photon fluorescence is collected without using a lens (unlike the case for microscopy) since that would require a detailed and precise knowledge of the lens numerical aperture (NA) in order to relate the measured fluorescence intensity to probe orientational distribution. Rather, the disclosed methods make use of a low-NA pinhole positioned either directly above or below the point at which excitation light is focused, and oriented in parallel to the sample plane (i.e., centered on the axis normal to the surface (z-axis) that passes through the focal point). Light passing through the pinhole may then be detected using a photomultiplier or other suitable detector. Accordingly, one of the main aims of the present invention is to enable high accuracy measurements of probe orientational distribution (and thereby infer information about protein structure and conformation) by employing TIR excitation and low-NA pinhole detection, where the pinhole is positioned directly above or below the sample at the point of excitation.

A third key component of the disclosed TPF-based methods is the use of a planar sample format in which the biomolecules are substantially confined to a single plane such as occurs with a monolayer, a supported lipid bilayer membrane, and so forth. This feature of the present invention both greatly simplifies the analysis and permits determination of orientational information such as average tilt angle and orientational width (e.g., assuming a Gaussian distribution) with significantly higher accuracy compared with prior art methods.

Finally, a fourth key component of the disclosed TPF-based methods, in particular for cases where angular information is desired, is the use of at least one TPF-active probe incorporated within the biomolecule of interest that is relatively narrowly distributed in its orientation relative to the surface, e.g., with a mean tilt angle having a standard deviation of less than or equal to approximately 35 degrees assuming a Gaussian distribution. As demonstrated in the examples below, we can achieve relatively narrow orientational distribution of the probes by tethering proteins via a 6× His-tag to a supported lipid bilayer comprising a capture agent, Ni-NTA lipid, e.g., 1,2-dioleoyl-3-glycero-3-[(N-(5-amino-1-carboxypentyl)iminodiacetic acid)succinyl] (nickel salt).

In some embodiments, structural determinations based on TPF measurements, alone or in combination with SHG, SFG, or DFG measurements, may be facilitated by performing the measurements under two or more different sets of experimental conditions. For example, in some embodiments, the protein (or other biomolecule) is attached to a surface or a supported lipid bilayer using a His-tag. In some embodiments, a first set of experimental conditions comprises tethering the protein molecules using a His-tag attached to the N-terminus of the protein, and an at least second set of experimental conditions comprises tethering the protein molecules using a His-tag attached to the C-terminus. Alternatively, in some embodiments, the first set of experimental conditions may comprise tethering the protein molecules in the presence of a first assay buffer (or exposing tethered proteins to a first assay buffer), and an at least second set of experimental conditions may comprise tethering the protein molecules in the presence of an at least second assay buffer (or exposing tethered proteins to an at least second assay buffer) that differs from the first assay buffer. In some embodiments, as noted above, the difference between the first set of experimental conditions and the at least second set of experimental conditions may comprise contacting the tethered protein molecules with at least a first ligand that is known to bind to and induce conformational change in the protein molecules. Non-limiting examples of different sets of experimental conditions that may be used to facilitate structural determinations based on TPF measurements, alone or in combination with SHG, SFG, or DFG measurements, will be described in more detail below. The aim of using different experimental conditions is to produce a sample in which the orientational distribution is varied in the lab frame, thus providing an independent set of angular measurements for a biomolecule labeled at a given site, i.e. with different projections of the probe transition dipole moment(s) on the surface normal axis (z-axis). This enables more equations for determining the conformational distribution (landscape) in the biomolecule's frame of reference.

In another aspect of the present disclosure, the use of SHG or related nonlinear optical baseline signals (e.g., SFG and DFG baseline signals) for the comparison of protein structures from different samples or from a given sample at different points in time is described. In general, these methods may comprise: (i) labeling the proteins in one or more samples with a nonlinear-active label using identical labeling reactions and reaction conditions, (ii) tethering the labeled proteins to an interface (e.g., a substrate surface) such that they have a net orientation on the interface, (iii) measuring a physical property of light generated by the nonlinear-active label upon illumination with light of a fundamental frequency, and (iv) comparing the nonlinear optical signals measured for different samples, or measured for a given sample at different points in time. Such baseline signal measurements may be performed, for example, to: (i) compare protein structure between different lots of purified protein, (ii) to monitor protein structural variation at different steps in bioreactor or manufacturing processes for expression, production, and/or purification of protein products, or (iii) to monitor protein stability upon contacting the protein with different reagents or subjecting it to different experimental conditions. The approach makes use of variations in baseline SHG or other nonlinear optical signals as a measure of the degree of denaturation of a protein that has been labeled with a nonlinear-active moiety. In some embodiments, these comparisons may rely solely on measurements of SHG, SFG, or DFG baseline signals. In preferred embodiments, these comparisons may be made using a ratio of the SHG, SFG, or DFG baseline signal to a TPF baseline signal measured for the same sample, where the SHG, SFG, or DFG baseline signal and the TPF baseline signal are measured simultaneously or serially. The use of SHG, SFG, or DFG baseline signal-to-TPF baseline signal ratios allows one to normalize the SHG, SFG, or DFG baseline signal and correct for variations in the surface density of labeled protein molecules tethered to a substrate that may exist from well to well or from experiment to experiment used to excite the nonlinear-active labels in a surface-selective manner. Such structural comparisons have potential utility in a variety of drug discovery and development applications (and other fields) including, but not limited to, monitoring of protein stability for biological drugs, manufacturing process monitoring and quality control, and demonstration of biosimilarity between biological drug candidates and reference drugs.

In addition to the disclosed nonlinear optical methods for determining biomolecular structure/conformation, and for comparing biomolecular structures for two or more different samples or for the same sample at different points in time, devices and systems are described which facilitate the performance of the disclosed methods and/or their implementation in a high throughput format for analysis of molecular orientation or molecular structure. In some aspects of the present disclosure, methods, devices, and systems are described for determining orientation, conformation, structure, or changes in orientation, conformation, or structure of biological molecules in response to contacting the biological molecules with one or more test molecules (e.g., known ligands, candidate binding partners, and/or drug candidates). In some aspects of the present disclosure, methods, devices, and systems are described for determining orientation, conformation, structure, or changes in orientation, conformation, or structure of biological molecules in response to subjecting the biological molecules to two or more different sets of experimental conditions. As used herein, determining biomolecule orientation, conformation, structure, or changes thereof, may involve measurement of at least one nonlinear optical signal which is proportional to the average orientation of a nonlinear-active label or tag, and which may also be proportional to the surface density of labeled biological molecules tethered to a surface. As used herein, “high throughput” refers to the ability to perform rapid analysis (relative to, for example, crystallographic structure determination) of molecular orientation, conformation, structure, or changes thereof for a plurality of biological molecules optionally contacted with one or more known ligands, candidate binding partners, and/or drug candidates, or to the ability to perform rapid analysis of molecular orientation, conformation, structure, or changes thereof for one or more biological molecules optionally contacted with a large plurality of known ligands, candidate binding partners, and/or drug candidates, or to any combination of these modalities.

Definitions: Unless otherwise defined, all technical terms used herein have the same meaning as commonly understood by one of ordinary skill in the art in the field to which this disclosure belongs. As used in this specification and the appended claims, the singular forms “a”, “an”, and “the” include plural references unless the context clearly dictates otherwise. Any reference to “or” herein is intended to encompass “and/or” unless otherwise stated.

Biological molecules: Although described primarily in the context of characterization of protein samples, those of skill in the art will recognize that the disclosed nonlinear optical methods may be advantageously utilized for structural and conformational characterization of a variety of other types of biomolecules. As used herein, the phrases “biological molecules”, “biomolecules”, or in some cases “biological entities” include, but are not limited to, proteins, protein domains or sub-domains, peptides, receptors, enzymes, antibodies, antibody fragments, DNA, RNA, oligonucleotides, DNA or RNA aptamers, small molecules, synthetic molecules, carbohydrates, or in some cases, cells, or any combination thereof. In some embodiments, biological molecules may comprise drug targets, or portions thereof, and may be referred to as “target proteins” or “target molecules”. In some preferred embodiments of the present disclosure, the target molecules are proteins, or subunits, subdomains, or fragments thereof. In some preferred embodiments, the target proteins are biological drug candidates (biosimilar drug candidates) and/or reference drugs

Test molecules: Similarly, the phrases “test molecules”, “test compounds”, “candidate binding partners”, “drug candidates”, or in some cases, “test entities”, include, but are not limited to, cells, proteins, peptides, receptors, enzymes, antibodies, DNA, RNA, DNA or RNA aptamers, biological molecules, oligonucleotides, buffers, solvents, small molecules, synthetic molecules, carbohydrates, or in some cases, cells, or any combination thereof. In some embodiments, test molecules may comprise known ligands, drug candidates, or portions thereof. In some preferred embodiments of the present disclosure, the drug candidates are other proteins, or subunits, subdomains, or fragments thereof. In some preferred embodiments, the drug candidates are biological drug candidates (e.g., biosimilar drug candidates).

Biologics: As used herein, the term “biologics” (also referred to as “biological products” or “biological therapeutics”) refers to products that are isolated from a variety of natural sources (e.g., human, animal, or microorganism) or that may be produced by genetic engineering and other biotechnology methods. Biologics may comprise sugars, proteins, protein fragments, nucleic acids, or complex combinations of these substances, or may comprise living entities such as cells (and tissues) that have clinical diagnostic or therapeutic application.

Biosimilars: As used herein, the term “biosimilar” (or biosimilar product”) refers to a biological product that is approved based on a showing that it is highly similar to a biological product that has received regulatory approval (known as a “reference product”), and has no clinically meaningful differences from the reference product in terms of safety and effectiveness. Thus, a biosimilar is a generic version of an existing biological drug. A biosimilar drug candidate (or biological drug candidate) is a biologic that has yet to be approved.

Reference drugs: As used herein, the term “reference drug” (or “reference product”) refers to an approved drug product (e.g., a small molecule drug or a biologic such as a therapeutic monoclonal antibody) to which new generic versions are compared to show that they are bioequivalent.

Reference samples: As used herein, the term “reference sample” refers to a protein (or other biomolecule) sample that has been prepared at a different point in time or that has been prepared using a protein from a different production process or production lot. Nominally, the protein to be studied and the protein of the reference sample have the same amino acid sequence.

Angular parameters: As used herein, the term “angular parameters” refers to a mean tilt angle of a probe relative to the surface normal, the orientational distribution width around the mean tilt angle, i.e., (ϕ, σ) as defined herein, a pairwise combination of mean tilt angle and orientational distribution width, ratios of intensities of TPF, SHG, DFG, SFG measured at two different polarizations (e.g., ratios of s- and p-polarized intensities), or any other angular parameter, intensity of light measured under excitation of light at a specific polarization, or combination of intensity measurements made at different frequencies, polarizations, or other physical properties of either detected or excitation light, known to those skilled in the art, to characterize angular parameters of the probe.

Nonlinear optical techniques: As used herein, the phrase “nonlinear optical technique” includes second harmonic generation, sum frequency generation, difference frequency generation, and/or two-photon fluorescence. Second harmonic generation is a nonlinear optical process wherein two photons of excitation light at a fundamental frequency interact with a nonlinear material or molecule and are re-emitted or scattered as a single photon having energy equal to twice that of the excitation photons, i.e., having a frequency that is twice that of the excitation frequency. Sum frequency generation is a nonlinear optical process wherein two photons of different excitation wavelength or frequency interact with a nonlinear material or molecule and are re-emitted or scattered as a single photon having an energy equal to the sum of that for the two excitation photons, i.e., having a frequency equal to the sum of the two excitation frequencies. Difference frequency generation is a nonlinear optical process wherein two photons of different excitation wavelength or frequency interact with a nonlinear material or molecule and are re-emitted or scattered as a single photon having an energy equal to the difference of that for the two excitation photons, i.e., having a frequency equal to the difference of the two excitation frequencies. Throughout this disclosure, the terms SHG, SFG, and DFG may be used interchangeably, as will be understood by those of skill in the art. Two-photon fluorescence is a nonlinear optical process wherein two photons of the same excitation wavelength or frequency interact with a nonlinear material or molecule and are absorbed by the material or molecule, followed by emission as a single photon having higher energy, i.e., having a higher frequency and a shorter wavelength, than the excitation photons.

Nonlinear-active: As used herein, the phrase “nonlinear-active” refers to molecules, labels, or tags that are second harmonic-active (SH-active or SHG-active), sum frequency-active (SF-active or SFG-active), difference frequency-active (DF-active or DFG-active), or two-photon fluorescence-active (TPF-active), i.e., that are capable of generating second harmonic light, sum frequency light, difference frequency light, or two-photon fluorescence respectively upon exposure to light of the appropriate wavelengths, intensities, and phases. Various methods employing TPF measurements and, optionally, SFG, SFG, or DFG measurements in conjunction are disclosed. In some cases, a molecule, label, or tag may be nonlinear-active such that it emits both second harmonic light, for example, and two-photon fluorescence upon exposure to light of the appropriate wavelengths, intensities, and phases.

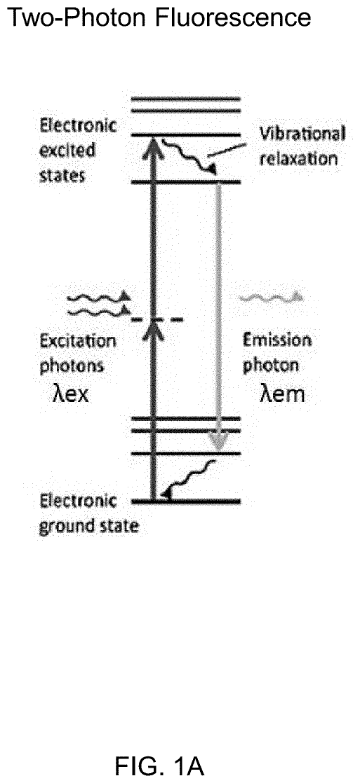

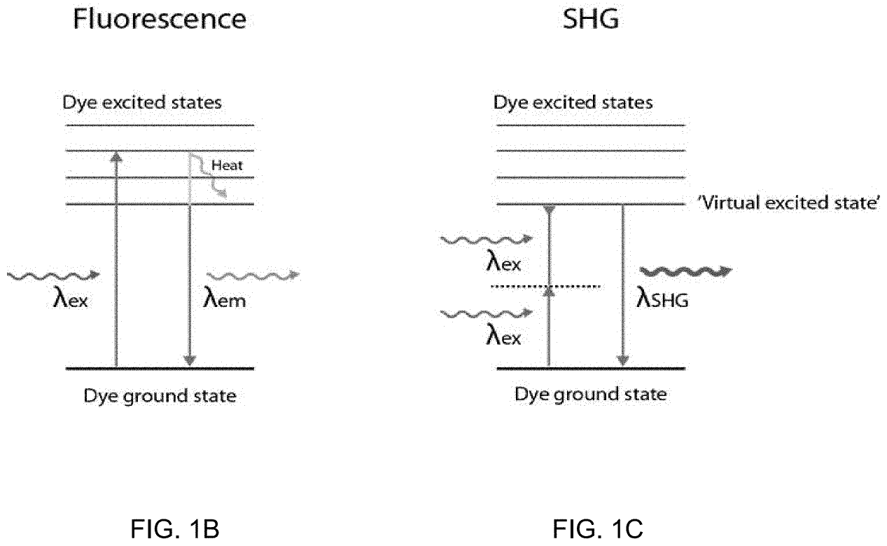

Detection of molecular orientation, conformation, and structure using two-photon fluorescence: Two-photon fluorescence (FIG. 1A), in contrast to the more widely used one-photon fluorescence-based techniques (FIG. 1B), is a nonlinear optical process in which two photons of the same excitation wavelength or frequency interact with a nonlinear material or molecule and are absorbed by the material or molecule, followed by emission as a single photon having higher energy, i.e., higher frequency and shorter wavelength, than the excitation photons (FIG. 1A). As used herein, the term “nonlinear optical process” may refer to two-photon fluorescence, second harmonic generation, sum frequency generation, or difference frequency generation. In general, a nonlinear optical process is excited by illuminating a nonlinear-active label with excitation light of at least one fundamental frequency.

Two-photon fluorescence depends on the angle ϕ of the two-photon transition dipole moment (TDM) relative to the normal to the surface plane to which the two-photon-active probes (molecules) are attached and on the angle between the polarization of the excitation light and the surface plane normal, θ. The equation governing the intensity of measured TPF can thus be written in the limit of low-NA pinhole detection as:

I_{TPF}(θ, ϕ) ∝ I₀ (f⁴∂_{TPF} cos⁴ θ+3f² [symbol] sin⁴ ϕ cos² ϕ [symbol] cos² θ sin² θ+3/8∂′_{TPF} sin⁴ θ) (1)

where Iₒ is the maximum intensity, f is a constant that accounts for losses in power on the prism surfaces used to couple the excitation light to the substrate surface (a known value), and where the following TPF order parameters are defined as:

∂_{TPF}= [symbol] cos⁴ ϕ sin² ϕ

[symbol]

∂′_{TPF}= [symbol] sin⁶ ϕ

[symbol]

and are trigonometric moments of a probability density function with integration occurring over the orientational distribution of the probe (TPF-active) molecules in the sample under study.

By making TPF measurements using two different polarizations of the excitation light and taking the ratio of the measured intensities, equation (1) can be rearranged to yield the following relationship between the measured intensity ratios and mean tilt angle, ϕ:

$$\frac{〈 {cos}^{4} {φsin}^{2} φ 〉}{〈 {sin}^{6} φ 〉} = \frac{3}{8} \frac{1}{{f}^{4}} \frac{{I}_{p}}{{I}_{s}} . \qquad ( 2 )$$

where Iₚ is the TPF signal measured using P-polarization, Iₛ is the TPF signal measured using S-polarization and the brackets (< >) denote an average value.

Unlike the case with SHG and other surface-selective techniques, TPF background signal can simply be subtracted linearly from the total TPF signal—either in the presence or absence of ligand—to determine the TPF signal arising from probes attached to the biomolecule of interest without the need to determine the phase between them, as is required for parsing the SHG probe-only signal from background signal. This provides a valuable solution to the vexing problem of determining whether a ligand is a true ligand or a false positive in screening assays where the ligand has a significant effect on the background signal in the absence of protein. With SHG, the protein-only signal must be de-convoluted from the total signal and the background signal, and this requires knowledge of the relative phases between the different signals which is often unknown or uncertain. With TPF, the difference between the total signal and the background signal directly yields the protein-only signal. Therefore, by using TPF to monitor structure or conformational change, the net change produced by the ligand on the protein-only signal can be determined to ascertain whether the ligand is a true positive. Compounds or ligands that are true positives and induce conformational change upon specific binding to protein will exhibit a net change in the TPF signal relative to any change they produce on the background surface alone.

Because TPF and SHG have different order dependences on the orientational distribution, they provide two independent equations that can be used either in one experiment or in separate experiments, in which the detection modality (e.g., TPF and/or SHG), protein sample, labeling site, etc., is varied, to obtain angular measurements of the underlying molecular orientational distribution.

Here, the use of TPF is disclosed in specific embodiments to obtain, for example, additional orientational information about labeled biological molecules. TPF measurements may be used alone or in combination with SHG, SFG, or DFG measurements to obtain, for example, values for the mean tilt angle of the TPF and/or SHG, SFG, or DFG label relative to the normal to the surface on which the labeled biological molecules are tethered. TPF has enhanced angular sensitivity to the orientational distribution of the labeled biological molecules as compared to one-photon fluorescence due to its higher order dependence on the tilt angle of the label (or probe). Moreover, in the case of a pinhole or low-NA detection scheme, with the detector placed directly above or below the sample of interest and along the axis normal to the surface, in the absence of a collecting lens, the TPF sensitivity is enhanced by a factor sin² ϕ. This additional sensitivity stems from the emission pattern of a probe molecule, which should radiate in a dipole pattern. Detection of a TPF signal relies upon two separate processes: two photons must be adsorbed by the probe molecule and a single photon must be emitted. In the absorption process, photon adsorption efficiency is dependent upon the tilt angle ϕ. Likewise, in the emission process, the fraction of photons emitted toward the detector is also dependent upon the tilt angle, ϕ. In the limit of low-NA with the detector positioned orthogonal to the surface directly above the probe molecules, the fraction of photons detected by the detector varies with sin² ϕ. Accordingly, the preferred embodiments of the present disclosure involving TPF generation and detection of structure or conformational change use the low-NA pinhole detection approach.

If the orientational distribution of the tethered probe molecules is fairly broad (e.g., >35 degrees), TPF tends to be much less sensitive to angular change than SHG. For example, described below are the results of calculations for assumed values of mean tilt angle and width (standard deviation) for a Gaussian distribution of TPF- and SHG-active probes tethered to a surface which disclose the sensitivity of the TPF measurements to mean tilt angle, with and without the sin² ϕ enhancement afforded by low-NA detection in the absence of an objective or collecting lens.

Use of a low-NA, pinhole detection format for TPF measurements: Unlike the technique known as total internal reflection fluorescence microscopy (TIRFM), the methods disclosed herein do not require a high-NA objective to produce the TIR condition. Accordingly, a prism may be used to implement the TIR excitation, and TPF is detected either above or below the sample plane. In a preferred embodiment, this prism/TIR excitation optical arrangement is used in combination with a planar sample—e.g., a supported lipid bilayer membrane to which biomolecules of interest labeled with TPF-active probes are attached—and a low-NA pinhole detector centered either directly above or below the focal spot in the sample plane. In this preferred embodiment, the sample of interest is tethered or immobilized on a glass surface (i.e., an optical interface) which itself is optically coupled to the prism below using for example, immersion oil or an optically coupling adhesive, as are well known to those skilled in the art. Importantly, fluorescence emitted by molecules in the sample plane does not pass through a lens but instead is detected by the detector as it passes either from the sample plane upwards, potentially through the volume of a sample if it is liquid-based or otherwise air, or from the sample plane downwards, through the prism. In a preferred embodiment, the sample of interest comprises tethered or immobilized molecules that are distributed in an isotropic fashion in the sample plane (i.e., azimuthally), or are assumed to be in the analysis of their orientational distribution. Furthermore, to ensure a high degree of polarization purity, another preferred embodiment comprises focusing the excitation light to a very narrow cone angle so that the light is essentially collimated at the total internal reflection angle and there is virtually no off-axis polarization. For example, in one embodiment, a 4 mm diameter laser beam is focused to a 50 μm diameter spot over a distance of 160 mm, thereby resulting in a full cone angle of about 1.5 degrees, or ˜0.8 degrees above and below the critical angle.

Two photon fluorescence sensitivity analysis under different detection schemes: To illustrate the sensitivity of TPF to mean angle and distribution width, calculations were performed to determine the magnitude of the TPF intensity change at a given mean angle and distribution width (assuming a Gaussian distribution) in changing to a mean angle 5° away with the same distribution width. The calculations below estimate the expected change in signal when the electric field of the laser is perpendicular to the surface normal (s-polarization). For a detector parallel to the surface normal positioned directly above or below the sample the number of detected photons from TPF should scale as <sin⁴ ϕ> in the case of high NA and <sin⁶ ϕ> in the case of low NA.

Integration of equation (2) (for TPF) or equation (6) (for SHG) over a Gaussian distribution can be performed using the following equation to determine pairs of tilt angle and distribution width that satisfy the equations. The brackets, <f(ϕ)>, in equations (2) and (6) indicate a normalized integral over the tilt angle ϕ from 0 to π of the expression f(ϕ) multiplied by a Gaussian:

$$〈 f ( φ ) 〉 = \frac{{\int }_{0}^{π} exp [ - {( φ - {φ}_{0} )}^{2} \text{/} 2 {σ}^{2} ] f ( φ ) sin ( φ ) d φ}{{\int }_{0}^{π} exp [ - {( φ - {φ}_{0} )}^{2} \text{/} 2 {σ}^{2} ] sin ( φ ) d φ} \qquad ( 3 )$$

where ϕ₀ is the mean angle and σ is the distribution width. Since a Gaussian function has an infinite extent and the integral is evaluated between 0 to π, the Gaussian was “folded” into the integral by summing all contributions between −4π and 4π following the procedure outlined by Simpson and Rowlen (Simpson and Rowlen (1999), J. Am. Chem. Soc. 121:2635-2636). This will produce integrals that are valid for distribution widths up to 70°. The results of the calculations are summarized in Table 1.

TABLE 1
Comparison of TPF signals collected using high NA and low NA optical schemes

| Initial State | | Final State | | | |
|---|---|---|---|---|---|
| | Distribution | | Distribution | Without low- | With low-NA |
| Mean Angle (°) | Width (°) | Mean Angle (°) | Width (°) | NA (%) | (%) |
| 30 | 40 | 35 | 40 | 6 | 8 |
| 30 | 20 | 35 | 20 | 20 | 28 |
| 20 | 40 | 25 | 40 | 6 | 8 |
| 20 | 20 | 25 | 20 | 26 | 34 |
| 50 | 20 | 55 | 20 | 14 | 18 |
| 60 | 20 | 65 | 20 | 10 | 13 |
| 15 | 20 | 20 | 20 | 29 | 38 |
| 15 | 20 | 20 | 15 | 41 | 56 |
| 30 | 15 | 35 | 15 | 30 | 41 |

One can see from the results summarized in Table 1 that, for values of mean angle and distribution widths across the range of possible values, the low-NA detection approach (i.e., without the use of a lens) offers significant increases in sensitivity to angular change.

Moreover, from these calculations one also discovers that, if the orientational distribution of the two-photon active probes is narrow (≤25°), the sensitivity of the TPF measurements to mean tilt angle approaches that of SHG. For example, at a mean angle of 30° and a width of 15°, the SHG signal changes by 10% going to an angle of 32° with the width remaining the same at 15°; for the same initial and final states, TPF with low-NA detection without a lens changes by 15%. Similarly, for a mean angle of 20° and a width of 25°, SHG changes by 18% going to a mean angle of 25° and a width of 25°; TPF with low-NA detection changes by 24%. Accordingly, a key aspect of the present invention, and one preferred embodiment, involves the use of at least one sample (e.g., a protein sample) containing an exogenously attached probe, dye, or unnatural amino acid, or a genetically incorporated unnatural amino acid or other label, wherein the measured probe orientational distribution has a width of less than or equal to 25° (degrees).

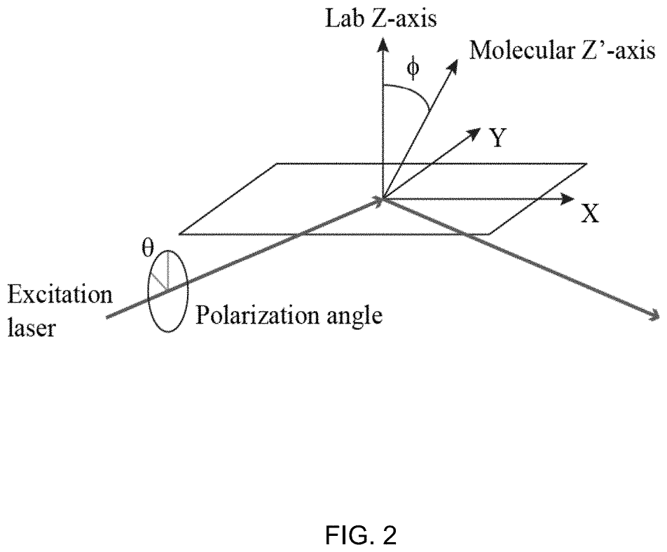

Measurement of TPF transition dipole moment (TDM), and optionally SHG χ⁽²⁾, and the relationship to protein structure: In general, the methods, devices, and systems disclosed may rely on the use of two-photon induced fluorescence (TPF) and, optionally, second harmonic generation (SHG) or the related nonlinear optical techniques of sum frequency generation (SHG) or difference frequency generation (DFG), for the determination of molecular orientation, conformation, structure, or changes thereof. In these methods, polarization-dependent measurements are used to determine the components of the transition dipole moment (TDM) of the two-photon absorption transition (or the components of the hyperpolarizability, χ⁽²⁾, for SHG and the related nonlinear optical techniques as discussed below). The values of TPF intensity or the components of χ⁽²⁾ for SHG may be measured in the laboratory frame of reference using polarized excitation light of at least one fundamental frequency. Some light sources, e.g., some lasers, produce light of a fundamental frequency that is substantially polarized. In some embodiments, the polarization of the excitation light may be further defined and/or adjusted using one or more optical polarizers, wave plates, etc. Typically, the plane of incidence of the polarized light (i.e. the plane defined by the propagation direction of the excitation light and a vector perpendicular to the plane of the substrate or reflecting surface) will be the X-Z plane of the laboratory coordinate system illustrated in FIG. 2. Polarized light having its electric field vector parallel to the plane of incidence is called p-polarized light. Polarized light having its electric field vector perpendicular to the plane of incidence is called s-polarized light. In some embodiments, the polarization of the detected second harmonic light generated by excitation of a nonlinear-active moiety may also be defined and/or adjusted using one or more optical polarizers, wave plates, etc. As outlined above, by measuring two-photon fluorescence intensities using at least two different polarizations of the excitation light, one can use the resulting information on relative orientation of the labels to develop a model for protein structure and detect changes thereof using two-photon fluorescence alone or two-photon fluorescence in conjunction with SHG measurements or related nonlinear optical measurement techniques.

In some embodiments, TPF is used in combination with SHG measurements and detected at one or more polarizations of the excitation light in order to obtain additional orientational information about a probe attached to a biomolecule, which in turn is tethered to a surface, for the purpose of obtaining structural information about the biomolecule. For example, because TPF depends on a different order parameter of probe orientation than SHG, it offers an independent equation that allows one to solve simultaneously for two separate orientational parameters, for example the mean and the width of a Gaussian distribution of probe molecules. In preferred embodiments, detection of the TPF signal is accomplished using a low-NA or pinhole detection apparatus as described below and results in an even higher order dependence on probe orientation, and thus enhances sensitivity. In some embodiments, the highest degree of confidence in angular measurements determined by SHG and TPF occurs when the probe and the biomolecule are tethered to a surface in a relatively narrow orientational distribution, wherein for at least one probe location within the biomolecule the angular distribution of the probe as determined by combined SHG and TPF measurements and assuming a Gaussian distribution, results in an orientational distribution width of 35 degrees or less (≤35°). In some embodiments, the highest degree of confidence in angular measurements determined by SHG and TPF occurs when tethering of the labeled biomolecule results in a relatively narrow orientational distribution width of less than or equal to 30°, less than or equal to 25°, or less than or equal to 20°. In some embodiments, the labels or probes are TPF-active and, optionally, also SHG-, SFG-, or DFG-active, and can be incorporated at specific sites within a biomolecule of interest such as a protein using techniques known to those skilled in the art such as, for example, incorporation of nonlinear-active unnatural amino acids. In some embodiments, such probes are incorporated at single sites within a single biomolecule construct, whereas in other embodiments two or more probes are incorporated at multiple sites within a single biomolecule construct.

In some embodiments, TPF is measured from biomolecules labeled with TPF-active probes in order to ascertain whether the labeled biomolecules are attached to the surface, which is not always evident using an SHG measurement alone since, if the net average orientation of the SHG probes is relatively flat relative to the surface normal, the signal will be relatively small, whereas the same sample will generally produce a correspondingly high TPF signal. Thus the amount of signal produced by an ensemble of probes that are both TPF-active and SHG-active tends to be anti-correlated in the two techniques as described in more detail below in the theoretical background.

In some embodiments, a biomolecule may be labeled with a probe that is only SHG- or only TPF-active and the measurements of each experiment may be compared to the other. In some cases, a biomolecule may be labeled at one site, whereas in other embodiments many different versions of a biomolecule are created, each bearing a probe at a unique, single site that is both SHG- and TPF-active. In other embodiments, different versions of a biomolecule are created each bearing a probe at a unique, single site that is either i) TPF-active or ii) SHG-active (or SFG- or DFG-active).

Obtaining structural information from X-ray crystallography or NMR methods, which are of limited value for drug discovery applications due to factors such as throughput, sensitivity, the use of non-physiological conditions, the size of protein amenable to the technique, and so on, can be challenging. Thus, another aspect of the present invention is to provide a site-specific readout of conformation at functionally relevant sites in a protein or other biomolecule. Protein sites that are “functionally relevant”, as defined herein, include any sites which make direct or indirect structural contact with a binding partner (e.g., an effector molecule) as determined by a structural technique such as X-ray crystallography, NMR, or SHG. Direct structural contact is defined as any amino acid or other structural residue, some portion of which is within 2 nm of some portion of the binding partner molecule. Indirect structural contact is defined as any amino acid or other structural residue, some part of which changes its orientation, conformation or relative coordinates upon binding of binding partner (e.g., an effector molecule), or a binding partner mimic or analog, as seen by a structural technique such as X-ray, NMR or SHG, relative to its orientation, conformation, or relative coordinates in the absence of the binding partner, mimic or analog. The term “functionally relevant” also includes residues which are known to be important in the binding or the modulation (e.g., activation, inhibition, regulation, and so on) of the binding molecule by non-structural means (e.g., mutagenesis or biochemical data which shows that particular residues are important for binding or modulation of the binding partner).

In some embodiments, the TPF structural data, and optionally, the SHG structural data, obtained using the disclosed methods may be overlaid or combined with structural data from protein crystallographic studies, NMR studies, UV-Vis and fluorescence spectroscopic studies, circular dichroism studies, cross-linking experiments, small-angle X-ray scattering studies, etc. In some embodiments, the methods further comprising globally fitting data for the relative orientation of the one or more nonlinear-active labels to a structural model of the protein molecule, wherein the structural model is based on known positions of the one or more nonlinear-active labels within the protein molecule. Optionally additional structural measurements or constraints can be employed in determining such a model, e.g. data from X-ray, NMR measurements, or other experimental measurements.

In some embodiments, TPF and/or SHG signal measurements may be performed under a variety of experimental conditions, as discussed below, where different experimental conditions result in a change of the orientational distribution of the labeled molecules tethered to the optical interface. Each set of experimental conditions that leads to a different set of measured values for the TPF transition dipole moment, and optionally SHG χ⁽²⁾, due to a different underlying orientational distribution in the lab frame, allows for independent measurements of the tilt angle ϕ to be determined by TPF and optionally SHG. By combining two or more such measurements, a more accurate determination of protein structure(s) can be made, including structure(s) of protein that exist in an equilibrium of multiple conformational states. Measurements of the components of the TPF transition dipole moment, and optionally SHG χ⁽²⁾, and determination of the values for ϕ can be used to develop structural models through the use of standard molecular modeling techniques known to those of skill in the art and, in some embodiments, a choice of appropriate simplifying assumptions. One non-limiting example of an assumption that may be made to simplify the analysis and develop protein structural models is that, although the orientation of the TPF-active and/or SHG-active label on the protein surface varies from one experimental condition to another in the laboratory frame of reference (i.e., relative to the axis normal to the surface plane), the orientation of the TPG-active and/or SHG-active label relative to the protein frame of reference remains constant under different experimental conditions. In effect, under this assumption one varies the orientational distribution of the proteins on the surface in ways that do not perturb their function and conformational landscape. Each experimental condition produces at least one independent equation relating the measured TPF TDM and optionally SHG intensity at the different polarizations to the molecular orientational distribution. Appropriate controls such as ligand-induced conformational changes, ligand competition experiments, kinetics of ligand binding, dose-response measurements, and others, can be run at each experimental condition to ensure that the protein is still functional and thus native-like. The measurements of mean angle, for example, along with other parameters of the orientational distribution of the label or two-photon-active or hyperpolarizable moiety in the protein, can be used as constraints in de novo or integrative structural model building according to methods known to those skilled in the art. In some embodiments, for example, an apo X-ray crystallographic structure of a protein may be included in the model, and overlaid with structural data provided by TPF and/or SHG measurements to improve the accuracy of the model.

Non-limiting examples of assumptions that may be made in some embodiments of the disclosed method for the purpose of simplifying the analysis of the SHG structural data include: (i) that a single component of the TPF TDM and optionally of the α⁽²⁾ term (e.g., α_{zzz}⁽²⁾) dominates the two-photon absorption tensor (and optionally for SHG, the hyperpolarizability of the label); (ii) that the position of the label(s) within the protein (i.e., the identities of the amino acid residues to which they are attached) is known; and (iii) that the orientation of the tethered or immobilized protein molecules is isotropic in the X-Y plane (i.e., they are randomly oriented on the plane of the substrate surface or in the plane of a supported lipid bilayer).

In one example of the disclosed methods, a protein is labeled at a single site-specifically engineered cysteine residue with a two-photon active and optionally SHG-active label, which in turn possesses a single dominant element of the two-photon absorption tensor and optionally the α⁽²⁾=α⁽²⁾_{z′z′z′}. The labeled protein is attached via a His tag to a supported lipid bilayer membrane which comprises Ni-NTA moieties attached to lipid head groups. A baseline TPF signal and optionally an SHG signal is generated in this way, and the non-vanishing components of χ⁽²⁾ are given, as is well known to those of skill in the art (Salafsky, J. S. (2001), “‘SHG-labels’ for Detection of Molecules by Second Harmonic Generation”, Chemical Physics Letters 342, 485-491; Salafsky, J. S. (2003), “Second-Harmonic Generation as a Probe of Conformational Change in Molecules”, Chemical Physics Letters 381, 705-709; Salafsky, J. S. (2006), “Detection of Protein Conformational Change by Optical Second-Harmonic Generation”, Journal of Chemical Physics 125), by the equations:

χ_{zzz}⁽²⁾=Nₛ [symbol] cos³ ϕ [symbol] α_{z′z′z′}⁽²⁾

χ_{zxx}⁽²⁾=χ_{xzx}⁽²⁾=1/2Nₛ [symbol] sin² ϕ cos ϕ [symbol] α_{z′z′z′}⁽²⁾. (4)

where Nₛ and α_{Z′Z′Z′}⁽²⁾ are the surface density and molecular hyperpolarizability, respectively. The components of χ⁽²⁾ can then be determined from two different polarization-dependent measurements (I_{zzz} and I_{zxx}, or equivalently Iₚₚₚ and Iₚₛₛ). In this case, χ_{zzz}⁽²⁾ can be determined by measuring the p-polarized SHG signal using p-polarized fundamental excitation light. For example, if fundamental excitation light at 800 nm is used (e.g., from a Ti: Sapphire mode-locked laser), the second harmonic signal is detected at 400 nm. In general, Iₚₚₚ, which is the SHG signal intensity observed under p-polarized excitation and p-polarized SHG detection, is governed by several components of the nonlinear susceptibility. However, a simplified approach for isolating only χ_{zzz}⁽²⁾ in this measurement is achieved by measuring the SHG signal at the critical angle of incidence in a total reflection geometry using a silica prism. In total internal reflection (TIR) geometry the measured SHG intensity is determined by the refractive indices of the prism and the buffer in which the surface-tethered proteins are bathed, since the off-axis tensor components of χ⁽²⁾ vanish, leaving only χ_{zzz}⁽²⁾ which determines the measured Iₚₚₚ SHG signal intensity (referring to polarization of the fundamental/SHG beams). Similarly, χ_{zxx}⁽²⁾ can be determined by measuring Iₚₛₛ using s-polarized fundamental light and measuring the p-polarized SHG light intensity. In cases where Nₛ and α_{z′z′z′} are unknown, ratios of the intensities measured under different polarization combinations can be used to eliminate these parameters, leaving only ratios of the orientational distributions themselves which are trigonometric functions of ϕ, where ϕ is defined as the mean angle between the z-axis in the molecular frame and the surface normal. When the orientational distribution is narrow, ϕ can be determined directly. By repeating the measurements of the SHG intensity under different polarizations (e.g., I_{zzz} and I_{zxx}) using protein labeled at two or more different sites (e.g., in two or more different single-site cysteine mutants, at these cysteine sites), one can obtain, for example, a different ϕ for each label site that can be used as a constraint in structure determination. Each measurement requires a labeled protein, preferably with the label site-specifically attached (e.g., covalently attached via site-directed cysteine mutagenesis) at a known position within the protein. By also varying the experimental conditions to produce differently oriented protein relative to the surface plane along with the two or more different sites of labeling, independent measurements can be made to determine multiple parameters describing the orientational distribution, thereby providing important constraints for protein structure determination. A key step of the present invention is to make measurements of a protein labeled at two or more different sites (preferably in separate protein-label conjugates) under two or more different experimental conditions that result in different values of χ⁽²⁾, which depends on the underlying orientational distribution,or ratios of χ⁽²⁾ components (e.g., χ_{zzz}⁽²⁾/χ_{zxx}⁽²⁾). By measuring values of χ⁽²⁾ for the same protein labeled at two or more different sites under two or more different experimental conditions, one can obtain more accurate measurements of the ϕ's (in the lab frame) and relate the difference between them, i.e., in the protein frame, to the structure of the protein.

Multiple conformations-equilibrium orientational distributions: If a protein (or other biomolecule) exists in an equilibrium of multiple conformational states, the protein may be described by a multi-modal (or multi-state) orientational distribution at each label site. If the distribution is composed of a sum of Gaussians with different weights, mean angles (ϕ's) and distribution widths (σ's), a complete description of the protein's conformational landscape will depend on determining each of these parameters. For example, if the local structure of the protein at label site 1 adopts 3 conformations, under these assumptions, the local orientational distribution may be described by 3×3 parameters to be determined, or 9 unknowns, describing amplitude, mean angle, and width for each conformation. Label site 2 may adopt only 2 local conformations and in that case can similarly be described by 6 parameters. The additional independent measurements, an aspect of the present invention, can be obtained by varying experimental conditions such as the tag site (e.g., C- and N-terminus), fusion protein sequence, tag length (e.g., 6×, 8×, 10×, and 12× His tags), buffer conditions (e.g., different salt concentrations), and so on, or in general, any experimental condition that varies the orientation of the protein on the surface and thus impacts the measured values of χ⁽²⁾. All of these independent measurements can then be used, for example, in a global fitting method to determine the solution-based conformational landscape (i.e. multi-state orientational distribution) in the protein frame of reference at these two sites. In some embodiments, the X-ray crystal structure coordinates of the protein may optionally be used as a further constraint in the model building.

Detection of molecular orientation, conformation, and structure using SHG and related nonlinear optical techniques: SHG and the related technique sum-frequency generation (SFG) have been used in the past to study the orientation of dye molecules at an interface (Heinz T., et al., (1983), “Determination of Molecular Orientation of Monolayer Adsorbates by Optical Second-Harmonic Generation”, Physical Review A 28(3):1883-1885; Heinz, T, (1991) Second-Order Nonlinear Optical Effects at Surfaces and Interfaces”, in Nonlinear Surface Electromagnetic Phenomena (Stegeman, H. P. a. G. ed.), Elsevier, Amsterdam, pp 353-416). In these measurements, the components of the nonlinear susceptibility (χ²) of the labeled interface are determined using polarized light. Details of the molecular orientation distribution for the dye molecules at the interface can then be inferred using the experimentally determined values for χ² and assumptions regarding the degree of orientation of the dye molecules within the plane of the interface, the relative magnitude of the components of hyperpolarizability (α²) of the dye molecules in the molecular frame of reference, etc.

The use of SHG and the related nonlinear optical techniques of SFG and DFG for detection of biomolecule binding events at a surface, for measurement of tilt angles, or for measurement of conformational changes in proteins has been disclosed in previous technical publications and patent applications (see, for example, Salafsky, J. (2006), “Detection of Protein Conformational Change by Optical Second-Harmonic Generation”, J. Chem. Phys. 125:074701; U.S. Pat. Nos. 6,953,694; and 8,497,073). In general, these disclosures describe the use of SHG or other nonlinear optical techniques to measure changes in signal upon contacting a target protein with a binding partner, e.g., a ligand.

Second harmonic generation (FIG. 1C), in contrast to the more widely used one-photon fluorescence-based techniques (FIG. 1B), is a nonlinear optical process in which two photons of the same excitation wavelength or frequency interact with a nonlinear material and are re-emitted as a single photon having twice the energy, i.e., twice the frequency and half the wavelength, of the excitation photons. Second harmonic generation only occurs in nonlinear materials lacking inversion symmetry (i.e., in non-centrosymmetric materials), and requires a high intensity excitation light source. It is a special case of sum frequency generation and is related to other nonlinear optical phenomena such as difference frequency generation.

Second harmonic generation and other nonlinear optical techniques can be configured as surface-selective detection techniques because of their very high order dependence on the orientation of the nonlinear-active species. Tethering of the nonlinear-active species to a surface, for example, can create a net, average degree of orientation that is absent when molecules are able to undergo free diffusion in solution. An equation commonly used to model the orientation-dependence of nonlinear-active species at an interface is:

χ⁽²⁾=Nₛ [symbol] α⁽²⁾ [symbol] (5)

where χ₂ is the nonlinear susceptibility, α⁽²⁾ is the nonlinear susceptibility, Nₛ is the total number of nonlinear-active molecules per unit area at the interface, and <α⁽²⁾> is the average over all orientations of the nonlinear hyperpolarizability (α⁽²⁾) of these molecules. Typical equations describing the nonlinear interaction for second harmonic generation are:

α²(2ω)=β E(ω)E(ω) or

P²(2ω)=χ² E(ω)E(ω)

where α and P are, respectively, the induced molecular and macroscopic dipoles oscillating at frequency 2ω, β and χ² are, respectively, the hyperpolarizability and second-harmonic (nonlinear) susceptibility tensors, and E(ω) is the electric field component of the incident radiation oscillating at frequency ω. The macroscopic nonlinear susceptibility χ² is related to the microscopic β hyperpolarizability by an orientational average of α². The next order term in the expansion of the induced macroscopic dipole describes other nonlinear phenomenon, such as third harmonic generation. The third order term is responsible for such nonlinear phenomena as two-photon fluorescence. For sum or difference frequency generation, the driving electric fields (fundamentals) oscillate at different frequencies (i.e., ω₁ and ω₂) and the nonlinear radiation oscillates at the sum or difference frequency (ω₁±ω₂).

The intensity of SHG is proportional to the square of the nonlinear susceptibility, and is thus dependent on both the number of oriented nonlinear-active species at the interface and their orientational distribution. This property can be exploited to detect a conformational change. For example, conformational change in receptors can be detected using a nonlinear-active label or moiety wherein the label is attached to or associated with a receptor tethered to a surface; a conformational change leads to a change in the direction (orientation) of the label with respect to the surface plane and thus to a change in a physical property (e.g., intensity) of the nonlinear optical signal. The techniques are intrinsically sensitive to changes in the orientational distribution of labeled molecules at an interface, whether spatial or temporal.

By taking SHG measurements using two different polarizations of the excitation light and taking the ratio of the measured intensities, one can derive the mean tilt angle from the relationship between the measured intensity ratios and mean tilt angle, ϕ:

$$\frac{〈 {cos}^{3} φ 〉}{〈 cos φ 〉} = \frac{1}{1 + 2 {f}^{2} \sqrt{\frac{{I}_{pss}}{{I}_{ppp}}}} \qquad ( 6 )$$

where Iₚₚₚ is the SHG signal measured using P-polarization, Iₚₛₛ is the SHG signal measured using S-polarization, f is a constant that accounts for losses in power on prism surfaces used to couple the excitation light to the substrate surface (a known value), and the brackets (< >) denote an average value.

Accordingly, in some embodiments of the disclosed methods, the strong dependence of the SHG signal intensities measured using polarized excitation light on mean tilt angle, ϕ, (as indicated in equation (6)) is exploited for more sensitive detection of conformational changes in labeled proteins or other biomolecules by making measurements of ratios of the SHG signal measured using p-polarized and s-polarized excitation light under various detection polarizations, p-polarized SHG detection being the preferred embodiment with both pure p-polarized and pure s-polarized excitation, (e.g., χ_{zzz}⁽²⁾/χₓₓₓ⁽²⁾). In another embodiment a single polarization, which is a mixed state of p- and s-polarized light, can be used to generate SHG that itself is polarized in two orthogonal directions. By measuring the relative intensities of these two orthogonally polarized SHG signals by, for example, splitting the signal using a polarized beam splitting cube, an equation similar in form to equation (6) can be formulated to relay information about the mean tilt angle ϕ.

Second harmonic generation and other nonlinear optical techniques (including TPF, as discussed above) may be rendered additionally surface selective through the use of total internal reflection as the mode for delivery of the excitation light to the optical interface (or surface) on which nonlinear-active species have been tethered or immobilized. Total internal reflection of the incident excitation light creates an “evanescent wave” at the interface, which may be used to selectively excite only nonlinear-active labels that are in close proximity to the surface, i.e., within the spatial decay distance of the evanescent wave, which is typically on the order of tens of nanometers. In the present disclosure, the evanescent wave generated by means of total internal reflection of the excitation light is preferentially used to excite a nonlinear-active label or molecule. The efficiency of exciting nonlinear active species in the nonlinear-active processes described herein depends strongly on their average orientation relative to the surface. For example, if no net average orientation of the nonlinear active species exists, there will be no SHG signal.

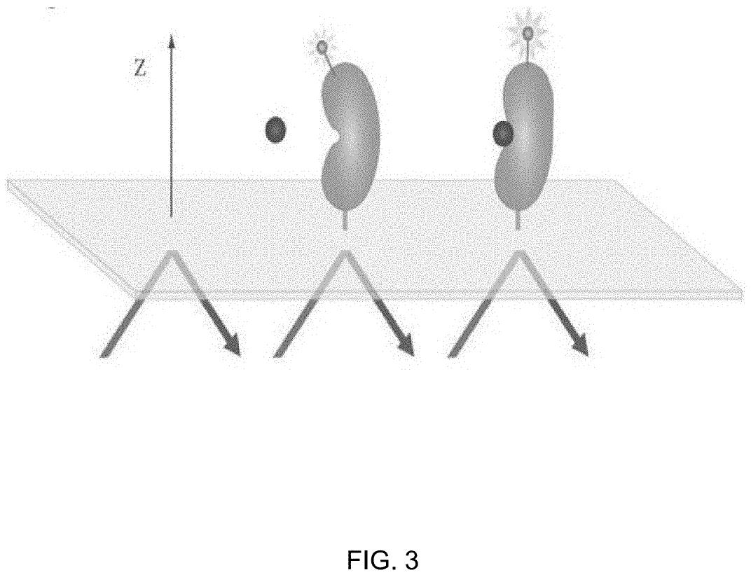

This surface selective property of SHG and other nonlinear optical techniques can be exploited to determine average orientation, conformation, structure, or changes thereof in biological molecules immobilized at interfaces. For example, conformational change in a receptor molecule due to binding of a ligand may be detected using a nonlinear-active label or moiety where the label is attached to or associated with the receptor in such a way that the conformational change leads to a change in the orientation or distance of the label with respect to the interface (FIG. 3), and thus to a change in a physical property of the nonlinear optical signal. In the past, the use of surface-selective nonlinear optical techniques has been confined mainly to applications in physics and chemistry, since relatively few biological samples are intrinsically non-linearly active. Recently, the use of second harmonic active labels (“SHG labels”) and other nonlinear-active labels has been introduced, allowing virtually any molecule or particle to be rendered highly non-linear active. The first example of this was demonstrated by labeling the protein cytochrome c with an oxazole dye and detecting the protein conjugate at an air-water interface with second harmonic generation [Salafsky, J., “‘SHG-labels’ for Detection of Molecules by Second Harmonic Generation”, Chem. Phys. Lett. 342(5-6):485-491 (2001)]. Techniques for labeling or otherwise rendering target proteins, biological drug candidates, reference drugs, and other biological entities nonlinear-active will be described in more detail below.

Surface-selective SHG, SFG, and DFG nonlinear optical techniques are also coherent techniques, meaning that the fundamental and nonlinear optical light beams have wave fronts that propagate through space with well-defined spatial and phase relationships. The use of surface-selective nonlinear optical detection techniques for analysis of conformation of biological molecules or other biological entities has a number of inherent advantages over other optical approaches, including: i) sensitive and direct dependence of the nonlinear signal on the orientation and/or dipole moment(s) of the nonlinear-active species, thereby conferring sensitivity to conformational change; (ii) higher signal-to-noise (lower background) than fluorescence-based detection since the nonlinear optical signal is generated only at surfaces that create a non-centrosymmetric system, i.e., the technique inherently has a very narrow “depth-of-field”; (iii) as a result of the narrow “depth of field”, the technique is useful when measurements must be performed in the presence of a overlaying solution, e.g., where a binding process might be obviated or disturbed by a separation or rinse step. This aspect of the technique may be particularly useful for performing equilibrium binding measurements, which require the presence of bulk species, or kinetics measurements where the measurements are made over a defined period of time; (iv) the technique exhibits lower photo-bleaching and heating effects than those that occur in fluorescence, due to the facts that the two-photon cross-section is typically much lower than the one-photon absorption cross-section for a given molecule, and that SHG (and sum frequency generation or difference frequency generation) involves scattering, not absorption; (v) minimal collection optics are required and higher signal to noise is expected since the fundamental and nonlinear optical beams (e.g., second harmonic light) have well-defined incoming and outgoing directions with respect to the interface. This is particularly advantageous compared to one-photon fluorescence-based detection, as fluorescence emission is isotropic and there may also be a large fluorescence background component to detected signals arising from out-of-focal plane fluorescent species. The signals arising from SHG, SFG or DFG provide an instantaneous, real-time means of studying a molecule's structure, conformation or change thereof such as occurs, for example, upon ligand binding. This property may be very useful in the disclosed methods for obtaining real-time “movies” of proteins undergoing structural changes as part of their function in real time.

If background SHG signal is present, due for example to the substrate-buffer interface, this can be “subtracted” out in various ways. For example, the phase difference between the SHG signal from the label on the protein and the SHG signal due to the background can be measured in an interferometric experiment such as the one described in Reider, G., et al. (1999), “Coherence Artifacts in Second Harmonic Microscopy”, Applied Physics B-Lasers and Optics 68, 343-347, or in the experiments described by Clancy and Salafsky (2017), “Second-Harmonic Phase Determination by Real-Time In Situ Interferometry”, Phys. Chem. Chem. Phys. 19:3722-3728. The SHG signal due to the labeled protein alone can then be determined.

Examples of the physical properties of second harmonic light and related nonlinear optical signals that may be monitored for the purposes of structure determination, structural comparison, and/or detection of conformational change include, but are not limited to, intensity, polarization, wavelength, the time-dependence of the intensity, polarization, or wavelength, or any combination thereof.

Normalization of SHG signals: In any of the embodiments disclosed herein, the method for identifying the position of the ligand binding site or ligand binding region within a biomolecule of interest may further comprise simultaneous or serial measurement of a two-photon fluorescence (TPF) signal, and its use for calculating an SHG:TPF signal ratio (or an SFG:TPF signal ratio, DFG:TPF signal ratio, or p-polarized/s-polarized TPF or SHG ratio as described herein) for the purpose of normalizing the measured nonlinear signal to the number of molecules tethered per unit area of the interface (i.e., the surface density or number density of tethered molecules on the interface). For example, in some embodiments, the nonlinear-active (i.e., SHG-active, SFG-active, or DFG-active) label used to label a target protein prior to tethering it to an interface may also produce two-photon fluorescence when illuminated with light of a fundamental frequency that is the same as or different than that used to generate second harmonic, sum frequency, or difference frequency light. In some embodiments, the target protein may be labeled with a two-photon fluorescent label that is different than the SHG-active, SFG-active, or DFG-active label. Because the two-photon fluorescence signal is linearly related to the number of labeled molecules being excited, the two-photon fluorescence signal provides a means for normalizing the SHG (or SFG or DFG) signal to correct for variations in the surface density of the tethered molecules, and thus facilitates the comparison of signals measured for different samples of labeled protein. In some embodiments, the two-photon fluorescence may be excited by delivery of the fundamental light (i.e., the excitation light, which is typically provided by a laser) to the interface using total internal reflection. In some embodiments, the two-photon fluorescence may be excited by delivery of the fundamental light in a direction that is orthogonal to the plane of the interface (e.g., using an epifluorescence optical setup), or at an arbitrary angle that is not orthogonal to the plane of the interface. In some embodiments, the two-photon fluorescence that is excited upon illumination with the fundamental frequency light may be detected and measured using an epifluorescence optical setup, e.g., wherein the emitted two-photon fluorescence is collected using a microscope objective. In some embodiments, the two-photon fluorescence may be detected and measured using a low-NA pinhole (i.e., without the use of a lens) positioned either directly above or below the point at which the excitation light is focused and oriented such that it is parallel to the plane of the interface. Two-photon fluorescence light passing through a collection lens, a microscope objective, or a pinhole (with or without the use of any intermediate optical elements such as additional lenses, mirrors, dichroic reflectors, bandpass filters, and/or apertures) may then be detected using a photomultiplier or other suitable detector. An example of a TPF- and SHG-active probe that is specific for cysteine residues under appropriate reaction conditions is 1-(2-maleimidylethyl)-4-(5-(4-methoxyphenyl) oxazol-2-yl)pyridinium methanesulfonate.

Determination of structural similarity and comparison of protein structures from different samples or at different points in time: As noted above, in a first aspect of the present disclosure, the use of SHG or related nonlinear optical baseline signals (e.g., SFG and DFG baseline signals) for the comparison of protein structures (or other biomolecule structures) from different samples or at different points in time is described. A key difference between prior disclosures and the present invention is the recognition that the baseline SHG signal itself provides a valuable tool for comparison of protein structure in different protein samples or at different points in time. Because of its extreme sensitivity to the net orientation of the nonlinear-active label, measurement of a baseline SHG signal upon illumination of labeled protein molecules from different samples (e.g., that have been labeled using an identical labeling method and that have been tethered to or immobilized on an optical interface, e.g., the surface of a glass substrate, using an identical tethering or immobilization technique) of in the same sample at different points in time with light of a fundamental frequency provides a convenient and sensitive tool to detect subtle differences in the structure of the protein. Protein products that are nominally identical should have substantially identical baseline SHG signals. Protein products that differ slightly in their tertiary structure, e.g., as a result of slight differences in folding during production, or due to slight differences in stability in a given buffer formulation, should have different baseline SHG signals. Fully-denatured protein should have zero measurable baseline SHG signal (i.e., as a consequence of having no net orientation of the nonlinear-active label on the optical interface). Comparison of the baseline SHG signals for labeled protein samples drawn from different steps in a production process, or from different lots of protein produced by the same production process, or for a given protein sample at different points in time, or for putatively identical protein products produced by different production processes, should thus provide a useful tool for optimizing and monitoring production processes, monitoring protein stability upon exposure to different reagents or upon subjecting them to different experimental conditions, monitoring the output of a production process (e.g., for quality control), and for evaluating similar protein products on a structural basis (e.g., for demonstrating biosimilarity between a biological drug candidate and a reference drug, or for demonstrating the structural equivalence of monoclonal and/or polyclonal antibodies used in clinical diagnostic tests).

In some embodiments, these comparisons may rely solely on measurements of SHG baseline signal (or SFG or DFG baseline signals). Successful reduction to practice of the disclosed approach requires the identification and elimination of all potential sources of error in the baseline SHG signal other than differences in protein tertiary structure, e.g., differences in labeling specificity or yield, differences in binding site density on the optical interface, differences in tethering or immobilization efficiency, etc.

In some embodiments, these comparisons may be made using a ratio of the SHG baseline signal (or SFG or DFG baseline signal) to a TPF baseline signal measured for the same sample, where the SHG baseline signal and the TPF baseline signal are measured either simultaneously or serially. In a preferred embodiment, a single nonlinear-active label that is both second harmonic-active and two-photon fluorescence-active may be used to label the protein sample. Because the TPF signal is linearly proportional to the surface density of labeled proteins tethered to the surface (the SHG signal is proportional to the square of the number of labeled proteins tethered per unit area, as discussed above), the use of SHG-to-TPF baseline signal ratios allows one to normalize the SHG baseline signal and correct for variations in the surface density of labeled protein molecules.

Protein samples: The disclosed methods, devices, and systems may be utilized to monitor protein structural variation in samples of any of a variety of purified or non-purified proteins. Examples of proteins for which the approach is suitable include, but are not limited to, enzymes, receptors, antibodies, monoclonal antibodies, polyclonal antibodies, humanized antibodies, IgG antibodies, IgM antibodies, IgA antibodies, IgD antibodies, IgE antibodies, fusion proteins or other genetically-engineered proteins, and subunits or fragments thereof. In some embodiments, the protein may be a biological drug or candidate drug.

In one preferred embodiment, the protein for which tertiary structure is to be monitored may be a protein that has been genetically-engineered to incorporate a unique labeling site for attaching a nonlinear-active moiety (i.e., a nonlinear-active label or tag that is SHG- and/or TPF active), and/or one that has been genetically-engineered to incorporate an unnatural amino acid that is intrinsically nonlinear-active. Examples of unique label-attachment sites that may be genetically-incorporated include, but are not limited to, incorporation of a lysine, aspartate, glutamate, or cysteine residue at an amino acid sequence position that is known to be located on the surface of the protein when the protein is properly folded. In the case of lysine incorporation, a nonlinear-active label may then be conjugated to the primary amine of the lysine residue using any of a variety of conjugation chemistries known to those of skill in the art. Similarly, in the case of aspartate or glutamate incorporation, a nonlinear-active label may then be conjugated to the carboxyl group of the aspartate or glutamate residue. In the case of cysteine incorporation, a nonlinear-active label may then be conjugated to the sulfhydryl group of the cysteine residue. In the case of methionine labeling, the general approach described herein provides a good approach provided that a SHG- and TPF-active probe with the required chemical handle is available or synthesized. As noted, in some cases, labeling may comprise genetic incorporation of an intrinsically nonlinear-active unnatural amino acid. One non-limiting example of an unnatural amino acid that is intrinsically nonlinear-active is Aladan described in Cohen, et al. (2002), “Probing Protein Electrostatics with a Synthetic Fluorescent Amino Acid”, Science 296(5573):1700-1703. Other examples of suitable labeling techniques will be discussed in more detail below.

In another preferred embodiment, the protein for which tertiary structure is to be monitored may be a protein that has been genetically-engineered to incorporate a unique tethering or immobilization site for attaching the protein to the optical interface, and/or one which has been genetically-engineered to incorporate an unnatural amino acid residue that serves as a unique tethering or immobilization site for attachment of the protein to the optical interface or for attaching an SHG- or TPF-active probe in a biorthogonal fashion. Examples of unique tethering or immobilization sites that may be genetically-incorporated include, but are not limited to, incorporation of a lysine, aspartate, glutamate, methionine, or cysteine residue at an amino acid sequence position that is known to be located on the surface of the protein when the protein is properly folded. The protein may then be tethered to or immobilized on the optical interface using any of a variety of conjugation and linker chemistries known to those of skill in the art. Another non-limiting example of a unique tethering or immobilization site that may be genetically-incorporated into a protein product may be a His tag (e.g., a series of 1, 2, 3, 4, 5, 6, 7, 8, 9, 10, 11, 12, or more than 12 histidine residues) that may then provide an attachment site for binding to Ni/NTA groups attached to the optical interface. One non-limiting example of an unnatural amino acid that may be incorporated to provide a unique attachment point is the biotinylated unnatural amino acid biocytin. The protein may then be tethered to or immobilized on the optical interface using the high-affinity biotin-streptavidin interaction to tether the protein to streptavidin molecules immobilized on the substrate surface. Other examples of suitable tethering or attachment techniques will be discussed in more detail below.

In some preferred embodiments, the protein for which tertiary structure is to be monitored may be a protein that has been genetically-engineered to incorporate both a unique nonlinear-active labeling site or nonlinear-active amino acid residue, and a unique tethering or immobilization site.

Measurement of protein structural stability: In some embodiments, the disclosed methods, devices, and systems may be used for monitoring protein stability (e.g., for biologics, biological protein-based drugs, or other proteins of interest) over time and/or under different experimental conditions (e.g., in different buffers, in different storage conditions, different attachment linkers, etc.) through the use of nonlinear optical techniques to monitor protein conformation, protein orientational distribution, or changes thereof. For example, in some embodiments, the protein molecule (e.g., a monoclonal antibody (mAb) drug or drug candidate) may be labeled with an SHG-active or other nonlinear-active label and tethered to an optical interface by any of a variety of means known to those of skill in the art. Changes in SHG or other nonlinear optical signal intensities (or other physical properties of nonlinear optical signals) arising from the labeled protein upon illumination by light of a fundamental frequency would then be monitored as a function of time, or upon contacting the labeled protein with one or more candidate stabilization compounds, candidate disruptive compounds, candidate stabilization or storage buffers, etc. In a preferred embodiment, ratios of SHG-to-TPF signal intensities (or ratios of other physical properties of nonlinear optical signals) may be used for monitoring the protein sample as a function of time, or upon contacting the labeled protein with one or more ligands, candidate stabilization compounds, candidate disruptive compounds, candidate stabilization compounds, temperatures, buffers, etc. Comparison of the resulting SHG or nonlinear optical signals or signal ratios (i.e., “signatures”) then provides a means for monitoring protein conformation, orientational distribution, or changes thereof, and establishing the stability of the protein as a function of time under different experimental conditions. In some embodiments, the stability of the protein may be determined under different physical conditions (e.g. at different temperatures) as well as under the different chemical conditions (e.g., different pH, different ionic strengths, different buffers, etc.). The stability determination may be based on monitoring changes in SHG or other nonlinear optical signals or signal ratios in real-time (e.g., on the basis of kinetic measurements), or may be based on end-point measurements of the SHG or other nonlinear optical signals or signal ratios.

Thus, the baseline SHG signal or (SHG-to-TPF signal ratio) provides a relative measure of protein stability over time or under different sets of experimental conditions. More disordered protein may lead to lower signal (and the converse) because the width of the orientational distribution for the tethered protein, but not necessarily the mean angle, changes in destabilizing buffers. The SHG signal intensity is proportional to <cos³ ϕ>², where ϕ is the angle between the molecular hyperpolarizability axis (assumed to be a single, dominant tensor element) and the normal to the surface in the label molecule frame of reference, and where the brackets denote an orientational average. A less stable and thus more unfolded protein should produce a wider orientational distribution and less SHG signal. In the limit where the protein is fully denatured, the signal should approach or be equal to zero since the nonlinear-active dye should approach or be fully randomly oriented with respect to the optical interface. This prediction is borne out by the experimental data to be described in the attached example in which a nonlinear-active labeled, tethered Fab fragment was exposed to increasing concentrations of urea to generate a denaturation curve. One can also obtain estimates of the free energy of denaturation from such a curve.

In some embodiments, the disclosed methods may be used to monitor protein stability under a defined set of conditions, e.g., experimental conditions or storage conditions, or following a change in the defined set of conditions, on the timescale of seconds, minutes, hours, days, or weeks.

Protein protein interactions and screening for compounds that stabilize or disrupt protein protein complexes: In some embodiments, the disclosed methods devices and systems may be used to monitor protein-protein interactions and/or to screen for compounds that stabilize or disrupt protein-protein complexes. In some cases, the protein that is tethered on the optical interface may be labeled with a nonlinear-active label, and the binding of one or more additional protein molecules may be monitored by means of conformational changes induced in the tethered molecule, e.g., by measuring changes in baseline SHG signals or in SHG-to-TPF signal ratios upon contacting the tethered molecule with the one or more additional molecules. In some cases the protein that is tethered on the optical interface is not labeled, and the binding of one or more nonlinear-active labeled proteins to the tethered protein may be monitored by means of measuring changes in baseline SHG signals or in SHG-to-TPF signal ratios upon contacting the tethered molecule with the one or more additional molecules. In some cases, the one or more additional protein molecules may be the same as the tethered molecule. In some cases, the one or more additional protein molecules may be different from the tethered molecule or different from each other. In some cases, at least one of the one or more additional protein molecules may be a naturally-occurring ligand or binding partner for the tethered molecule. In cases where a non-tethered protein molecule (or naturally occurring ligand) is the nonlinear-active species, the binding of the nonlinear-active labeled protein (or ligand) to the tethered protein may be viewed as a form of in situ labeling of the tethered protein or protein-protein complex. In some cases the proteins or other biomolecules to be studied are unlabeled and not nonlinear-active but the substrates or molecular ligands that bind to them are nonlinear-active labeled, e.g., ATTO390 GTP: γ-(6-Aminohexyl)-GTP-ATTO-390 γ-(6-Aminohexyl)-guanosine-5′-triphosphate).

In some embodiments, the disclosed methods, devices, and systems, may be used to screen candidate compound libraries in order to identify compounds that either stabilize or disrupt the resulting protein-protein complexes formed on the optical interface as result of the protein-protein binding interactions described above, e.g., the disclosed methods, devices, and systems may be used to screen candidate compound libraries to identify compounds that either stabilize or disrupt the protein-protein complexes upon contacting the complexes with one or more candidate compounds. In some cases, such screening for compounds that either stabilize or disrupt protein-protein complexes may be performed in a high-throughput manner using the devices and systems described in more detail below.

Comparison of protein structure for two or more samples: In some embodiments, the disclosed nonlinear optical methods may be utilized for structural comparison of two or more protein samples (or other biomolecule samples), e.g., two or more protein samples produced at different times by the same manufacturing process, or two or more protein samples produced by two different manufacturing processes, or two or more protein samples produced by the same manufacturing process but that have subsequently been subjected to different experimental conditions. For example, in some embodiments, two or more samples of a protein molecule (e.g., a monoclonal antibody (mAb) drug or drug candidate) may be labeled with an SHG-active or other nonlinear-active label using identical labeling protocols and tethered to an optical interface using identical tethering protocols, and measurements of an SHG baseline signal (or SHG-to-TPF signal ratio) may be performed using the same optical instrument (at the same time or at different times provided that the optical instrument has been calibrated against a reliable reference standard). Comparison of the resulting baseline SHG signal or SHG-to-TPF signal ratios then provides a means for monitoring protein structure, conformation, orientational distribution, or differences thereof, and may be used to establish that the two protein samples comprise protein of the same or substantially the same structure and conformation. In some embodiments, the disclosed methods, devices, and systems may be used for structural comparison of at least two, at least three, at least four, at least five, at least ten, at least twenty, at least thirty, at least forty, at least fifty, at least one hundred, or more than one hundred protein samples produced at different times by the same manufacturing process. In some embodiments, the disclosed methods, devices, and systems may be used for structural comparison of at least two, at least three, at least four, at least five, at least ten, at least twenty, at least thirty, at least forty, at least fifty, at least one hundred, or more than one hundred protein samples produced by two or more different manufacturing processes. In some embodiments, the disclosed methods, devices, and systems may be used for structural comparison of at least two, at least three, at least four, at least five, at least ten, at least twenty, at least thirty, at least forty, at least fifty, at least one hundred, or more than one hundred protein samples produced by the same manufacturing process but that have subsequently been subjected to different experimental conditions

Process optimization and quality control: In some embodiments, the disclosed nonlinear optical methods may be utilized for process optimization and/or quality control purposes. In general, the methodology underlying the use of nonlinear optical techniques for process optimization and monitoring of process output (e.g., as in quality control applications or demonstration of biosimilarity) will involve: (i) the collection of one or more aliquots of protein (e.g., at different times for the same step of the process, at different steps in the process, from different runs of the same process (e.g., different production lots), or from different production processes that nominally produce the same protein product, (ii) labeling of the protein (if necessary) using a standardized labeling procedure, (iii) tethering or immobilization of the labeled protein on a standardized optical surface (e.g., the surface of a glass substrate which may further comprise any of a variety of surface treatments or modifications known to those of skill in the art) using a standardized tethering or immobilization procedure under a standardized set of experimental conditions (e.g., buffer pH, ionic strength, detergent concentration, temperature, etc.), (iv) placement of the optical substrate (which, in some embodiments, may be incorporated into a device comprising wells or chambers for containment of buffers, assay reagents, or other solutions) in an instrument configured to provide illumination at one or more fundamental frequencies of light, and to detect light arising from a nonlinear optical process as a result of the illumination, (v) measurement of the baseline SHG (or other nonlinear optical) signal (or of an SHG-to-TPF signal ratio), and (vi) comparison of the baseline SHG signal or SHG-to-TPF signal for the one or more sample aliquots to each other or to that for a reference sample. In some embodiments, the one or more protein samples may be incubated with a test compound prior to or after tethering to or immobilization on the substrate. In some embodiments, the one or more protein samples may be exposed to a different set of experimental conditions prior to or after tethering to or immobilization on the substrate. In some embodiments, the optical system used for measurement of baseline SHG signals further comprises a fluorescence detection channel that may be used to monitor intrinsic fluorescence of the protein or nonlinear-active label (or of an additional fluorescent label attached to the protein), and used to normalize for well-to-well (sample-to-sample) variation in the surface density of immobilized protein. The disclosed measurement techniques provide a relatively quick and easy approach to monitoring protein structural variation between samples compared to conventional structural characterization techniques, e.g., x-ray crystallographic studies. Furthermore, the disclosed measurement techniques provide an approach to monitor protein structural variation between samples in solution.

The method may be used for any application requiring one to monitor and/or confirm protein structural similarity for protein samples taken at repeated time intervals or at different process steps, or for protein samples subjected to different sets of experimental conditions. In some embodiments, the approach may include real-time monitoring of protein structural variation. In some embodiments, as noted above, the approach may be used for monitoring protein stability during, for example, optimization of buffer formulations. In these embodiments, the protein under study remains tethered to or immobilized on a substrate surface (i.e., a fixed parameter of the experiment) and SHG signals (or SHG-to-TPF signal ratios) are measured while other experimental conditions (e.g., buffer conditions) are manipulated to optimize protein stability. In other embodiments, e.g., for process optimization, one or more sample aliquots are collected at different time points or at different steps in a process (i.e., the protein samples are the variable parameter of the experiment), and baseline SHG signal measurements (or SHG-to-TPF signal ratio measurements) are used to assess protein tertiary structure under a standardized set of experimental conditions (e.g., buffer pH, ionic strength, detergent concentration, temperature, etc.). For example, the approach may be used to assess protein tertiary structure before and after performing a given process step (e.g., before and after a freezing or lyophilization step, or after each of one or more different steps in a purification process. In some embodiments, the approach may be used to monitor production process output, e.g., at a process endpoint for quality control purposes in the production of biologics. In these latter embodiments, protein structure is assessed and compared under an identical set of experimental conditions (i.e., the experimental conditions used for making the nonlinear optical signal measurements remain fixed, and the protein is manipulated between measurements).

Statistical design of experiments approach: In some embodiments of the present disclosure, e.g., the use of the disclosed nonlinear optical methods for monitoring protein stability and optimizing a buffer formulation, or for optimizing and monitoring a biological drug manufacturing process, the methods may be applied using a statistical design of experiments (SDOE) approach. SDOE allows one to perform complex optimization procedures by making experimental measurements for a minimal number of discrete experimental test conditions when the desired outcome (e.g., protein stability over a specified time period, or consistent biological drug production) constitutes a local maximum for a complex “response surface” that is a function of many different experimental input parameters (e.g., buffer pH, ionic strength, detergent concentration, additive concentration, process step duration, etc.).

Demonstration of biosimilarity: In some embodiments, the disclosed methods provide a means for direct comparison of the structure (or conformation) of biosimilar drug candidates and reference drugs. In some embodiments, the disclosed methods provide a means for direct comparison of conformational changes induced in biosimilar drug candidates and reference drugs upon contact with an agent that binds to the biosimilar drug candidate and the reference drug. In some embodiments, the disclosed methods provide a means for direct comparison of conformational changes induced in a target protein or other biological entity upon contact with the biosimilar drug candidate and the reference drug. Also disclosed herein are systems with which these methods may be implemented in a high throughput manner.

As noted above, the surface selective property of SHG and related nonlinear optical techniques (or SHG-to-TPF signal ratios) can be exploited to determine the average orientation of nonlinear-active moieties, and can thus be used to compare structural similarity or to detect conformational change in biological molecules tethered at interfaces. For example, the structural similarity of a biological (biosimilar) drug candidate and a reference drug may be performed by labeling the biological drug candidate and reference drug with a nonlinear-active moiety using identical labeling reactions, tethering the biological drug candidate and reference drug to an interface using identical tethering methods such that they have a net orientation at the interface, and measuring a physical property of light generated by the non-linear active label upon illumination with light of a fundamental frequency (e.g., by measuring a baseline signal) for each. In some embodiments, a baseline signal ratio, e.g., the ratio of SHG-to-TPF baseline signal intensities, may be measured and used for demonstrating structural similarity between a biological drug candidate and a reference drug. A statistically significant difference in the physical property of light, e.g., a baseline signal intensity or baseline signal intensity ratio, measured for the biosimilar drug candidate and the reference drug may indicate that they are not structurally equivalent, while a statistically insignificant difference in the physical property of measured light may indicate that they have substantially the same structure.

The surface selective property of SHG and other nonlinear optical techniques can also be exploited to detect conformational change in biological molecules tethered at interfaces, and thus may be used to further demonstrate biosimilarity. For example, conformational change in a target protein molecule due to binding of a ligand (e.g., a biological drug candidate or a reference drug), might be detected using a nonlinear-active label or moiety wherein the label is attached to or associated with the target protein such that the conformational change leads to a change in the orientation or distance of the label with respect to the interface (FIG. 3), and thus to a change in a physical property of the nonlinear optical signal. Demonstration that the target protein undergoes the same conformational change upon binding of the biological drug candidate or reference drug, as indicated by the resultant change in SHG signal (or SHG-to-TPF signal ratio), would thus provide evidence of biosimilarity.

The methods and systems disclosed herein provide a means for real-time structural comparison of biological drug candidates (e.g., monoclonal antibodies (mAb)) to reference biological drugs for the purposes of establishing biosimilarity. The disclosed methods and systems comprise the use of SHG and related nonlinear optical techniques to compare the structures or conformations of a nonlinear-active labeled biological drug candidate and reference drug, and to monitor protein conformational changes upon contacting a nonlinear-active labeled biological protein target molecule (e.g., an antigen in the case of mAb drugs or drug candidates) with one or more drug candidates or the reference drug, thereby allowing comparison of the resulting conformational changes (or “conformational signatures”) for the purpose of establishing their equivalence or difference. Observation of the same or substantially the same structures or conformations in an identically labeled and tethered drug candidate and reference drug may provide evidence of biosimilarity. Observation of the same or substantially the same conformational changes or signatures for a drug candidate and the reference drug may indicate similar mechanisms of action and effectiveness. Observation of different conformational changes or signatures for a drug candidate and the reference drug may indicate different mechanisms of action and/or different levels of effectiveness. Conformational changes of the target molecule may be monitored as a function of time in kinetic measurements of SHG signal intensity (or the ratio of SHG-to-TPF signal intensities), or may be monitored by means of end point measurements. The disclosed nonlinear optical assay techniques thus enable real-time measurement and comparison of structure for biological drug candidates and reference drugs, and real-time measurement and comparison of conformational change in biological targets that are induced upon contacting the target molecule with biological drug candidates or reference drugs.

The disclosed methods for comparing biological drug candidates (e.g., generic drug candidates) to reference drugs (e.g., branded drugs) may be more sensitive to structural/conformational differences than many of the structural characterization techniques that are currently in use, and may be performed in a variety of different formats. For example, in a first embodiment, one or more candidate biological drug molecules and the reference drug molecule may be labeled with an SHG-active or nonlinear-active label and tethered to an optical interface by any of a variety of means known to those of skill in the art. For full-length mAbs, for example, this could be accomplished by means of binding to Protein A or G molecules which are immobilized on the surface. The candidate drug molecule (e.g., the generic or biosimilar) and the reference drug molecule (e.g., the branded drug) should have identical baseline signals if they are structurally equivalent and have been labeled and tethered to the interface in the same way. If not, the difference in SHG or nonlinear optical signature would provide structural evidence of their difference. In some embodiments, the degree of structural similarity (or conversely, the degree of structural dissimilarity) may be assessed by determining the statistical significance of the difference, if any, between measurements of the baseline SHG signal (or other nonlinear optical signal) for the labeled biological drug candidate and reference drug. For example, in some embodiments, a p-value of less than 0.001, less than 0.005, less than 0.01, less than 0.02, less than 0.03, less than 0.04, or less than 0.05 for sets of baseline SHG signal measurements for the labeled biological drug candidate and reference drug may indicate that a difference in the measured baseline signals is significantly different, and that the biological drug candidate and reference drug are not structurally equivalent.

In a second embodiment, a the target molecule (e.g., the antigen for a mAb drug or biological drug candidate) may be labeled with an SHG-active or other nonlinear-active label and tethered to an optical interface by any of a variety of means known to those of skill in the art. Changes in SHG or other nonlinear optical signal intensities (or other physical properties of the nonlinear optical signal) arising from conformational changes induced in the target molecule may then be monitored upon contacting the labeled target with the one or more candidate biological drugs or the reference drug. Comparison of the resulting changes in SHG signal or SHG-to-TPF signal ratio (signatures) then provides a means for establishing the similarity of the drug candidates to the reference drug in terms of the binding interaction with and/or conformational change induced in the target molecule. For example, in some embodiments, the degree of structural similarity (or conversely, the degree of structural dissimilarity) between the drug candidates and the reference drug may be assessed by determining the statistical significance of the difference, if any, between the measured changes of the SHG signal (or SHG-to-TPF signal ratio) for the labeled target molecule upon contacting the target molecule with the biological drug candidate and reference drug. For example, in some embodiments, a p-value of less than 0.001, less than 0.005, less than 0.01, less than 0.02, less than 0.03, less than 0.04, or less than 0.05 for sets of measured changes in SHG signal (or SHG-to-TPF signal ratios) for the labeled target molecule may indicate that a difference in the measured signal change is significantly different, and that the biological drug candidate and reference drug are not structurally equivalent.

In a third embodiment, candidate drug molecules (e.g., mAb drug candidates) and the reference drug molecule (e.g., mAb drug) may be labeled with a nonlinear-active moiety and tethered to an optical interface, and an SHG signal (or SHG-to-TPF signal ratio) may be monitored as the tethered drug candidates and reference drug are subsequently be contacted with the biological target molecule (e.g., the antigen in the case of mAb drugs or drug candidates). Again, if the two drug molecules are identical (or substantially the same), contacting them with the target molecule (e.g., the antigen) should elicit identical conformational responses, as indicated by the corresponding changes in SHG signal or SHG-to-TPF signal ratio (signatures). For example, in some embodiments, the degree of structural similarity (or conversely, the degree of structural dissimilarity) between the drug candidates and the reference drug may be assessed by determining the statistical significance of the difference, if any, between the measured changes of the SHG signal (or SHG-to-TPF signal ratio) for the labeled drug candidate and reference drug upon contacting them with the target molecule. For example, in some embodiments, a p-value of less than 0.001, less than 0.005, less than 0.01, less than 0.02, less than 0.03, less than 0.04, or less than 0.05 for sets of measured changes in SHG signal (or SHG-to-TPF signal ratios) for the labeled biological drug candidate and reference drug may indicate that a difference in the measured signal change is significantly different, and that the biological drug candidate and reference drug are not structurally equivalent.

In a fourth embodiment, labeled biological drug candidates (e.g., mAb drug candidates) or a reference drug (e.g., a branded mAb drug) can be added to unlabeled target protein (e.g., an antigen) tethered to the surface. Binding should produce a net, average orientation of the label, and thus a baseline signal. If the drug candidate is identical to the branded biologic drug, both should produce the same baseline SHG or other nonlinear optical signal. In some embodiments, the degree of structural similarity (or conversely, the degree of structural dissimilarity) may be assessed by determining the statistical significance of the difference, if any, between measurements of the baseline SHG signal (or other nonlinear optical signal) for the labeled biological drug candidate and reference drug. For example, in some embodiments, a p-value of less than 0.001, less than 0.005, less than 0.01, less than 0.02, less than 0.03, less than 0.04, or less than 0.05 for sets of baseline SHG signal measurements for the labeled biological drug candidate and reference drug may indicate that a difference in the measured baseline signals is significantly different, and that the biological drug candidate and reference drug are not structurally equivalent.

In any of the embodiments disclosed above, the method for establishing structural equivalence may further comprise simultaneous or serial measurement of a two-photon fluorescence (TPF) signal, and its use for calculating an SHG:TPF signal ratio (or an SFG:TPF signal ratio or DFG:TPF signal ratio) for the purpose of normalizing the measured nonlinear signal to the number of molecules tethered per unit area of the interface (i.e., the surface density or number density of tethered molecules on the interface). For example, in some embodiments, the nonlinear-active (i.e., SHG-active, SFG-active, or DFG-active) label used to label biological drug candidates and a reference drug prior to tethering them to an interface may also produce two-photon fluorescence when illuminated with light of a fundamental frequency that is the same as or different than that used to generate second harmonic, sum frequency, or difference frequency light. In some embodiments, the biological drug candidate(s) and reference drug may be labeled with a two-photon fluorescent label that is different than the SHG-active, SFG-active, or DFG-active label. Because the two-photon fluorescence signal is linearly related to the number of labeled molecules being excited, the two-photon fluorescence signal provides a means for normalizing the SHG (or SFG or DFG) signal to correct for variations in the surface density of the tethered molecules. In some embodiments, the two-photon fluorescence may be excited by delivery of the fundamental light (i.e., the excitation light, which is typically provided by a laser) to the interface using total internal reflection. In some embodiments, the two-photon fluorescence may be excited by delivery of the fundamental light in a direction that is orthogonal to the plane of the interface (e.g., using an epifluorescence optical setup), or at an arbitrary angle that is not orthogonal to the plane of the interface. In some embodiments, the two-photon fluorescence that is excited upon illumination with the fundamental frequency light may be detected and measured using an epifluorescence optical setup, e.g., wherein the emitted two-photon fluorescence is collected using a microscope objective. In some embodiments, the two-photon fluorescence may be detected and measured using a low-NA pinhole (i.e., without the use of a lens) positioned either directly above or below the point at which the excitation light is focused and oriented such that it is parallel to the plane of the interface. Two-photon fluorescence light passing through a collection lens, a microscope objective, or a pinhole (and any intermediate optical elements such as additional lenses, mirrors, dichroic reflectors, bandpass filters, and/or apertures) may then be detected using a photomultiplier or other suitable detector. PyMPO dye and analogs are suitable TPF dyes, for example PyMPO maleimide which is 1-(2-maleimidylethyl)-4-(5-(4-methoxyphenyl) oxazol-2-yl)pyridinium methanesulfonate.

Establishing a biosimilar “fingerprint”: The disclosed methods and systems may thus be used for establishing the biosimilarity of biological drug candidates (i.e., biosimilar drug candidates) to reference drugs targeting any of a variety of therapeutic targets. A number of recent publications have stressed the requirement for the use of a variety of orthogonal structural and functional characterization techniques and the collection of “fingerprint-like” comparative data sets to demonstrate complete biosimilarity (see, e.g., Greer, (2016), “Biosimilar Breakdown”, The Analytical Scientist, Issue 0916-401; and Declerck, (2013), “Biosimilar Monoclonal Antibodies: A Science-Based Regulatory Challenge”, Expert Opin. Biol. Ther. 13(2):153-156). The concept of a biosimilar “fingerprint” was introduced by the FDA to ensure that biosimilar drug developers give careful consideration of the techniques used to demonstrate equivalence of the biosimilar and reference drug.

Both clinical and non-clinical data are used to demonstrate biosimilarity, and the techniques employed for structural and functional characterization will typically vary from one biosimilar to another. For example, in attempting to demonstrate biosimilarity for therapeutic monoclonal antibodies (mAbs), it is important to realize that they comprise multiple peptide domains that contribute to their mode of action and affect their clinical properties (Declerck, (2013)). The Fab region contains the variable peptide domains responsible for the specific binding interactions with the target. The Fc region plays an important role in antibody-dependent cell-mediated cytotoxicity (ADCC), in complement-dependent cytotoxicity (CDC), and may exert other general regulatory effects on cell cycling by triggering signaling pathways. The Fc region is glycosylated, and the type and extent of glycosylation impact both effector function and clearance rate. The Fab region is also glycosylated and its potential impact on function should not be ignored. Therefore, evaluation of biosimilar mAbs should include not only characterization of Fab-mediated antigen-binding but also Fc-mediated functions (e.g., binding to FcγR, FcRn, complement). Characterization of Fab-associated functions should not be restricted to determination of antigen binding but should also include testing of the expected functional effects on the target (e.g., neutralization, receptor blocking, and receptor activation). Because of this complexity, demonstrating biosimilarity of mAbs may require not only in vitro structural/functional evaluation but also extensive in vivo functional evaluation. According to guidelines issued by the European Medicines Agency (Guideline on Similar Biological Medicinal Products Containing Monoclonal Antibodies—Non-clinical and Clinical Issues, 2012), a first step in demonstrating mAb biosimilarity comprises the evaluation of particular binding and functional characteristics. In vitro characterization studies are required in which the biosimilar and the reference are compared to each other with respect to: (i) binding to the target antigen, (ii) binding to representative isoforms of the relevant three Fc gamma receptors (FcγRI, FcγRII, and FcγRIII), FcRn and complement (C1q), (iii) Fab-associated functions (e.g., neutralization of a soluble ligand, receptor activation or blockade) and (iv) Fc-associated functions (e.g., ADCC, CDC, and complement activation). The need for in vivo non-clinical testing is based on an evaluation of the in vitro characterization data and the extent to which relevant structural differences (e.g., new post-translational modifications) or functional differences (e.g., changes in binding affinities, Fab-associated functions, or Fc-associated functions) between the mAb drug candidate and reference drug have been identified. If critical differences have been identified in the in vitro characterization data, then relevant animal model studies may be warranted.

Examples of structural characterization data that may be required to establish a biosimilar fingerprint may include, for example, primary structure (such as amino acid sequence determined by mass spectrometry or by performing nucleic acid sequencing), higher order structure (including secondary, tertiary, and quaternary structure (including aggregation)), enzymatic posttranslational modifications (such as glycosylation and phosphorylation), and other potential structural variations (such as protein deamidation and oxidation). Structural characterization of intentional chemical modifications (such as PEGylation sites and characteristics) may also be required.

In addition to their use in establishing biosimilarity, the disclosed methods and systems may also be used for establishing quality specifications for biosimilar drugs. ICH Topic Q6B is a guideline from the International Conference on Harmonisation of Technical Requirements for Registration of Pharmaceuticals for Human Use that defines test procedures for setting quality specifications for biological drug products (Greer, (2016), “Biosimilar Breakdown”, The Analytical Scientist, Issue 0916-401). Six specification requirements for structural characterization of biosimilar drugs are mentioned: (i) amino acid sequence, (ii) amino acid composition, (iii) terminal amino acid sequences, (iv) peptide map, (v) sulfhydryl group(s) and disulfide bridges, and (vi) carbohydrate structure (if appropriate). There are also six specification requirements for physicochemical characterization of biosimilar drugs: (i) molecular weight or size, (ii) isoform pattern, (iii) extinction coefficient, (iv) electrophoretic pattern, (v) liquid chromatographic pattern, and (vi) spectroscopic profiles. Examples of the different characterization techniques and tools that are currently used to determine structural and/or physicochemical properties of biosimilar drug candidates and drugs are listed in Table 2. Non-limiting examples of property determinations for which the presently disclosed nonlinear optical methods may be applicable are indicated in the table.

TABLE 2
Potential Methods for Demonstrating Biosimilarity (adapted from Greer (2016).

| Property To Be Determined | Characterization Methods |
|---|---|
| Amino acid sequence and modifications thereof | Mass spectrometry, peptide mapping, chromatography |
| Glycosylation | Anion exchange, enzymatic digestion, peptide mapping, |
| | capillary electrophoresis, mass spectrometry |
| Folding | Mass spectrometry S-S bridge determination, |
| | calorimetry, hydrogen deuterium exchange and ion |
| | mobility mass spectrometry, nuclear magnetic resonance, |
| | circular dichroism, Fourier transform spectroscopy, |
| | fluorescence, nonlinear optical techniques |
| PEGylation and isomerization | Chromatography, peptide mapping |
| Aggregation | Analytical ultracentrifugation, size-exclusion |
| | chromatography, asymmetric field flow fractionation, |
| | dynamic light scattering, microscopy, transmission |
| | electron microscopy, nonlinear optical techniques |
| Proteolysis | Electrophoresis, chromatography, mass spectrometry |
| Impurities | Proteomics, immunoassays, metal and solvents analysis |
| Subunit interactions | Chromatography, ion mobility mas spectrometry, |
| | nonlinear optical techniques |
| Heterogeneity of size, charge, hydrophobicity | Chromatography, gel and capillary electrophoresis, light |
| | scattering, ion mobility-mass spectrometry, capillary |
| | electrophoresis-mass spectrometry, nonlinear optical |
| | techniques |

The pharmacologic activity (e.g., the effectiveness, potency, and rate of occurrence of negative side effects) of biological drug candidates may be evaluated using a variety of in vitro and/or in vivo functional assays known to those of skill in the art. Examples of in vitro assays that may be used include, but are not limited to, biological assays, binding assays, enzymatic assays, and cell-based assays (e.g., cell proliferation assays or cell-based reporter assays). Examples of in vivo assays may include the use of animal model studies using animal models of disease (e.g., models that exhibit a disease state or symptom) to evaluate the functional effects of the candidate drug on pharmacodynamic markers or efficacy measures. A functional evaluation comparing the candidate drug to the reference drug using these functional assay data is an important part of the demonstration of biosimilarity, and may further be used to scientifically justify a selective and targeted approach to animal and/or clinical studies with human patients.

Thus, in some embodiments, the disclosed methods for comparison of biological drug candidates and reference drugs using a nonlinear optical measurement of protein structure and conformational signatures may be paired with other structural and/or functional assay techniques to provide a more complete characterization of biosimilarity. In these embodiments, the nonlinear optical characterization and comparison of candidate biological drugs and reference drugs may be performed in parallel with or in series with other structural or functional assays, as outlined above, and may provide for comparison of the candidate and reference drugs on the basis of structural equivalence, conformational signatures and potency (e.g., the magnitude of a response as a function of drug concentration), binding affinity, binding specificity, reaction kinetics, other structural characterization data (e.g., circular dichroism or crystallographic data), impact on intracellular signaling pathways and/or gene expression profiles, and the like. In some embodiments, the drug candidates (or generic drugs) and reference drugs (or branded drugs) may appear to be similar on the basis of one or more structural and/or functional characteristics, but may appear to differ on the basis of one or more different structural and/or functional characteristics, and the methods of the present disclosure may allow one to confirm or disprove biosimilarity.

Use of the disclosed methods, devices, and systems for structural comparisons of two or more samples, process optimization, process monitoring, or quality control purposes will require establishment not only of standardized labeling, tethering or immobilization, and measurement protocols, but also the development of criteria by which the baseline SHG signals (or SHG-to-TPF signal ratios) for protein samples collected at different time points, at different steps of a process, or from different protein lots are to be compared and judged “equivalent”. In many cases, the criteria may be protein-specific and will likely need to be established by performing comparison studies that utilize both the SHG baseline signal measurement techniques (or SHG-to-TPF signal ratio measurement techniques) disclosed herein and other structural or functional characterization methods.

In some embodiments, for example, it may be necessary to specify a range for the acceptable variation in baseline SHG signal (or baseline SHG-to-TPG signal ratio) that is required in order to conclude that protein samples collected at different times, or after different steps in a process, or from different lots of protein have equivalent structure. In some embodiments, the maximum allowable variation in baseline SHG signal (or other nonlinear optical signal) required to conclude that two or more protein samples have equivalent structure may range from about 0.1% to about 10%. In some embodiments, the maximum allowable variation in baseline SHG signal (or SHG-to-TPF signal ratio) may be at least 0.1%, at least 0.25%, at least 0.5%, at least 1%, at least 2%, at least 3%, at least 4%, at least 5%, at least 6%, at least 7%, at least 8%, at least 9%, or at least 10%. In some embodiments, the maximum allowable variation in baseline SHG signal (or SHG-to-TPF signal ratio) may be at most 10%, at most 9%, at most 8%, at most 7%, at most 6%, at most 5%, at most 4%, at most 3%, at most 2%, at most 1%, at most 0.5%, at most 0.25%, or at most 0.1%. Any of the lower and upper values described in this paragraph may be combined to form a range included within the disclosure, for example, the maximum allowable variation in baseline SHG signal (or SHG-to-TPF signal ratio) may range from about 2% to about 6%. Those of skill in the art will recognize that the maximum allowable variation in baseline SHG signal (or SHG-to-TPF signal ratio) may have any value within this range, e.g., about 4.5%.

In some embodiments, as another example, it may be necessary to require that the allowable amount of time elapsed between collection of two or more protein samples to be compared range from about 1 minute to about 1 week. In some embodiments, the allowable amount of time elapsed between collection of two or more protein samples to be compared may be at least 1 minute, at least 10 minutes, at least 20 minutes, at least 30 minutes, at least 40 minutes, at least 50 minutes, at least 1 hour, at least 2 hours, at least 3 hours, at least 4 hours, at least 5 hours, at least 6 hours, at least 12 hours, at least 18 hours, at least 1 day, at least 2 days, at least 3 days, at least 4 days, at least 5 days, at least 6 days, or at least 7 days. In some embodiments, the allowable amount of time elapsed between collection of two or more protein samples to be compared may be at most 7 days, at most 6 days, at most 5 days, at most 4 days, at most 3 days, at most 2 days, at most 1 day, at most 18 hours, at most 12 hours, at most 6 hours, at most 5 hours, at most 4 hours, at most 3 hours, at most 2 hours, at most 1 hour, at most 50 minutes, at most 40 minutes, at most 30 minutes, at most 20 minutes, at most 10 minutes, or at most 1 minute. Any of the lower and upper values described in this paragraph may be combined to form a range included within the disclosure, for example, the allowable amount of time elapsed between collection of two or more protein samples to be compared may range from about 10 minutes to about 2 hours. Those of skill in the art will recognize that the allowable amount of time elapsed between collection of two or more protein samples to be compared may have any value within this range, e.g., about 45 minutes.

In some embodiments, the maximum amount of time elapsed between collection of the protein sample and performance of the baseline SHG signal measurement (or other baseline nonlinear optical signal measurement, e.g., SHG-to-TPF signal ratio) may range from about 10 minutes to about 8 hours. In some embodiments, the maximum amount of time elapsed between collection of the protein sample and performance of the baseline SHG signal measurement (or SHG-to-TPF signal ratio measurement) may be at least 10 minutes, at least 20 minutes, at least 30 minutes, at least 40 minutes, at least 50 minutes, at least 1 hour, at least 2 hours, at least 3 hours, at least 4 hours, at least 5 hours, at least 6 hours, at least 7 hours, or at least 8 hours. In some embodiments, the maximum amount of time elapsed between collection of the protein sample and performance of the baseline SHG signal measurement (or SHG-to-TPF signal ratio measurement) may be at most 8 hours, at most 7 hours, at most 6 hours, at most 5 hours, at most 4 hours, at most 3 hours, at most 2 hours, at most 1 hour, at most 50 minutes, at most 40 minutes, at most 30 minutes, at most 20 minutes, or at most 10 minutes. Any of the lower and upper values described in this paragraph may be combined to form a range included within the disclosure, for example, the maximum amount of time elapsed between collection of the protein sample and performance of the baseline SHG signal measurement (or SHG-to-TPF signal ratio measurement) may range from about 20 minutes to about 2 hours. Those of skill in the art will recognize that the maximum amount of time elapsed between collection of the protein sample and performance of the baseline SHG signal measurement (or SHG-to-TPF signal ratio measurement) may have any value within this range, e.g., about 2.5 hours.

In some embodiments, the number or replicate measurements required for each protein sample to obtain a reliable comparison of two or more protein samples may range from about 1 replicate to about 6 replicates. In some embodiments, the number or replicate measurements required for each protein sample may be at least 1 replicate, at least 2 replicates, at least 3 replicates, at least 4 replicates, at least 5 replicates, or at least 6 replicates. In some embodiments, the number or replicate measurements required for each protein sample may be at most 6 replicates, at most 5 replicates, at most 4 replicates, at most 3 replicates, at most 2 replicates, or at most 1 replicate. Any of the lower and upper values described in this paragraph may be combined to form a range included within the disclosure, for example, the number or replicate measurements required for each protein sample may range from about 2 replicates to about 4 replicates. Those of skill in the art will recognize that the number or replicate measurements required for each protein sample may have any value within this range, e.g., 3 replicates.

As mentioned above, in many cases the criteria for establishing structural equivalence between two or more samples may be protein-specific, and will likely need to be established by performing comparison studies that utilize both the SHG baseline signal measurement techniques disclosed herein and other structural or functional characterization methods. Examples of suitable structural characterization techniques that may be performed in combination with the disclosed nonlinear optical measurement techniques include, but are not limited to, circular dichroism studies, nuclear magnetic resonance (NMR) studies, x-ray crystallography studies, molecular modeling studies, and the like. Examples of suitable functional characterization studies include, but are not limited to, ligand binding assays, enzymatic assays, immunoassays, and the like.

Other types of biological interactions detected: In addition to determining or comparing orientation or structure of proteins and other biological molecules, the methods and systems disclosed herein provide for detection of a variety of interactions between biological entities, or between biological entities and test entities, depending on the choice of biological entities, test entities, and non-linear active labeling technique employed. In one aspect, the present disclosure provides for the qualitative detection of binding events, e.g. the binding of a ligand to a receptor, as indicated by the resulting conformational change induced in the receptor. In another aspect, the present disclosure provides for quantitative analysis of binding events, e.g. the binding of a ligand to a receptor, by performing replicate measurements using different concentrations of the ligand molecule and generating a dose-response curve using the percent change in maximal conformational change observed. Similarly, other aspects of the present disclosure may provide methods for qualitative or quantitative measurements of enzyme-inhibitor interactions, antibody-antigen interactions, the formation of complexes of biological macromolecules, interactions of receptors with allosteric modulators, candidate drug-drug target interactions, protein-protein interactions, peripheral membrane protein-peripheral membrane protein interactions, peripheral-membrane protein-integral membrane protein interactions, peripheral membrane protein-phospholipid bilayer interactions, etc.

Interactions between biological entities or biological and test entities (e.g. binding reactions, conformational changes, etc.) can be correlated through the methods presently disclosed to the following measurable nonlinear signal parameters: (i) the intensity of the nonlinear light, (ii) the wavelength or spectrum of the nonlinear light, (iii) the polarization of the nonlinear light, (iv) the time-course of (i), (ii), or (iii), and/or vi) one or more combinations of (i), (ii), (iii), and (iv), as well as through measurement of signal ratios, e.g., SHG-to-TPF signal intensity ratios.

Absolute polar orientation determination: Although the tilt angle orientation of the label in the lab frame can be determined, this tilt angle is degenerate in two cones pointing toward and away from the surface, respectively. The present invention also discloses a novel method for obtaining the absolute direction of the labels, i.e. which direction the label points relative to the surface plane using a simple experiment. In this experiment, the SHG signal under a given polarization condition is measured using: i) labeled protein attached to an unlabeled surface; ii) unlabeled protein attached to a labeled surface and iii) labeled protein attached to a labeled surface. The labeled surface can be prepared in a variety of ways known to those skilled in the art, for example through covalent carbodiimide coupling of a carboxylated nonlinear-active label to an aminosilane-functionalized glass substrate surface. Alternatively, with supported lipid bilayers (SLBs), one can covalently couple the same nonlinear-active label that attaches via amines or thiols (e.g. corresponding to lysine or cysteine residue side-chains) to a protein to the bilayer doped with varying mole percentages of an amine or thiol-containing lipid. This surface then provides an SHG signal of its own which, because the label is the same as that of the protein label, is in phase with the SHG signal generated from the protein. The label attached to the supported bilayer has a known polar orientation by virtue of its known directional coupling to the surface and its chemical structure. An experiment to determine the absolute polar orientation of a label on a protein (and therefore potentially the polar orientation of the entire protein) can be carried out as follows. First, the SHG signal of the labeled surface in the absence of protein is measured (I_{L}). Second, the SHG signal of the labeled protein attached to the unlabeled surface is measured (I_{P}). Third, the SHG signal of the surface is measured when labeled protein is attached to the labeled surface (I_{TOT}). The relationship between the different SHG signal is as follows:

I_{TOT}=I_{L}+I_{P}+2*sqrt(I_{L}*I_{P})*cos(θ) (7)

where cos(θ) describes the phase relationship (which flips in sign with the absolute polar orientation toward or away from the surface) between the labels attached to the protein molecules and the surface in the third measurement (I_{TOT}). By measuring I_{TOT}, I_{L}, and I_{P} separately and comparing the measured signal intensities, the absolute polar orientation of each label on the protein can be determined. In some cases, e.g., when I_{L}+I_{P} are of roughly comparable magnitude, if I_{TOT} is smaller than I_{L} on its own, one can immediately determine that destructive interference is occurring between the protein label and the surface label; therefore, the labels are oriented in opposite polar orientations; if I_{TOT} is larger than I_{L} on its own, constructive interference is occurring and the labels are oriented in the same polar direction. The magnitude of I_{L}+I_{P} can be varied by tuning, for example, the density of attachment sites on the supported bilayer for the dye, or the density of proteins attached to the surface.

In a similar fashion, the phase difference between a dye probe attached to a biomolecule and the background in the absence of the biomolecule can be determined by arranging different proportions of labeled and unlabeled biomolecule in different measurements while keeping the total concentration of biomolecule constant across each measurement. In this way, interference between the background and the nonlinear-active probe will produce an intensity that depends on the phase difference between the SHG waves generated by the background and the dye probe. This can be accomplished most simply, for example, by incubating the same total protein concentration in the well, but varying the proportion of labeled and unlabeled protein. Each well should exhibit the same surface density of protein but the proportion of labeled and unlabeled molecules will be reflective of their concentration ratio during incubation. The total measured intensity I_{TOT} should thus depend on the phase difference between the SHG wave generated from the background signal (e.g., surface+water+unlabeled protein) and the labeled protein signal. By performing successive experiments at different ratios of labeled and unlabeled protein and measuring I_{TOT}, the relative orientation of the dye probe can be obtained by determining if the interference between the background and dye label is constructive or destructive using equation (7).

In some embodiments, the nonlinear-active labeled surface is prepared using covalent carbodiimide coupling of a carboxylated nonlinear-active label to an aminosilane-functionalized glass substrate surface. In some embodiments, the nonlinear-active labeled surface comprises a supported lipid bilayer, and wherein the supported lipid bilayer further comprises an amine- or thiol-containing lipid to which a nonlinear-active label is covalently coupled.

In some embodiments, the nonlinear-active labeled protein is tethered in an oriented fashion on the non-labeled or nonlinear-active labeled surface using covalent carbodiimide coupling of the C-terminus of the protein to an aminosilane-functionalized glass substrate surface. In some embodiments, the nonlinear-active labeled protein is tethered in an oriented fashion on a non-labeled or nonlinear-active labeled surface comprising a supported lipid bilayer, and wherein the nonlinear-active labeled protein is inserted into the supported lipid bilayer or attached to an anchor molecule that is inserted into the supported lipid bilayer. Methods for tethering labeled proteins and other biomolecules to substrate surfaces or supported lipid bilayers will be described in more detail below.

In summary, the disclosed methods for determining the absolute orientation of a nonlinear-active label attached to a tethered protein may comprise: (a) detecting a physical property of light generated by a nonlinear-active surface as a result of illumination with excitation light of at least one fundamental frequency, wherein detection is performed using two different polarization states of the excitation light; (b) detecting a physical property of light generated by a nonlinear-active labeled protein tethered in an oriented fashion on a non-labeled surface, wherein the light is generated as a result of illumination with excitation light of the at least one fundamental frequency, and wherein detection is performed using two different polarization states of the excitation light; (c) detecting a physical property of light generated by a nonlinear-active labeled protein tethered in an oriented fashion on a nonlinear-active labeled surface, wherein the light is generated as a result of illumination with excitation light of the at least one fundamental frequency, and wherein detection is performed using the two different polarization states of the excitation light; and (d) determining the absolute orientation of the nonlinear-active label attached to the tethered protein by comparing the physical property of the light in step (a), the physical property of the light detected in step (b), and the physical property of the light detected in step (c). If the orientational width is assumed or known to be narrow, one can use the ratio of TPF intensities measured under two orthogonal polarization states (e.g., s- and p-polarized) to determine the orientation of the label, i.e., the angle between the transition dipole moment and the normal axis to the surface. Likewise, the angle between the dominant hyperpolarizability component and the normal axis in a probe can be determined by taking the ratio of SHG intensities under two orthogonal polarizations. For simultaneous determination of the distribution width and the mean angle, one solves for the crossing point of the two mean angle, width trajectories that satisfy each ratio of intensities (TPF and SHG under p- and s-polarization, respectively) separately as shown in the example below.

Electric field orientation, strength and characteristics: In some embodiments, an electric field can be applied to manipulate the orientation of the biomolecules in the lab frame at the interface. The electric field direction can be across the surface, perpendicular to it, or in general, at any angle relative to the surface plane. In one embodiment, one electrode is placed underneath a lipid bilayer membrane or other surface chemistry for protein attachment to the substrate, e.g. a glass substrate. A counter-electrode is placed above the substrate surface plane, for example at the top of the liquid in a sample well. In another embodiment, two or more electrodes are placed in the substrate surface plane and the electric field direction is parallel to the substrate-membrane interface.

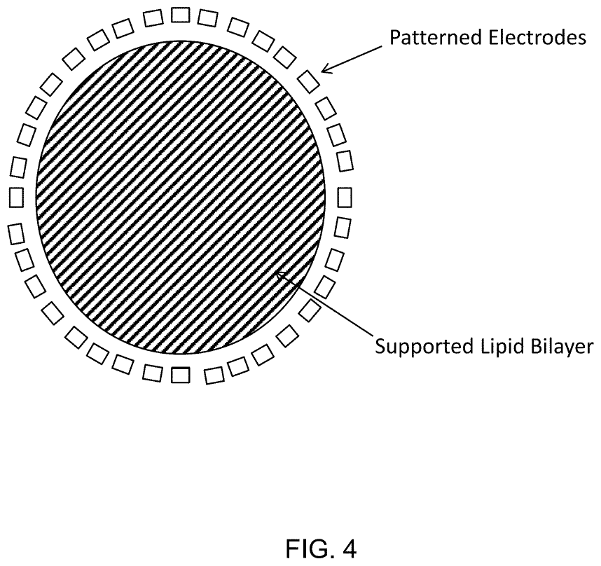

In another embodiment, an array of electrodes can be placed around the tethered or immobilized biomolecule, e.g., protein sample, as illustrated in FIG. 4. For example, a circular array of electrodes can be placed parallel to the membrane interface on a glass substrate surface, each spaced about 10 degrees apart from each other. Voltage applied to a pair of electrodes that are 180 degrees apart from each other allows the azimuthal direction of the electric field to be changed at will and in a rapid fashion. For example, the azimuthal direction of the electric field can be swept around the entire circle in a second or a fraction of a second.

Electrodes may be patterned on the substrate surface using any of a variety of techniques known to those of skill in the art. Examples include, but are not limited to, screen printing, photolithographic patterning, sputter coating, chemical vapor deposition, or any combination thereof.

Electrodes may be fabricated from any of a variety of material, as is well known to those of skill in the art. Examples of suitable electrode materials include, but are not limited to, silver, gold, platinum, copper, aluminum, graphite, indium tin oxide (ITO), semiconductor materials, conductive polymers, or any combination thereof.

In some embodiments, it may be desirable to passivate the surface of one or more electrodes, e.g., to minimize corrosion of the electrode surfaces that are in contact with aqueous buffers, and/or to prevent contamination of or interference with proteins or other biological components, and/or to prevent current flow in the sample. Any of a variety of passivation techniques known to those of skill in the art may be used, and will in general depend on the choice of materials used to fabricate the electrode(s). For example, indium tin oxide electrodes on glass substrates may be passivated by growth or deposition of a 30 nm SiO₂ layer. Metal or semiconductor electrodes will often develop an inert “native oxide” layer upon exposure to air that may serve as a passivation layer. This inert surface layer is usually an oxide or a nitride, with a thickness of a monolayer (1-3 Å) for platinum, about 15 Å for silicon, and may be close to 50 Å thick for aluminum after long exposures to air.

The electric field can be DC or AC, i.e. time-invariant or time-varying. In the latter case, it can take a sinusoidal wave of any frequency or it can be a complex wave (e.g., a step function, a saw tooth pattern, etc.) comprised of many frequency components, and the field can oscillate between positive or negative values or remain all positive or all negative. Non-periodic or pulsed electric fields can also be applied in some embodiments. The SHG signal (or ratio of SHG-to-TPF signals) can be read before, during or after application of an electric field to the sample.

In some embodiments, the electric field strength may range from about zero to about 10⁶ V/cm, or larger. In some embodiments, the electric field strength may be at least zero, at least 10 V/cm, at least 10² V/cm, at least 10³ V/cm, at least 10⁴ V/cm, at least 10⁵V/cm, or at least 10⁶ V/cm. In some embodiments, the electric field strength may be at most 10⁶ V/cm, at most 10⁵ V/cm, at most 10⁴ V/cm, at most 10³ V/cm, at most 10² V/cm, at most 10 V/cm. Those of skill in the art will recognize that the electric field strength may have any value within this range, for example, about 500 V/cm.

In some embodiments, the frequency at which the electric field is varied may range from about 0 Hz to about 10⁵ Hz. In some embodiments, the frequency at which the electric field is varied may be at least 0 Hz, at least 10 Hz, at least 10² Hz, at least 10³ Hz, at least 10⁴ Hz, or at least 10⁵ Hz. In some embodiments, the frequency at which the electric field is varied may be at most 10⁵ Hz, at most 10⁴ Hz, at most 10³ Hz, at most 10² Hz, or at most 10 Hz. Those of skill in the art will recognize that the frequency at which the electric field is varied may have any value within this range, for example, about 125 Hz.

The electric field can be used to manipulate the orientation of the protein molecules, or other biomolecules, and thus the baseline SHG signal (or baseline SHG-to-TPF signal ratio) or SHG (or SHG-to-TPF) polarization dependence. In some embodiments, if orientational isotropy in the substrate surface plane (i.e. the XY plane) occurs in the absence of an applied electric field and is preserved when a field is applied, only two or three independent non-vanishing components of the nonlinear susceptibility (χ⁽²⁾) will exist. In other embodiments in which orientational anisotropy is present in the surface plane, either before, during or after application of an electric field, more than two or three independent, non-vanishing components of χ⁽²⁾ will exist, allowing for additional independent SHG measurements with different combinations of polarized fundamental and second-harmonic light. In some embodiments in which orientational anisotropy exists at the surface plane (e.g., at a lipid biomembrane to which labeled proteins are attached), multiple independent measurements of the χ⁽²⁾ can be made at different azimuthal angles. For example, if an electric field is applied parallel to the surface, and this causes a change in the orientational distribution of the protein molecules from isotropic in-plane to anisotropic in-plane, additional independent optical measurements can be made in many azimuthal directions relative to the direction of the applied electric field to determine the molecular orientational distribution.

Optical multiwell plate with integrated electrodes: As will be discussed in more detail below, in some embodiments the TPF and/or SHG measurements described herein are preferentially performed using a microwell plate format. In general, these devices comprise: (a) a substrate comprising a first surface that further comprises a plurality of discrete regions, wherein each discrete region further comprises a patterned array of electrodes and optionally, a supported lipid bilayer; and (b) a well-forming component bonded to or integrated with the first surface of the substrate so that each discrete region is contained within a single well. In some embodiments using a 384-well plate (or other microwell plate or multi-chamber formats), the electrodes can be patterned on the substrate surface inside of and adjacent to the walls of the wells, as part of a lid that is used to seal the wells, elsewhere on the substrate surface (which may be glass) within the wells, or anywhere that allows both application of voltage to produce an electric field on the sample and optical reading of the TPF and/or SHG signals.

In some embodiments, a plurality of the wells comprise supported lipid bilayers that further comprise a nonlinear-active labeled protein (or other biological entity). In some embodiments, the plurality of supported lipid bilayers further comprises a nonlinear-active protein that is the same for each. In some embodiments, the plurality of supported lipid bilayers further comprise two or more subsets of supported lipid bilayers (e.g., residing in two or more subsets of the plurality of wells), and wherein each subset of supported lipid bilayers comprises a different nonlinear-active protein. In some embodiments, the substrate is fabricated from an optically-transparent material selected from the group consisting of glass, fused-silica, polymer, or any combination thereof. In some embodiments, the patterned array of electrodes comprises an array of two or more electrodes patterned on the substrate surface surrounding the supported lipid bilayer. In some embodiments, the patterned array of electrodes comprises an array of two or more electrodes patterned on the walls of each well of the well-forming unit. In some embodiments, the patterned array of electrodes comprises at least one electrode patterned on a lid that seals each well. In some embodiments, the well-forming unit comprises 96 wells. In some embodiments, the well-forming unit comprises 384 wells. In some embodiments, the well-forming unit comprises 1,536 wells. In some embodiments, the device further comprises an array of prisms integrated with a second surface of the substrate and configured to deliver excitation light to the first surface of the substrate so that it is totally internally reflected from the first surface.

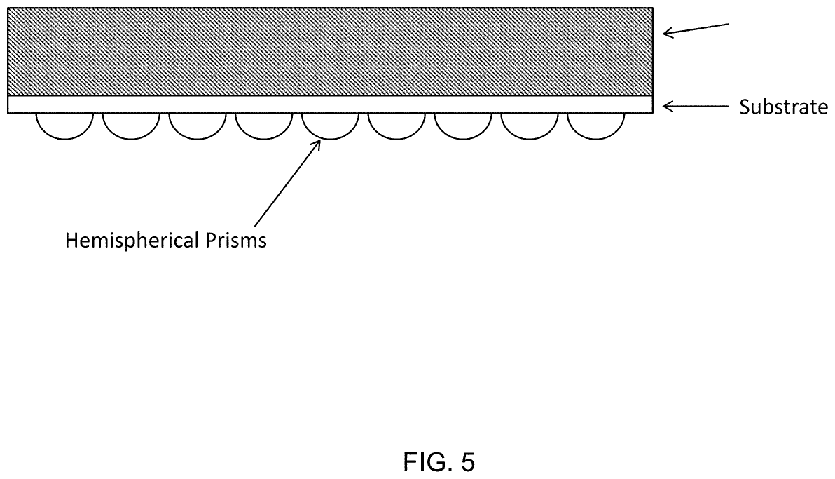

Optical multiwell plate with hemispherical prisms: In some embodiments, particularly in cases in which an anisotropic orientational distribution of the molecules exists at the surface, it will be useful to optically probe the sample at different azimuthal directions relative to the anisotropic axis. As an alternative to rotating the sample relative to the optical axis, the optical axis can be rotated relative to the fixed sample. To accomplish this, hemispherical prisms placed at the bottom of, or near each well, can be used to direct incoming light incident on the prisms at arbitrary angles relative to the well to the interfacial region containing the molecules. In some embodiments, the hemispherical prisms are bonded to or integrated with the substrate in a glass-bottom multi-well plate, as illustrated in FIG. 5. The hemispherical prisms make optical contact with the multi-well plate, thereby permitting transmission of the optical beam with minimal loss. In some embodiments, the optical multi-well plate will comprise a 384-well glass-bottom plate or other glass-bottom microwell plate format (i.e. standard microwell plate formats that are well known to those of skill in the art).

In some embodiments, the microwell plate device comprising an array of hemispherical prisms bonded to or integrated with the glass substrate that forms the bottom of the wells may further comprise a patterned array of electrodes on the upper surface of the glass substrate within each well so that polarized TPF and/or SHG measurements may be made while applying electric fields of different field strengths.

Measurement of conformation and ligand-induced conformational change: In one embodiment, a ligand-induced conformational change (e.g., a local conformational change) is measured at one or more label sites within the protein. In one embodiment, single-site cysteine residues are used. Combinations of polarized fundamental light and nonlinear light measurements are used to determine the components of χ⁽²⁾ before and after ligand addition. For example, if the biomolecules are oriented isotropically on the surface and the molecular hyperpolarizability is dominated by a single tensor element (e.g., α_{z′z′z′}, or equivalently in some literature β_{z′z′z′}) then intensity ratios for both SHG and TPF made under p- and s- (zzz and zxz) excitation polarization can be measured to yield two independent equations of ratios that depend only orientational angle (tilt angle ϕ relative the surface normal or z-axis) and the orientational distribution. This process can be repeated for any number of label sites using different single-site cysteine mutants, and a model of local or the global ligand-bound structure can be determined. In some embodiments, the model optionally incorporates the X-ray crystal structure coordinates or other structural constraints (e.g., from NMR data, small angle X-ray scattering data, or any other measurements known to those skilled in the art).

Determination of structural parameters and detection of conformational change under different experimental conditions: In some embodiments, the determination of structural parameters such as mean tilt angle or distribution width, and/or the detection of conformational change for labeled biological molecules using TPF measurements and/or SHG, SFG, or DFG measurements, or ratios thereof, may be facilitated by performing the measurements under two or more different sets of experimental conditions, where the two or more different sets of experimental conditions influence a structural parameter or conformation of the biomolecule.

As defined herein, “experimental conditions” refer to any set of experimental parameters under which SHG, TPF, and/or other nonlinear optical signals are measured, wherein a change in one or more of the experimental parameters in the set of experimental conditions results in a change in the measured values of TDM or χ⁽²⁾ due to a change in the underlying molecular orientational distribution. In other words, different sets of experimental conditions that produce different orientational distributions in the lab frame will produce different baseline TPF and/or SHG signal intensities, different polarization dependences, different responses to the same ligand binding event, or any or all of the aforementioned. In these experiments, we may assume the protein structure and conformational landscape remain constant and thus obtain a virtually unlimited number of independent data points from biomolecules labeled at two or more sites, globally oriented on the surface at different angles, for structure and conformational landscape determination. For example, applying an electric field to proteins attached to a supported lipid bilayer can change the molecules' underlying orientational distribution and thus may change the measured values of TDM or χ⁽²⁾. Other examples of experimental parameters that may be used to define sets of experimental conditions include, but are not limited to, buffer conditions such as pH, ionic strength, detergent content and concentration, tether attachment site (e.g., through the use of N- or C-terminal His tags), etc. Specifically, the independent values of TDM or χ⁽²⁾ measured under the different and independent sets of experimental conditions permit one to obtain the underlying molecular orientational distribution in the laboratory frame of reference (FIG. 2). By relating these different laboratory frame measurements to each other, one can determine the relative difference in angle between the nonlinear-active labels positioned at two or more different label sites in the protein frame of reference, along with other parameters of the orientational distribution such as the width of a gaussian distribution used to model the molecular orientational distribution.

In some embodiments, the use of one or more compositions of surfaces to which the labeled proteins are tethered may be used to define different sets of experimental conditions. For example, if supported lipid bilayers are used to tether and orient the labeled proteins, two or more different lipid compositions of the bilayer (for example, with different electrostatic charge densities or different molar lipid doping densities) can be used to create two or more different orientational distributions of the same protein. For example, lipids with head groups bearing different net charges can be used (e.g., zwitterionic, positively, and negatively charged lipid head groups). In some embodiments, it may be advantageous to vary the lipid composition of the supported lipid bilayer by varying, e.g., the number of different lipid components and/or their relative concentrations. Examples of lipid molecules that may be used to form supported lipid bilayers or that may be inserted as major or minor components of the supported lipid bilayer include, but are not limited to, diacylglycerol, phosphatidic acid (PA), phosphatidylethanolamine (PE), phosphatidylcholine (PC), phosphatidylserine (PS), phosphatidylinositol (PI), phosphatidylinositol phosphate (PIP), phosphatidylinositol biphosphate (PIP2), phosphatidylinositol triphosphate (PIP3), ceramide phosphorylcholine (sphingomyelin; SPH), ceramide phosphorylethanolamine (sphingomyelin; Cer-PE), ceramide phosphoryllipid, or any combination thereof. In some embodiments, a lipid molecule comprising a nickel-nitrilotriacetic acid chelate (Ni-NTA) moiety may be used for the purpose of tethering proteins by means of a His tag. For example, the bilayer may incorporate 1,2-dioleoyl-sn-glycero-3-[(N-(5-amino-1-carboxypentyl)iminodiacetic acid)succinyl] (nickel salt) at various molar concentrations.

In some embodiments, the number of different lipid components of the lipid bilayer may range from 1 to 10, or more. In some embodiments, the number of different lipid components may be at least 1, at least 2, at least 3, at least 4, at least 5, at least 6, at least 7, at least 8, at least 9, or at least 10. In some embodiments, the number of different lipid components may be at most 10, at most 9, at most 8, at most 7, at most 6, at most 5, at most 4, at most 3, at most 2, or at most 1.

In some embodiments, the relative percentage of a given lipid component of the lipid bilayer may range from about 0.1% to about 100%. In some embodiments, the relative percentage of a given lipid component may be at least about 0.1%, at least about 0.2%, at least about 0.3%, at least about 0.4%, at least about 0.5%, at least about 1%, at least about 5%, at least about 10%, at least about 20%, at least about 30%, at least about 40%, at least about 50%, at least about 60%, at least about 70%, at least about 80%, at least about 90%, or at least about 100%. In some embodiments, the relative percentage of a given lipid component may be at most about 100%, at most about 90%, at most about 80%, at most about 70%, at most about 60%, at most about 50%, at most about 40%, at most about 30%, at most about 20%, at most about 10%, at most about 5%, at most about 1%, at most about 0.5%, at most about 0.4%, at most about 0.3%, at most about 0.2%, or at most about 0.1%. Any of the lower and upper values described in this paragraph may be combined to form a range included within the present disclosure, for example, the relative percentage of a given lipid component of the lipid bilayer may range from about 0.5% to about 20%. Those of skill in the art will recognize that the relative percentage of a given lipid component in the lipid bilayer may have any value within this range, e.g. about 12.5%.

As noted above, in some embodiments, a first set of experimental conditions may comprise tethering protein molecules to a substrate surface or supported lipid bilayer using a His-tag attached to the N-terminus of the protein, and an at least second set of experimental conditions comprises tethering the protein molecules using a His-tag attached to the C-terminus. Attachment by means of N-terminal vs. C-terminal His-tags generally produces different orientational distributions, and thus may be used to define different sets of experimental conditions that yield different measured values of TDM or χ⁽²⁾. If tags such as His tags are used to tether the protein, different lengths of the His-tags may produce different orientational distributions, and therefore may be used to define different sets of experimental conditions. In some embodiments, the first set of experimental conditions may comprise tethering the protein molecules using a first His-tag selected from the group consisting of, for example, 2× His, 4× His, 6× His, 8× His, 10× His, 12× His, and 14× His, and the at least second set of experimental conditions may comprise tethering the protein molecules using an at least second His-tag that differs in length from the first His-tag.

In some embodiments, the length of the His tag used to tether a labeled protein to a supported lipid bilayer comprising a lipid having a Ni-NTA moiety attached may range from about 1 His residue to about 20 His residues, or more. In some embodiments, the length of the His tag may be at least 1 His residue, at least 2 His residues, at least 3 His residues, at least 4 His residues, at least 5 His residues, at least 6 His residues, at least 7 His residues, at least 8 His residues, at least 9 His residues, at least 10 His residues, at least 11 His residues, at least 12 His residues, at least 13 His residues, at least 14 His residues, at least 15 His residues, at least 16 His residues, at least 17 His residues, at least 18 His residues, at least 19 His residues, or at least 20 His residues. In some embodiments, the length of the His tag may be at most 20 His residues, at most 19 His residues, at most 18 His residues, at most 17 His residues, at most 16 His residues, at most 15 His residues, at most 14 His residues, at most 13 His residues, at most 12 His residues, at most 11 His residues, at most 10 His residues, at most 9 His residues, at most 8 His residues, at most 7 His residues, at most 6 His residues, at most 5 His residues, at most 4 His residues, at most 3 His residues, at most 2 His residues, or at most 1 His residue.

In some embodiments, the difference between a first buffer and at least a second buffer may be used to define different sets of experimental conditions. In some embodiments, the difference between buffers used to define different sets of experimental conditions may be selected from the group consisting of type of buffer, buffer pH, buffer viscosity, ionic strength, detergent concentration, zwitterionic component concentrations, calcium ion (Ca²⁺) concentration, magnesium ion (Mg²⁺) concentration, carbohydrates, bovine serum albumin (BSA), polyethylene glycol or other additive concentrations, antioxidants and reducing agents, or any combination thereof. Different buffer conditions may change the orientational distributions of the molecules and thus the measured values of TDM or χ⁽²⁾.

By way of example, suitable buffers for use in the disclosed methods may include, but are not limited to, phosphate buffered saline (PBS), succinate, citrate, histidine, acetate, Tris, TAPS, MOPS, PIPES, HEPES, MES, and the like. The choice of appropriate buffer will generally be dependent on the target pH of the buffer solution. In general, the desired pH of the buffer solution will range from about pH 6 to about pH 8.4. In some embodiments, the buffer pH may be at least 6.0, at least 6.2, at least 6.4, at least 6.6, at least 6.8, at least 7.0, at least 7.2, at least 7.4, at least 7.6, at least 7.8, at least 8.0, at least 8.2, or at least 8.4. In some embodiments, the buffer pH may be at most 8.4, at most 8.2, at most 8.0, at most 7.8, at most 7.6, at most 7.4, at most 7.2, at most 7.0, at most 6.8, at most 6.6, at most 6.4, at most 6.2, or at most 6.0. Any of the lower and upper values described in this paragraph may be combined to form a range included within the present disclosure, for example, the buffer pH may range from about 6.2 to about 8.2. Those of skill in the art will recognize that the buffer pH may have any value within this range, for example, about 7.25. In some cases, the pH of the buffer solution may range from about 4 to about 10.

In some embodiments, the ionic strength of the buffers used to define different sets of experimental conditions may comprise using monovalent salts (e.g. NaCl, KCl, etc.), divalent salts (e.g. CaCl₂, MgCl₂, etc.), trivalent salts (e.g. AlCl₃), or any combination thereof. In some embodiments, the ionic strength of the buffers used to define different sets of experimental conditions may range from about 0.0 M to about 1 M, or higher. In some embodiments, the ionic strength of the buffer may be at least 0.0 M, at least 0.1 M, at least 0.2 M, at least 0.3 M, at least 0.4 M, at least 0.5 M, at least 0.6 M, at least 0.7 M, at least 0.8 M, at least 0.9 M, or at least 1.0 M. In some embodiments, the ionic strength of the buffer may be at most 1.0 M, at most 0.9 M, at most 0.8 M, at most 0.7 M, at most 0.6 M, at most 0.5 M, at most 0.4 M, at most 0.3 M, at most 0.2 M, or at most 0.1 M. Any of the lower and upper values described in this paragraph may be combined to form a range included within the present disclosure, for example, the ionic strength of the buffer may range from about 0.4 M to about 0.8 M. Those of skill in the art will recognize that the ionic strength of the buffer may have any value within this range, for example, about 0.15 M.

Suitable detergents for use in buffer formulation include, but are not limited to, zitterionic detergents (e.g., 1-Dodecanoyl-sn-glycero-3-phosphocholine, 3-(4-tert-Butyl-1-pyridinio)-1-propanesulfonate, 3-(N,N-Dimethylmyristylammonio)propanesulfonate, 3-(N,N-Dimethylmyristylammonio)propanesulfonate, ASB-C80, C7BzO, CHAPS, CHAPS hydrate, CHAPSO, DDMAB, Dimethylethylammoniumpropane sulfonate, N,N-Dimethyldodecylamine N-oxide, N-Dodecyl-N,N-dimethyl-3-ammonio-1-propanesulfonate, or N-Dodecyl-N,N-dimethyl-3-ammonio-1-propanesulfonate) and anionic, cationic, and non-ionic detergents. Examples of nonionic detergents include poly(oxyethylene) ethers and related polymers (e.g. Brij®, TWEEN®, TRITON®, TRITON X-100 and IGEPAL® CA-630), bile salts, and glycosidic detergents.

In some embodiments, the concentration of detergent in the buffer may range from about 0.01% (w/v) to about 2% (w/v). In some embodiments, the concentration of detergent in the buffer may be at least 0.01%, at least 0.05%, at least 0.1%, at least 0.5%, at least 1.0%, at least 1.5%, or at least 2%. In some embodiments, the concentration of detergent in the buffer may be at most 2%, at most 1.5%, at most 1.0%, at most 0.5%, at most 0.1%, at most 0.05%, or at most 0.01%. Any of the lower and upper values described in this paragraph may be combined to form a range included within the present disclosure, for example, the concentration of detergent in the buffer may range from about 0.1% (w/v) to about 1.5% (w/v). Those of skill in the art will recognize that the concentration of detergent in the buffer may have any value within this range, for example, about 0.12% (w/v).

In some embodiments, buffer additives that associate with the interfacial region and produce different orientational distributions of the proteins as a function of their concentration can also be used, such as PEG400, ethylene glycol, etc. In some embodiments, the concentration of PEG400 (or any other buffer additives such as bovine serum albumin (BSA), polyethylene glycol or other additive concentrations, antioxidants and reducing agents, etc.) may range from about 0.01% (w/v) to about 10% (w/v). In some embodiments, the concentration of PEG400 (or any other buffer additive) may be at least 0.01%, at least 0.05%, at least 0.1%, at least 0.5%, at least 1.0%, at least 1.5%, at least 2%, at least 3%, at least 4%, at least 5%, at least 6%, at least 7%, at least 8%, at least 9%, or at least 10%. In some embodiments, the concentration of PEG400 (or any other buffer additive) may be at most 10%, at most 9%, at most 8%, at most 7%, at most 6%, at most 5%, at most 4%, at most 3%, at most 2%, at most 1.5%, at most 1.0%, at most 0.5%, at most 0.1%, at most 0.05%, or at most 0.01%. Any of the lower and upper values described in this paragraph may be combined to form a range included within the present disclosure, for example, the concentration of PEG400 (or any other buffer additive) may range from about 0.5% (w/v) to about 5% (w/v). Those of skill in the art will recognize that the concentration of PEG400 (or any other buffer additive) may have any value within this range, for example, about 2.25% (w/v).

In some embodiments, the number of different sets of experimental conditions used for SHG and/or TPF polarization measurements may be increased in order to increase the number of independent molecular orientational distributions to be sampled and the number of independent polarization measurements that may be made, thereby increasing the accuracy of the angular measurements and the protein structural models derived therefrom. In some embodiments, the number of different sets of experimental conditions used may be at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, or at least 100.

Nonlinear-active labels and labeling techniques: As noted above, most biological molecules are not intrinsically TPF-active or SH-active. Exceptions include collagen, a structural protein that is found in most structural or load-bearing tissues. SHG microscopy has been used extensively in studies of collagen-containing structures, for example, the cornea. Other biological molecules or entities must be rendered nonlinear-active by means of introducing a nonlinear-active moiety such as a tag or label. A label for use in the present invention refers to a nonlinear-active moiety, tag, molecule, or particle which can be bound, either covalently or non-covalently to a molecule, particle, or phase (e.g., a lipid bilayer) in order to render the resulting system more nonlinear optical active. Labels can be employed in the case where the molecule, particle, or phase (e.g., a lipid bilayer) is not nonlinear active to render the system nonlinear-active, or with a system that is already nonlinear-active to add an extra characterization parameter into the system. Exogenous labels can be pre-attached to the molecules, particles, or other biological entities, and any unbound or unreacted labels separated from the labeled entities before use in the methods described herein. In a specific aspect of the methods disclosed herein, the nonlinear-active moiety is attached to the target molecule or biological entity in vitro prior to immobilizing the target molecules or biological entities in discrete regions of the substrate surface. In another aspect of the methods disclosed herein, the nonlinear-active moiety is attached to the target molecule or biological entity after immobilizing the target molecules or biological entities in discrete regions of the substrate surface. The labeling of biological molecules or other biological entities with nonlinear-active labels allows a direct optical means of detecting interactions between the labeled biological molecule or entity and another molecule or entity (i.e. the test entity) in cases where the interaction results in a change in orientation or conformation of the biological molecule or entity using a surface-selective nonlinear optical technique.

Examples of nonlinear-active tags or labels suitable for use in the disclosed methods include, but are not limited to, the compounds listed in Table 3, and their derivatives.

TABLE 3
Examples of Nonlinear-Active Tags

| | | |
|---|---|---|
| 2-aryl-5-(4-pyridyl)oxazole | Hemicyanines | Polyimides |
| 2-( 4-pyridy1)- | lndandione-1,3-pyidinium betaine | Polymethacrylates |
| cycloalkano[d]oxazoles | | |
| 5-aryl-2-(4-pyridyl)oxazole | lndodicarbocyanines | PyMPO (pyridyloxazole) |
| 7-Hydroxycoumarin-3-carboxylic | Melamines | PyMPO, SE, 1-(3- |
| acid, succinimidyl ester | | (Succinimidyloxycarbonyl)Benzyl)- |
| | | 4-(5-(4-Methoxyphenyl)Oxazol- |
| | | 2-y1)Pyridinium Bromide |
| | | PyMPO maleimide, 1-(2- |
| | | maleimidylethyl)-4-(5-(4- |
| | | methoxyphenyl) oxazol-2- |
| | | yl)pyridinium methanesulfonate |
| Azo dyes | Merocyanines | Stilbazims |
| Benzooxazoles | Methoxyphenyl)oxazol-2- | Stilbenes |
| | yl)pyridinium bromide) | |
| Bithiophenes | Methylene blue | Stryryl-based dyes |
| Cyanines | Oxazole or oxadizole molecules | Sulphonyl-substituted azobenzenes |
| Dapoxyl carboxylic acid, | Oxonols | Thiophenes |
| succinimidyl ester | | |
| Diaminobenzene compounds | Perylenes | Tricyanovinyl aniline |
| Diazostilbenes | Phenothiazine-stilbazole | Tricyanovinyl azo |
| Fluoresceins | Polyenes | |

In evaluating whether a species may be nonlinear-active, the following characteristics can indicate the potential for nonlinear activity: a large difference dipole moment (difference in dipole moment between the ground and excited states of the molecule), a large Stokes shift in fluorescence, or an aromatic or conjugated bonding character. In further evaluating such a species, an experimenter can use a simple technique known to those skilled in the art to confirm the nonlinear activity, for example, through detection of SHG from an air-water interface on which the nonlinear-active species has been distributed.

Once a suitable nonlinear-active species has been selected for the experiment at hand, the species can be conjugated, if desired, to a biological molecule or entity for use in the surface-selective nonlinear optical methods and systems disclosed herein. The following reference and references cited therein describe techniques available for creating a labeled biological entity from a synthetic dye and many other molecules: Greg T. Hermanson, Bioconjugate Techniques, Academic Press, New York, 1996. In general, an important consideration for performing labeling reactions is the specificity and yield of the reaction, which should be maximized to ensure that consistent, reproducible baseline SHG signals (or related nonlinear optical signals) are achieved.

In some embodiments, the attachment of nonlinear-active labels to protein molecules may be performed using standard covalent conjugation chemistries, e.g. using non-linear active moieties that are reactive with amine groups, carboxyl groups, thiol groups, and the like. In some embodiments, it may optionally be desirable to perform mass spectrometric analysis of the labeled proteins to rigorously identify the positions of the labeled amino acid residues within the protein.

Examples of suitable amine-reactive conjugation chemistries that may be used include, but are not limited to, reactions involving isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester groups. Examples of suitable carboxyl-reactive conjugation chemistries include, but are not limited to, reactions involving carbodiimide compounds, e.g., water soluble EDC (1-ethyl-3-(3-dimethylaminopropyl)carbodiimide.HCL). Examples of suitable sulfydryl-reactive conjugation chemistries include maleimides, haloacetyls and pyridyl disulfides.

In some embodiments, the nonlinear-active label is a two-photon fluorescent and/or second harmonic (SH)-active label selected from the group consisting of pyridyloxazole (PyMPO), PyMPO maleimide, PyMPO-NHS, PyMPO-succinimidyl ester (PyMPO-SE), 6-bromoacetyl-2-dimethylaminonaphthalene (Badan), and 6-Acryloyl-2-dimethylaminonaphthalene (Acrylodan). In order to achieve a high degree of assay reproducibility (e.g., as indicated by tight coefficients of variation (CVs) for replicate measurements using the same protein sample), the number of labeling steps (and tethering or immobilization steps) required to prepare the protein sample for TPG or SHG signal measurements should be minimized. For example, ideally, the labeling reaction should be highly specific for the label attachment site on the protein, should be very high yield (i.e., should yield 1:1 stoichiometric labeling), and should not require a post-labeling separation step. PyMPO maleimide is a suitable nonlinear-active label which is both SHG- and TPF-active. A PyMPO analog equipped with methionine-chemoselective chemistry is also suitable. Methionine chemoselective chemistry is described in Lin, et al. (2017), “Redox-Based Reagents for Chemoselective Methionine Bioconjugation”, Science 355(6325):597-602.

In some embodiments, the nonlinear-active label is bound to the protein by one or more sulfhydryl groups, amine groups, or carboxyl groups on the surface of the protein. In some embodiments, the one or more sulfhydryl groups, amine groups, or carboxyl groups are native sulfhydryl groups, amine groups, or carboxyl groups. In some embodiments, the one or more sulfhydryl groups, amine groups, or carboxyl groups are engineered sulfhydryl groups, amine groups, or carboxyl groups. Genetic engineering and site-directed mutagenesis techniques for engineering the attachment sites for the labels are well known to those of skill in the art (see, for example, Edelheit, et al. (2009), “Simple and Efficient Site-Directed Mutagenesis Using Two Single-Primer Reactions in Parallel to Generate Mutants for Protein Structure-Function Studies”, BMC Biotechnology 9:61). Using this approach, amino acid residues (e.g., cysteine, lysine, aspartate, or glutamate residues) comprising sulfhydryl (i.e., thiol) groups, amine groups, or carboxyl groups, for example, may be placed at precise positions within the protein prior to labeling with a nonlinear-active tag. In a preferred embodiment, the engineered labeling sites comprise substitution of cysteine residues for native amino acid residues. Often, the labeling site for which an amino acid residue is substituted may be an amino acid residue at a position in the protein's amino acid sequence that is known to be located on the surface of the protein when the protein is properly folded. Mutated and labeled mutated proteins may then be tested for native-like functionality using any of a variety of assays known to those of skill in the art, e.g., by performing binding assays using a known ligand for the protein. In some embodiments, a series of mutant proteins may be prepared, wherein each mutant comprises a nonlinear-active tag attached at a different site within the protein molecule. In some embodiments, a mutant protein may comprise a single amino acid substitution that is used for labeling. In some embodiments, a mutant protein may comprise two or more amino acid substitutions that are used for labeling. In some embodiments, mutant proteins may further comprise an amino acid substitution (e.g., a lysine, cysteine, methionine, aspartate, or glutamate residue) in addition to the engineered labeling site(s) that is used for tethering the labeled protein to a substrate surface or supported lipid bilayer by means of a suitable linker molecule, as will be described in more detail below. In some embodiments, the nonlinear-active label is a second harmonic generation (SHG)-active label, a sum frequency generation (SFG)-active label, a difference frequency (DFG)-active label, or a two-photon fluorescence (TPF)-active label. In some embodiments, the nonlinear-active label is both SHG-active and TPF-active.

In other preferred embodiments, genetic engineering techniques may be used to incorporate nonlinear-active unnatural amino acids at specific sites within the protein using any of a variety of in vivo or cell-free in vitro techniques known to those of skill in the art. See, for example, Cohen, et al. (2002), “Probing Protein Electrostatics with a Synthetic Fluorescence Amino Acid”, Science 296:1700-1703, and U.S. Pat. No. 9,182,406. In some embodiments, nonlinear-active unnatural amino acid residues may be incorporated into a family of mutant proteins comprising nonlinear-active unnatural amino acid substitutions at one, two, three, four, or five or more known sites. Such proteins can be engineered, naturally occurring, made using in vitro translation methods, expressed in vivo, and in general created through any of the various methods known to those skilled in the art. In some embodiments, the nonlinear-active unnatural amino acid is L-Anap, Aladan, or another derivative of naphthalene. In some embodiments, the incorporation of an intrinsically nonlinear-active unnatural amino acid residue into a biological drug candidate may be tested for any deleterious effects on the structure or function of the drug candidate compared to a reference drug, and if there are none, may subsequently be used as an internal marker for quality control during manufacturing of the biosimilar drug.

In a specific aspect of the methods and systems disclosed, metal nanoparticles and assemblies thereof are modified to create biological nonlinear-active labels. The following references describe the modification of metal nanoparticles and assemblies: J. P. Novak and D. L. Feldheim, “Assembly of Phenylacetylene-Bridged Silver and Gold Nanoparticle Arrays”, J. Am. Chem. Soc. 122:3979-3980 (2000); J. P. Novak, et al., “Nonlinear Optical Properties of Molecularly Bridged Gold Nanoparticle Arrays”, J. Am. Chem. Soc. 122:12029-12030 (2000); Vance, F. W., Lemon, B. I., and Hupp, J. T., “Enormous Hyper-Rayleigh Scattering from Nanocrystalline Gold Particle Suspensions”, J. Phys. Chem. B 102:10091-93 (1999).

In some embodiments, target proteins (e.g., drug target proteins, biological drug candidates, biological reference drugs, drug target proteins, etc.) may be rendered nonlinear-active through binding of nonlinear-active peptides which bind specifically and/or reversibly to the protein molecules through local non-covalent forces such as electrostatic interactions, hydrogen bonding, hydrophobic interactions and/or van der Waals interactions, or any combination thereof. One or more peptides labeled with an SHG-active, SFG-active, DFG-active, and/or TPF-active moiety may be synthesized and reacted with the protein of interest (before or after tethering of the target protein to the optical interface) and tested for their ability to bind to the target protein in a specific manner (e.g., using SHG and/or TPF measurements to determine the width of the distribution of orientational angles for the nonlinear-active moiety on the bound peptide, where the orientational distribution of the tethered target protein molecules is independently known from SHG and/or TPF measurements made for tethered target protein molecules that have been directly labeled with a nonlinear active label either through covalent conjugation or through genetic incorporation of a nonlinear-active unnatural amino acid). In a preferred embodiment, the nonlinear-active peptide may comprise a peptide sequence known to bind to a specific protein domain. For example, octamer peptide sequences (e.g., NKFRGKYK and NARKFYKG) that bind specifically to the Fc portion of mouse or human IgG molecules with high affinity (8.9×10⁶ M⁻¹ and 6.5×10⁶ M⁻¹ respectively for human IgG-Fc) have been reported in the literature (Sugita, et al. (2013), Biochem. Eng. J. 79:33-40).

One very useful embodiment is to label a peptide, peptidomimetic or small molecule probe, for example, one known to bind to the complimentarity-determining region (CDR) in an antibody, perhaps a fragment of the antigen. Such a peptide, for example, can be made in solid phase synthesis with a non-native amino acid that is SHG- and/or TPF-active (e.g., L-Anap), or a version of PyMPO that is an amino acid; or such a peptide can be labeled in solution using PyMPO-NHS or PyMPO-maleimide, for example, according to methods known to those skilled in the art. These labeled peptides can then be bound to antibody which itself is tethered to a surface, for example a supported lipid bilayer membrane comprising Ni-NTA bearing lipids. The SHG-active peptide can then be contacted with the antibody to produce a baseline signal. The antibody can be “stressed” by heat, light, or other means, for example, and the baseline signal of this stressed sample can be compared to that of the unstressed sample. Similarly, baseline signals can be compared in a bioprocess monitoring setting to ensure that a biologic is maintaining a constant structure at the region probed by the labeled peptide. Furthermore, generic vs. brand biologics can be compared in a similar manner. In such cases (and/or in other various instances), one may rely on the high sensitivity of SHG and/or TPF to orientation and/or structure, and the ability to label a target protein or molecule of interest such as a biologic or antibody using a peptide or some other ligand that itself has been rendered or made SHG- and/or TPF-active.

In yet another aspect of the methods and systems disclosed herein, the nonlinear activity of the system can also be manipulated through the introduction of nonlinear analogues to molecular beacons, that is, molecular beacon probes that have been modified to incorporate a nonlinear-active label (or modulator thereof) instead of fluorophores and quenchers. These nonlinear optical analogues of molecular beacons are referred to herein as molecular beacon analogues (MB analogues or MBA). The MB analogues to be used in the described methods and systems can be synthesized according to procedures known to one of ordinary skill in the art.

In some embodiments, one or more nonlinear-active labels may be attached to one or more known positions (e.g., sites) within the same individual biomolecule, e.g., a protein molecule. In some embodiments, the one or more nonlinear-active labels may be attached to one or more known positions (e.g. sites) in different molecules of the same protein, i.e. to create a family of proteins comprising different single-label versions of the labeled protein. In some embodiments, the number of labeling sites at which the protein (or family of proteins) is labeled may be at least 1 site, at least 2 sites, at least 3 sites, at least 4 sites, at least 5 sites, at least 6 sites, at least 7 sites, at least 8 sites, at least 9 sites, at least 10 sites, or more. In some embodiments, the nonlinear-active label may be attached to different single-site cysteine mutants or variants of the same protein. In some embodiments, the nonlinear-active labels located at the one, two, three, or more known positions are the same. In some embodiments, the nonlinear-active labels located at the one, two, three, or more known positions are different. In some embodiments, the one or more nonlinear-active labels are two-photon active labels. In some embodiments, the one or more nonlinear-active labels are two-photon active and/or one or more of the following: second harmonic (SH)-active, sum-frequency (SF)-active, or difference frequency (DF)-active.

In some embodiments, SHG and/or TPF measurements may comprise using protein molecules labeled with a single nonlinear-active label. In some embodiments, the SHG and/or TPF measurements may comprise using protein molecules labeled with at least 2 different nonlinear-active labels, at least 3 different nonlinear-active labels, at least 4 different nonlinear-active labels, at least 5 different nonlinear-active labels, at least 6 different nonlinear-active labels, at least 7 different nonlinear-active labels, at least 8 different nonlinear-active labels, at least 9 different nonlinear-active labels, or at least 10 different nonlinear-active labels.

In alternative aspects of the methods and systems described herein, at least two distinguishable nonlinear-active labels are used. The orientation of the attached two or more distinguishable labels would then be chosen to facilitate well defined directions of the emanating coherent nonlinear light beam. The two or more distinguishable labels can be used in assays where multiple fundamental light beams at one or more frequencies, incident with one or more polarization directions relative to the optical interface are used, with the resulting emanation of at least two nonlinear light beams. In some embodiments, the number of distinguishable nonlinear-active labels used may be at least two, at least three, at least four, at least five, at least six, at least seven, at least eight, at least nine, or at least ten.

In some embodiments, the at least two distinguishable nonlinear-active labels may be used in combination with multiple fundamental light beams at one or more frequencies, incident with one or more polarization directions relative to the optical interface, to determine relative or absolute tilt angles for each of the at least two distinguishable nonlinear-active labels relative to each other or relative to the surface normal for the optical interface, thereby facilitating the mapping of protein structure (provided that the labeling sites for each distinguishable nonlinear-active label within the protein is known), or to facilitate the mapping of local conformational changes upon binding of a ligand to the protein.

Tethering and immobilization chemistries: As disclosed herein, substrates in any of the formats described below are configured for tethering of proteins or other biomolecules (or in some cases, cells or other biological entities) on all or a portion of the substrate. In many embodiments, the substrate may be configured for tethering or immobilization of proteins or other biomolecules within specified discrete regions of the substrate. Tethering (sometimes referred to herein as “attachment” or “immobilization”) of biological molecules or cells may be accomplished by a variety of techniques known to those of skill in the art, for example, through the use of aminopropyl silane chemistries to functionalize glass or fused-silica surfaces with amine functional groups, followed by covalent coupling using amine-reactive conjugation chemistries, either directly with the biological molecule of interest, or via an intermediate spacer or linker molecule. Non-specific adsorption may also be used directly or indirectly, e.g. through the use of BSA-NHS (BSA-N-hydroxysuccinimide) by first attaching a molecular layer of BSA to the surface and then activating it with N,N′-disuccinimidyl carbonate. The activated lysine, aspartate or glutamate residues on the BSA react with surface amines on proteins.

Use of supported lipid bilayers: In a preferred aspect of the present disclosure, biological molecules may be tethered to the surface by means of tethering to or embedding in “supported lipid bilayers”, the latter comprising small patches of lipid bilayer confined to a silicon or glass surface by means of hydrophobic and electrostatic interactions, where the bilayer is “floating” above the substrate surface on a thin layer of aqueous buffer. Supported phospholipid bilayers can also be prepared with or without membrane proteins or other membrane-associated components as described, for example, in Salafsky et al., “Architecture and Function of Membrane Proteins in Planar Supported Bilayers: A Study with Photosynthetic Reaction Centers”, Biochemistry 35 (47): 14773-14781 (1996); Gennis, R., Biomembranes, Springer-Verlag, 1989; Kalb et al., “Formation of Supported Planar Bilayers by Fusion of Vesicles to Supported Phospholipid Monolayers”, Biochimica Biophysica Acta. 1103:307-316 (1992); and Brian et al. “Allogeneic Stimulation of Cytotoxic T-cells by Supported Planar Membranes”, PNAS-Biological Sciences 81(19): 6159-6163 (1984), relevant portions of which are incorporated herein by reference. Supported phospholipid bilayers are well known in the art and there are numerous techniques available for their fabrication. Potential advantages of using supported lipid bilayers for immobilization of proteins or other biological entities on substrate surfaces or optical interfaces include (i) preservation of membrane protein structure for those proteins that typically span the cell membrane or other membrane components of cells and require interaction with the hydrophobic core of the bilayer for stabilization of secondary and tertiary structure, (ii) preservation of two dimensional lateral and rotational diffusional mobility for studying interactions between protein components within the bilayer, and (iii) preservation of molecular orientation, depending on such factors as the type of protein under study (i.e. membrane or soluble protein), how the bilayer membrane is formed on the substrate surface, and how the protein is tethered to the bilayer (in the case of soluble proteins). Supported bilayers, with or without tethered or embedded protein, should typically be submerged in aqueous solution to prevent their destruction when exposed to air.

In some embodiments of the disclosed methods and devices, it may be advantageous to vary the lipid composition of the supported lipid bilayer, e.g., the number of different lipid components and/or their relative concentrations, in order to improve binding of protein molecules (e.g., peripheral membrane proteins), preserve the native structure of membrane or peripheral membrane proteins, and/or to mimic the physiological responses observed in vivo. Examples of lipid molecules that may be used to form supported lipid bilayers or that may be inserted as major or minor components of the supported lipid bilayer include, but are not limited to, diacylglycerol, phosphatidic acid (PA), phosphatidylethanolamine (PE), phosphatidylcholine (PC), phosphatidylserine (PS), phosphatidylinositol (PI), phosphatidylinositol phosphate (PIP), phosphatidylinositol biphosphate (PIP2), phosphatidylinositol triphosphate (PIP3), ceramide phosphorylcholine (sphingomyelin; SPH), ceramide phosphorylethanolamine (sphingomyelin; Cer-PE), ceramide phosphoryllipid, cholesterol, or any combination thereof.

In some embodiments the number of different lipid components of the supported lipid bilayer may range from 1 to 10, or more. In some embodiments, the number of different lipid components may be at least 1, at least 2, at least 3, at least 4, at least 5, at least 6, at least 7, at least 8, at least 9, or at least 10. In some embodiments, the number of different lipid components may be at most 10, at most 9, at most 8, at most 7, at most 6, at most 5, at most 4, at most 3, at most 2, or at most 1.

In some embodiments, the relative percentage of a given lipid component of the supported lipid bilayer may range from about 0.1% to about 100%. In some embodiments, the relative percentage of a given lipid component may be at least about 0.1%, at least about 0.2%, at least about 0.3%, at least about 0.4%, at least about 0.5%, at least about 1%, at least about 5%, at least about 10%, at least about 20%, at least about 30%, at least about 40%, at least about 50%, at least about 60%, at least about 70%, at least about 80%, at least about 90%, or at least about 100%. In some embodiments, the relative percentage of a given lipid component may be at most about 100%, at most about 90%, at most about 80%, at most about 70%, at most about 60%, at most about 50%, at most about 40%, at most about 30%, at most about 20%, at most about 10%, at most about 5%, at most about 1%, at most about 0.5%, at most about 0.4%, at most about 0.3%, at most about 0.2%, or at most about 0.1%. Those of skill in the art will recognize that the relative percentage of a given lipid component in the supported lipid bilayer may have any value within this range, e.g., about 12.5%.

In those embodiments where the supported lipid bilayer comprises two or more different lipid components, the relative percentages of the two or more different lipid components may be the same or may be different. In one non-limiting example, the supported lipid bilayer may comprise 25% PS, 74.5% PC and 0.5% Lissamine Rhodamine PE. In another non-limiting example, the supported lipid bilayer may comprise 5% PIP, 20% PS, 74.5% PC and 0.5% Lissamine Rhodamine PE. For some types of lipid, the requirements for forming a stable supported lipid bilayer may limit the relative percentage of that lipid in the bilayer to less than 100%. In these cases, the relative percentage of the de-stabilizing lipid component may typically range from about 1% to about 50%. In some embodiments, the relative percentage of the de-stabilizing lipid component in the supported lipid bilayer may be at least about 1%, at least about 5%, at least about 10%, at least about 20%, at least about 30%, at least about 40%, or at least about 50%. In some embodiments, the relative percentage of the de-stabilizing lipid component in the supported lipid bilayer may be at most about 50%, at most about 40%, at most about 30%, at most about 20%, at most about 10%, at most about 5%, or at most about 1%. Those of skill in the art will recognize that the relative percentage of a de-stabilizing lipid component in the supported lipid bilayer may have any value within this range, e.g., about 12.5%.

As indicated above, the supported lipid bilayer may also comprise target proteins, or subunits, subdomains, or fragments thereof. In some embodiments, the supported lipid bilayer may also include non-integral protein components that are tethered to the lipid bilayer, e.g., through covalent or non-covalent coupling to a lipid-like or hydrophobic moiety that inserts itself into the lipid bilayer.

In some embodiments, the number of different protein components (integral or non-integral) included in the supported lipid bilayer may range from about 1 to about 10 or more. In some embodiments, the number of different protein components may be at least 1, at least 2, at least 3, at least 4, at least 5, at least 6, at least 7, at least 8, at least 9, or at least 10. In some embodiments, the number of different protein components may be at most 10, at most 9, at most 8, at most 7, at most 6, at most 5, at most 4, at most 3, at most 2, or at most 1.

In some embodiments, the molar fraction of a given protein component of the lipid bilayer may range from about 0.1 to about 1. In some embodiments, the molar fraction of a given protein component may be at least about 0.1, at least about 0.2, at least about 0.3, at least about 0.4, at least about 0.5, at least about 0.6, at least about 0.7, at least about 0.8, at least about 0.9, or at least about 1. In some embodiments, the molar fraction of a given protein component may be at most about 1, at most about 0.9, at most about 0.8, at most about 0.7, at most about 0.6, at most about 0.5, at most about 0.4, at most about 0.3, at most about 0.2, or at most about 0.1. Those of skill in the art will recognize that the molar fraction of a given protein component in the lipid bilayer may have any value within this range, e.g., about 0.15%.

Anchor molecules, linkers, and attachment chemistries: Soluble proteins and other biological entities may be tethered or attached to the supported lipid bilayer (or directly to the substrate that comprises the optical interface) in an oriented fashion using a number of different anchor molecules, linkers, and/or attachment chemistries. As used herein, “anchor molecules” are molecules which are embedded in the lipid bilayer, and may comprise fatty acid, glycerolipid, glycerophospholipid, sphingolipid, or other lipid or non-lipid molecules to which attachment moieties are conjugated.

Linker molecules are molecules used to provide spatial (“vertical”) separation between the attachment point of the protein or other biological entity being tethered and the attachment point on the anchor molecule embedded in the plane of the lipid bilayer. In some embodiments, linker molecules may be used to provide spatial separation between the attachment point of the protein or other biological entity being tethered and an attachment point directly on the substrate that comprises the optical interface. Examples of suitable linker molecules include, but are not limited to, omega-amino fatty acids, polyethylene glycols, and the like.

Attachment moieties (also referred to as “affinity tags”) are specific chemical structures or binding partners that provide for covalent or non-covalent binding between two biological entities. Examples of attachment moieties or affinity tags that are suitable for use in the methods disclosed herein include biotin and avidin (or biotin and streptavidin), and His-tag/Ni-NTA binding partners.

The high affinity, non-covalent biotin-streptavidin interaction is widely used in biological assay techniques to conjugate or immobilize proteins or other biological entities. Biotinylation of proteins enables capture by multivalent avidin or streptavidin molecules that are themselves adhered to a surface (e.g. glass slides or beads) or conjugated to another molecule (e.g. through the use of a biotin-streptavidin-biotin bridge or linker). The biotin moiety is sufficiently small that biotinylation typically doesn't interfere with protein function. The high affinity (Kd of 10⁻¹⁴ M to 10⁻¹⁵M) and high specificity of the binding interaction between biotin and avidin or streptavidin enables capture of biotinylated proteins of interest even from complex samples. Due to the extremely strong binding interaction, harsh conditions are needed to elute biotinylated protein from streptavidin-coated surfaces (typically 6M guanidine HCl at pH 1.5), which will often denature the protein of interest. The use of monomeric forms of avidin or streptavidin, which have a decreased biotin-binding affinity of about10⁻⁸ M, may allow biotinylated proteins to be eluted with excess free biotin if necessary. In the methods disclosed herein, lipid molecules comprising biotin moieties may be incorporated into supported lipid bilayers for the purpose of immobilizing or tethering biotinylated proteins and/or other biotinylated biological entities to the bilayer via a biotin-avidin-biotin (or biotin-streptavidin-biotin) bridge.

Biotinylation of proteins and other biological entities may be performed by direct coupling, e.g. through conjugation of primary amines on the surface of a protein using N-hydroxysuccinimidobiotin (NHS-biotin). Alternatively, recombinant proteins are conveniently biotinylated using the AviTag approach, wherein the AviTag peptide sequence (GLNDIFEAQKIEWHE) is incorporated into the protein through the use of genetic engineering and protein expression techniques. The presence of the AviTag sequence allows biotinylation of the protein by treatment with the BirA enzyme.

His tag chemistry is another widely used tool for purification of recombinant proteins and other biomolecules. In this approach, for example, a DNA sequence specifying a string of six to nine histidine residues may be incorporated into vectors used for production of recombinant proteins comprising 6× His or poly-His tags fused to their N- or C-termini. In some embodiments, target proteins or other biological proteins may be engineered to include, e.g., a 2×-His tag, a 3×-His tag, a 4×-His tag, a 5×-His tag, a 6×-His tag, a 7×-His tag, an 8×-His tag, a 9×-His tag, a 10×-His tag, an 11×-His tag, or a 12×-His tag, that binds to a bilayer lipid comprising a Ni-NTA moiety.

His-tagged proteins can then be purified and detected as a result of the fact that the string of histidine residues binds to several types of immobilized metal ions, including nickel, cobalt and copper, under specific buffer conditions. Supports such as agarose beads or magnetic particles can be derivatized with chelating groups to immobilize the desired metal ions, which then function as ligands for binding and purification of the His-tagged biomolecules of interest.

The chelators most commonly used to create His-tag ligands are nitrilotriacetic acid (NTA) and iminodiacetic acid (IDA). Once NTA- or IDA-conjugated supports are prepared, they can be “loaded” with the desired divalent metal (e.g., Ni, Co, Cu, or Fe). When using nickel as the metal, for example, the resulting affinity support is usually called a Ni-chelate, Ni-IDA or Ni-NTA support. Affinity purification of His-tagged fusion proteins is the most common application for metal-chelate supports in protein biology research. Nickel or cobalt metals immobilized by NTA-chelation chemistry are the systems of choice for this application. In the methods disclosed herein, lipid molecules comprising Ni-NTA groups (or other chelated metal ions) may be incorporated into supported lipid bilayers for the purpose of immobilizing or tethering His-tagged proteins and other His-tagged biological entities to the bilayer. In some embodiment, the supported lipid bilayer may comprise 1,2-dioleoyl-sn-glycero-3-phosphocholine, and may also contain 1,2-dioleoyl-sn-glycero-3-[(N-(5-amino-1-carboxypentyl)iminodiacetic acid)succinyl] (nickel salt) at various concentrations.

Poly-His tags bind best to chelated metal ions in near-neutral buffer conditions (physiologic pH and ionic strength). A typical binding/wash buffer consists of Tris-buffer saline (TBS) pH 7.2, containing 10-25 mM imidazole. The low-concentration of imidazole helps to prevent nonspecific binding of endogenous proteins that have histidine clusters. Elution and recovery of captured His-tagged protein from chelated metal ion supports, when desired, is typically accomplished using a high concentration of imidazole (at least 200 mM), low pH (e.g., 0.1M glycine-HCl, pH 2.5), or an excess of strong chelator (e.g., EDTA). Immunoglobulins are known to have multiple histidines in their Fc region and can bind to chelated metal ion supports, therefore stringent binding conditions (e.g. using an appropriate concentration of imidazole) are necessary to avoid high levels of background binding if immunoglobulins are present in a sample at high relative abundance compared to the His-tagged proteins of interest. Albumins, such as bovine serum albumin (BSA), also have multiple histidines and can yield high levels of background binding to chelated metal ion supports in the absence of more abundant His-tagged proteins or the use of imidazole in the binding/wash buffer.

In some embodiments, substrate surfaces derivatized with Ni/NTA, or other metal ion chelators, may be used to immobilize proteins that lack a His-tag. For example, monoclonal antibodies (mAbs) will associate quite readily with a Ni/NTA surface upon contact with the surface. See Block, et al. (2009), “Immobilized-Metal Affinity Chromatography (IMAC): A Review”, in Methods in Enzymology, Volume 463, Chapter 27, Elsevier, for additional examples of immobilization of proteins on surfaces derivatized with metal ion chelates.

In some embodiments, a target protein (e.g., drug target proteins, biological drug candidates, biological reference drugs, drug target proteins, etc.) may be a protein that has been genetically-engineered to incorporate a unique tethering or immobilization site for attaching the protein to the optical interface, and/or one which has been genetically-engineered to incorporate an unnatural amino acid residue that serves as a unique tethering or immobilization site for attachment of the protein to the optical interface. Examples of unique tethering or immobilization sites that may be genetically-incorporated include, but are not limited to, incorporation of a lysine, aspartate, or glutamate residue at an amino acid sequence position that is known to be located on the surface of the protein when the protein is properly folded. The protein may then be tethered to or immobilized on the optical interface using any of a variety of conjugation and linker chemistries known to those of skill in the art. Another non-limiting example of a unique tethering or immobilization site that may be genetically-incorporated into a protein product may be a His tag (i.e., a series of 1, 2, 3, 4, 5, 6, 7, 8, 9, 10, 11, 12, or more than 12 histidine residues that may then provide an attachment site for binding to Ni/NTA groups attached to the optical interface. One non-limiting example of an unnatural amino acid that may be incorporated to provide a unique attachment point is the biotinylated unnatural amino acid biocytin. The protein may then be tethered to or immobilized on the optical interface using the high-affinity biotin-streptavidin interaction to tether the protein to streptavidin molecules immobilized on the substrate surface.

Rinsing/washing after tethering: In some embodiments, it may be advantageous to avoid rinsing or washing the substrate surface after the protein tethering or immobilization step, i.e., any residual protein that has not been tethered or immobilized may be left in the well, reaction chamber, or compartment used to bring the protein sample into contact with the substrate surface. Because of the surface-selective nature of the disclosed measurement techniques, and their dependence on net orientation of the protein molecules at the optical interface, any residual labeled protein in solution will provide little or no contribution to the measured nonlinear optical signal.

Saturation of binding sites on substrate surfaces: In general, the concentration of protein in the one or more samples to be analyzed should be sufficiently high to ensure saturation of the binding sites on the substrate surface under the set of incubation conditions used for tethering or immobilization. This is to maximize consistency in preparation of the sample for baseline nonlinear optical signal measurements. In some embodiments, the concentration of the protein in the one or more samples to be analyzed is the same, and may be the same as that in a reference sample. In some embodiments, e.g., if the protein concentration in the sample aliquot is low, it may be desirable to provide substrates with lower binding site density and/or to use longer incubation times to ensure saturation of binding sites.

Varying the surface density of tethered molecules: In those embodiments where it is desirable to vary the surface density of protein binding sites on the substrate surface, control of surface binding site density may be accomplished in a variety of ways known to those of skill in the art. For example, in embodiments where proteins are coupled to the surface through the use of aminopropyl silane chemistries to functionalize glass or fused-silica surfaces with amine functional groups, followed by covalent coupling using amine-reactive conjugation chemistries and linker molecules, the ratio of bi-functional linker molecules (e.g., linkers comprising both a primary amine and carboxyl functional group) to mono-functional linker molecules (e.g., comprising only a carboxyl functional group) in the reaction mixture may be varied to control the surface density of primary amine functional groups available for coupling with the protein. The surface density of tethered (labeled) molecules can also be varied by simply incubating at different concentrations of the molecules in solution.

As another example, in embodiments where biotin-streptavidin binding interactions are used to tether biotinylated proteins to biotinylated lipid molecules incorporated into a supported lipid bilayer (via a biotin-streptavidin-biotin bridge), the mole percent of the biotinylated lipid molecule used to form the bilayer may be varied in order to control the surface density of biotin groups available for binding.

As yet another example, in embodiments where His-tagged proteins are immobilized to supported lipid bilayers using anchor lipid molecules comprising Ni-NTA (or other chelated metal ion) ligands, the mole percent of Ni-NTA-containing lipid molecule used to form the bilayer may be varied in order to control the surface density of Ni-NTA ligands available for binding.

In some embodiments, the density of attachment sites on the supported lipid bilayer may be varied by varying the percentage of a lipid component of the bilayer that comprises an amine group or a thiol group (or any other functional group for which standard conjugation chemistries are available). In some embodiments, the percentage of the lipid component that comprises an amine or thiol group may range from about 0 percent to about 100 percent. In some embodiments, the percentage of the lipid component that comprises an amine or thiol group may be at least 0 percent, at least 10 percent, at least 20 percent, at least 30 percent, at least 40 percent, at least 50 percent, at least 60 percent, at least 70 percent, at least 80 percent, at least 90 percent, or at least 100 percent. In some embodiments, the percentage of the lipid component that comprises an amine or thiol group may be at most 100 percent, at most 90 percent, at most 80 percent, at most 70 percent, at most 60 percent, at most 50 percent, at most 40 percent, at most 30 percent, at most 20 percent, or at most 10 percent. Those of skill in the art will recognize that the percentage of the lipid component that comprises an amine group or a thiol group may have any value within this range, for example, about 12 percent.

In some embodiments, the density of nonlinear-active labeled proteins attached to the surface may be varied by varying the concentration of labeled protein in the solution that is incubated with the supported lipid bilayer. For example, the concentration of a His tagged, labeled protein may be varied in the solution that is incubated with a supported lipid bilayer comprising a lipid component that further comprises a Ni-NTA moiety. In some embodiment, the concentration of labeled protein in the solution may range from about 1 nM to about 100 μM. In preferred embodiments, the concentration of labeled protein in the solution may range from about 100 nM to about 5 μM. In some embodiments, the concentration of labeled protein in the solution may be at least 1 nM, at least 10 nM, at least 100 nM, at least 1 μM, at least 10 μM, or at least 100 μM. In some embodiments, the concentration of labeled protein in the solution may be at most 100 μM, at most 10 μM, at most 1 μM, at most 100 nM, at most 10 nM, or at most 1 nM. Those of skill in the art will recognize that the concentration of labeled protein in the solution may have any value with this range, for example, about 12 μM.

In some embodiments the density of nonlinear-active labeled protein on the surface may be varied using any of a variety of techniques known to those of skill in the art over the range of about 10² molecules/cm² to about 10¹⁴ molecules/cm². In some embodiments, the density of nonlinear-active labeled protein on the surface may be at least 10² molecules/cm², at least 10³ molecules/cm², at least 10⁴ molecules/cm², at least 10⁵ molecules/cm², at least 10⁶ molecules/cm², at least 10⁷ molecules/cm², at least 10⁸ molecules/cm², at least 10⁹ molecules/cm², at least 10¹⁰ molecules/cm², at least 10¹¹ molecules/cm², at least 10¹² molecules/cm², at least 10¹³ molecules/cm², or at least 10¹⁴ molecules/cm². In some embodiments, the density of nonlinear-active labeled protein on the surface may be at most 10¹⁴ molecules/cm², at most 10¹³ molecules/cm², at most 10¹² molecules/cm², at most 10¹¹ molecules/cm², at most 10¹⁰ molecules/cm², at most 10⁹ molecules/cm², at most 10⁸ molecules/cm², at most 10⁷ molecules/cm², at most 10⁶ molecules/cm², at most 10⁵ molecules/cm², at most 10⁴ molecules/cm², at most 10³ molecules/cm², or at most 10² molecules/cm². Those of skill in the art will recognize that the density of nonlinear-active labeled protein on the surface may have any value within this range, for example, about 4.0×10¹² molecules/cm².

High throughput methods, devices, and systems: Also disclosed herein are methods, devices, and systems for implementing high throughput analysis and comparison of structure, conformation, or conformational signatures in biological molecules, e.g., proteins or other biological entities, based on the use of second harmonic generation or related nonlinear optical detection techniques, or on the use of two-photon fluorescence, or on any combination thereof. In some embodiments, the disclosed methods, devices, and systems for high throughput analysis may be used, for example, as screening tools for comparison of candidate biological drugs and reference drugs. As used herein, “high throughput” is a relative term used in comparison to structural measurements performed using traditional techniques such as NMR or X-ray crystallography. As will be described in more detail below, the SHG-based and/or TPF-based methods and systems disclosed herein are capable of performing structural determinations at a rate that is at least an order-of-magnitude faster than that for these conventional techniques.

In some embodiments, determination of biomolecular structure, conformation, or conformational change in a high-throughput format is enabled through the use of novel device designs and mechanisms for rapid, precise, and interchangeable positioning of substrates (comprising the tethered or immobilized biological targets to be analyzed) with respect to the optical system used to deliver excitation light, and which at the same time ensure that efficient optical coupling between the excitation light and the substrate surface is maintained. One preferred format for high-throughput optical interrogation of biological samples is the glass-bottomed microwell plate. The systems and methods disclosed herein provide mechanisms for coupling the high intensity excitation light required for SHG and/or TPF to a substrate, e.g., the glass substrate in a glass-bottomed microwell plate, by means of total internal reflection (TIR) in a manner that is compatible with the requirements for a high-throughput analysis system.

In one aspect, this disclosure provides a method for high throughput detection of structure, conformation, or conformational change in one or more biological molecules (e.g., proteins or other biological entities), the method comprising (i) labeling one or more target biological molecules, e.g. protein molecules, biological drug candidates, or reference drug molecules, with a nonlinear-active label or tag using identical labeling reactions or techniques, (ii) tethering or immobilizing the one or more labeled target biological molecules at one or more discrete regions of a planar substrate surface, wherein the substrate surface further comprises an optical interface, (iii) sequentially exposing each discrete region to excitation light of a fundamental frequency by changing the position of the substrate relative to an external light source, (iv) collecting a nonlinear optical signal (e.g., SHG, SFG, DFG, TPF, or any combination thereof) emitted from each discrete region as it is exposed to excitation light, and (v) processing said nonlinear optical signal to determine an orientation, structure, conformation, or conformational change of each of the one or more target biological molecules. In another aspect, the method further comprises (vi) contacting each of the one or more labeled biological molecules with one or more test entities (e.g., test compounds, candidate binding partners, reference drugs, known ligands, or other controls) following the first exposure to excitation light, (vii) subsequently re-exposing each discrete region to excitation light one or more times, (viii) collecting a nonlinear optical signal from each discrete region as it is exposed to excitation light, and (ix) processing said nonlinear optical signals to determine whether or not a change in orientation or conformation has occurred in the one or more biological entities as a result of contacting with said one or more test entities. In some embodiments, the nonlinear-active label may be attached to the target protein or other biological entity that is tethered or immobilized on a discrete region of the planar substrate. In some embodiments, the nonlinear-active label may be attached to a test entity that is used to contact the tethered or immobilized target protein or other biological entity. In some embodiments, both the tethered or immobilized target protein or other biological entity and the test entity may be labeled with a nonlinear-active tag (i.e., with the same nonlinear-active tag or with a different nonlinear-active tag). In a preferred embodiment the labels are attached to a plurality of unique and different sites on the surface of the target biomolecule (e.g., protein) and the structure, conformation, or conformational change is determined accordingly, i.e. carrying out steps i) through ix) above or some subset of them multiple times with a different site of the protein or target biomolecule labeled in each case and each of these mutants is measured in multiple experiments, each of which has a different experimental condition. For example, if a protein is labeled at 10 different sites with a nonlinear-active label, it may be studied under 50 different experimental conditions in which the underlying orientational distribution in each experiment is different. As a result, 10×50=500 different projections of the transition dipole moment on the normal axis could be determined and this would enable more accurate and more complete determination of the protein's structure and conformational landscape. Moreover, by testing multiple ligands, whether known or not known to bind to the protein, one could generate additional angular information as a function of each ligand binding event—i.e., angular change data due to ligand binding and conformational change, and this information could also be used to provide a more complete determination of the molecule's structure and conformational landscape. In one aspect of the method, nonlinear optical signals are detected only once following contact of the one or more biological entities (target proteins, biological drug candidates, reference drug molecules, etc.) with one or more test entities (i.e. in endpoint assay mode), and then used to determine whether or not conformational change has occurred. In another aspect, nonlinear optical signals are collected repeatedly and at random or defined time intervals following contact of the one of more biological entities with one or more test entities (i.e. in kinetics mode), and then used to determine the kinetics of conformational change in the one or more biological entities.

In a preferred aspect of the method, each discrete region of the substrate comprises a supported lipid bilayer structure, and target proteins or other biological entities are immobilized in each discrete region by means of tethering to or embedding in the lipid bilayer. In another preferred aspect of the method, the excitation light is delivered to the substrate surface, i.e. the optical interface, by means of total internal reflection, and the nonlinear optical signals emitted from the discrete regions of the substrate surface are collected along the same optical axis as the reflected excitation light.

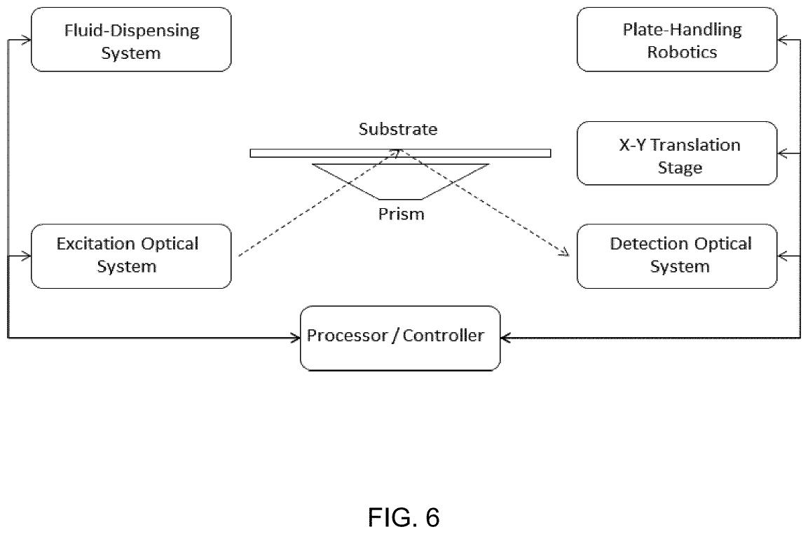

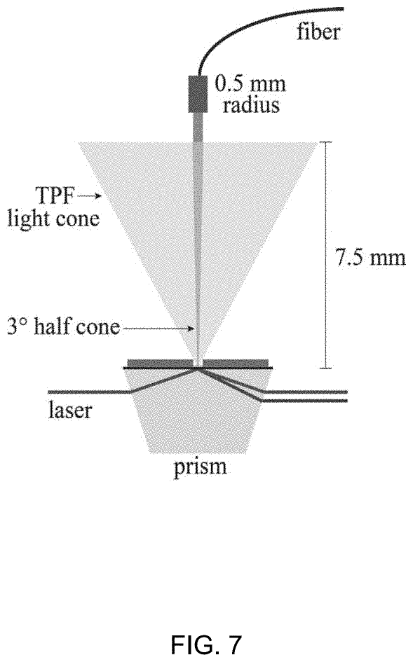

In order to implement high throughput analysis of protein structure, conformation, or conformational change using nonlinear optical detection, the systems described herein require several components (illustrated schematically in FIG. 6), including (i) at least one suitable excitation light source and optics for delivering the at least one excitation light beam to an optical interface, (ii) an interchangeable substrate comprising the optical interface, to which one or more biological entities have been tethered or immobilized in discrete regions of the substrate, (iii) a high-precision translation stage for positioning the substrate relative to the at least one excitation light source, and (iv) optics for collecting nonlinear optical signals generated as a result of illuminating each of the discrete regions of the substrate with excitation light and delivering said nonlinear signals to a detector, and (v) a processor for analyzing the nonlinear optical signal data received from the detector and determining structure, conformation, or conformational change for the one or more biological entities immobilized on the substrate. In some aspects, the systems and methods disclosed herein further comprise the use of (vi) a programmable fluid-dispensing system for delivering test entities to each of the discrete regions of the substrate, and (vii) the use of plate-handling robotics for automated positioning and replacement of substrates at the interface with the optical system. In a preferred embodiment, a relatively low-NA detection scheme is used for the detection of two-photon fluorescence, wherein the optical detector is positioned directly above or below the sample to be measured (e.g., the laser focal spot) along the axis normal to the surface to which the sample is attached. The optical detector should be a distance from the surface and subtend a relatively small acceptance angle such that the NA be as low as possible while still achieving sufficient signal for measurements. In one embodiment, shown in FIG. 7, the detector is a multimode plastic optical fiber with a fiber radius of 0.5 mm whose distance from the slide surface is 7.5 mm. If the TPF signal originates from a discrete region that contains an aqueous environment extending 2 mm from the optical interface before transitioning to air, the resulting detection NA will be 0.054.

The methods, devices, and systems disclosed herein may be configured for analysis of a single biological entity (e.g., a protein, a biological drug candidate, reference drug, or drug target) optionally contacted with a plurality of drug candidates or other test entities (e.g., reference drugs, known ligands, controls, etc.), or for analysis of a plurality of biological entities contacted with a single test entity, or any combination thereof. When contacting one or more biological entities with a plurality of test entities, the contacting step may be performed sequentially, i.e. by exposing the immobilized biological entity to a single test entity for a specified period of time, followed by an optional rinse step to remove the test entity solution and regenerate the immobilized biological entity prior to introducing to the next test entity, or the contacting step may be performed in parallel, i.e. by having a plurality of discrete regions comprising the same immobilized biological entity, and exposing the biological entity in each of the plurality of discrete regions to a different test entity. The methods, devices, and systems disclosed herein may be configured to perform analysis of structure, conformation, or conformational change in at least one biological entity, at least two biological entities, at least four biological entities, at least six biological entities, at least eight biological entities, at least ten biological entities, at least fifteen biological entities, or at least twenty biological entities. In some aspects, methods, devices, and systems disclosed herein may be configured to perform analysis of structure, conformation, or conformational change in at most twenty biological entities, at most fifteen biological entities, at most ten biological entities, at most eight biological entities, at most six biological entities, at most four biological entities, at most two biological entities, or at most one biological entity. Similarly, the methods, devices, and systems disclosed herein may be configured to perform analysis of structure, conformation, or conformational change upon exposure of the one or more biological entities to at least 1 test entity, at least 5 test entities, at least 10 test entities, at least 50 test entities, at least 100 test entities, at least 500 test entities, at least 1,000 test entities, at least 5,000 test entities, at least 10,000 test entities, or at least 100,000 test entities. In some aspects, the methods, devices, and systems disclosed herein may be configured to perform analysis of structure, conformation, or conformational change upon exposure of the one or more biological test entities to at most 100,000 test entities, at most 10,000 test entities, at most 5,000 test entities, at most 1,000 test entities, at most 500 test entities, at most 100 test entities, at most 50 test entities, at most 10 test entities, at most 5 test entities, or at most 1 test entity.

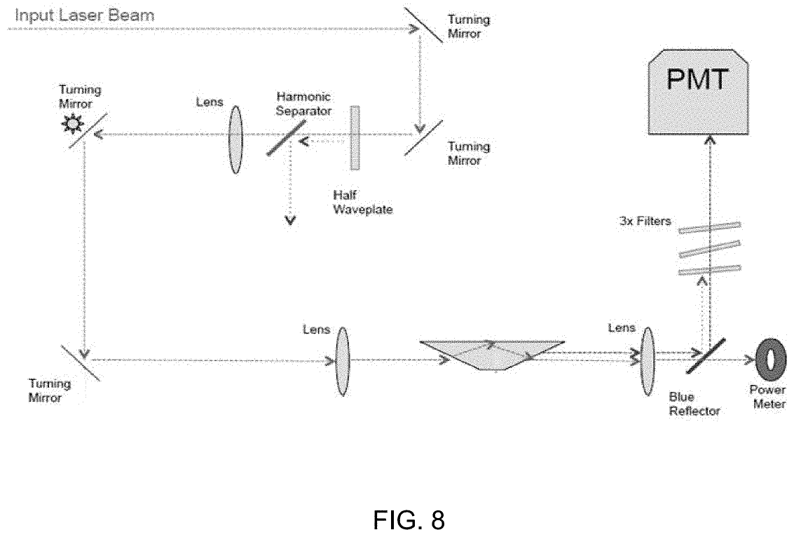

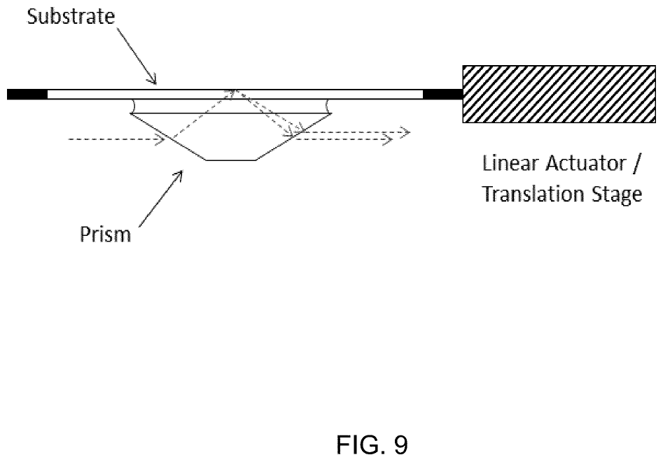

Laser light sources and excitation optical system: FIG. 8 illustrates one aspect of the methods and systems disclosed herein wherein second harmonic light (and/or two-photon fluorescence in some embodiments; FIG. 8 illustrates an optical system for detection of SHG) is generated by reflecting incident fundamental excitation light from the surface of a substrate comprising the sample interface (or optical interface). In some embodiments, the substrate is optically-coupled to a prism used to deliver laser light at the appropriate angle to induce total internal reflection at the substrate surface (FIG. 9). In some embodiments, the optical coupling is provided by use of a thin film of an index-matching fluid. A laser provides the fundamental light necessary to generate second harmonic and fluorescence light at the sample interface. Typically this will be a picosecond or femtosecond laser, either wavelength tunable or not tunable, and commercially available (e.g. a Ti: Sapphire femtosecond laser or fiber laser system). Light at the fundamental frequency (w) exits the laser and its polarization is selected using, for example a half-wave plate appropriate to the frequency and intensity of the light (e.g., available from Melles Griot, Oriel, or Newport Corp.). The beam then passes through a harmonic separator designed to pass the fundamental light but block nonlinear light (e.g. second harmonic light). This filter is used to prevent back-reflection of the second harmonic beam into the laser cavity which can cause disturbances in the lasing properties. A combination of mirrors and lenses are then used to steer and shape the beam prior to reflection from a final mirror that directs the beam via a prism to impinge at a specific location and with a specific angle θ on the substrate surface such that it undergoes total internal reflection at the substrate surface. One of the mirrors in the optical path can be scanned if required using a galvanometer-controlled mirror scanner, a rotating polygonal mirror scanner, a Bragg diffractor, acousto-optic deflector, or other means known in the art to allow control of a mirror's position. The substrate comprising the optical interface and nonlinear-active sample surface can be mounted on an x-y translation stage (computer controlled) to select a specific location on the substrate surface for generation of the second harmonic beam and/or two-photon fluorescence. In some aspects of the methods and systems presently described, it is desirable to scan or rotate one mirror in order to slightly vary the angle of incidence for total internal reflection, and thereby maximize the nonlinear optical signals emitted from the discrete regions of the substrate surface without substantially changing the position of the illuminating excitation light spot. In some aspects, two (or more) lasers having different fundamental frequencies may be used to generate sum frequency or difference frequency light at the optical interface on which the non-linear active sample is immobilized. In some embodiments, the optical excitation may further comprise an additional light source (e.g., a laser, arc lamp, tungsten halogen lamp, or high intensity LED) that is optionally used to excite the intrinsic fluorescence or two-photon fluorescence of the nonlinear-active label (or of an additional fluorescent label attached to the immobilized protein).

Substrate formats, optical interface, and total internal reflection: As described above, the methods, devices, and systems of the present disclosure utilize a planar substrate for tethering or immobilization of one or more biological entities, e.g., target proteins, on a top surface of the substrate, wherein the top substrate surface further comprises the optical interface (or sample interface) used for exciting nonlinear optical signals. The substrate can be glass, silica, fused-silica, plastic, or any other solid material that is transparent to the fundamental and second harmonic light beams, and that supports total internal reflection at the substrate/sample interface when the excitation light is incident at an appropriate angle. In some aspects of the invention, the discrete regions within which biological entities are contained are configured as one-dimensional or two-dimensional arrays, and are separated from one another by means of a hydrophobic coating or thin metal layer. In other aspects, the discrete regions may comprise indents in the substrate surface. In still other aspects, the discrete regions may be separated from each other by means of a well-forming component such that the substrate forms the bottom of a microwell plate (or microplate), and each individual discrete region forms the bottom of one well in the microwell plate. In one aspect of the present disclosure, the well-forming component separates the top surface of the substrate into 96 separate wells. In another aspect, the well-forming component separates the top surface of the substrate into 384 wells. In yet another aspect, the well-forming component separates the top surface of the substrate into 1,536 wells. In all of these aspects, the substrate, whether configured in a planar array, indented array, or microwell plate format, may comprise a disposable or consumable device or cartridge that interfaces with other optical and mechanical components of the measurement system or high throughput system.

The methods, devices, and systems disclosed herein further comprise specifying the number of discrete regions or wells into which the substrate surface is divided, irrespective of how separation is maintained between discrete regions or wells. Having larger numbers of discrete regions or wells on a substrate may be advantageous in terms of increasing the sample analysis throughput of the method or system. In one aspect of the present disclosure, the number of discrete regions or wells per substrate is between 10 and 1,600. In other aspects, the number of discrete regions or wells is at least 10, at least 20, at least 50, at least 100, at least 200, at least 300, at least 400, at least 500, at least 750, at least 1,000, at least 1,250, at least 1,500, or at least 1,600. In yet other aspects of the disclosed methods and systems, the number of discrete regions or wells is at most 1,600, at most 1,500, at most 1,000, at most 750, at most 500, at most 400, at most 300, at most 200, at most 100, at most 50, at most 20, or at most 10. In a preferred aspect, the number of discrete regions or wells is 96. In another preferred aspect, the number of discrete regions or wells is 384. In yet another preferred aspect, the number of discrete regions or wells is 1,536. Those of skill in the art will appreciate that the number of discrete regions or wells may fall within any range bounded by any of these values (e.g. from about 12 to about 1,400).

The methods, devices, and systems disclosed herein also comprise specifying the surface area of the discrete regions or wells into which the substrate surface is divided, irrespective of how separation is maintained between discrete regions or wells. Having discrete regions or wells of larger area may facilitate ease-of-access and manipulation of the associated biological entities in some cases, whereas having discrete regions or wells of smaller area may be advantageous in terms of reducing assay reagent volume requirements and increasing the sample analysis throughput of the method or system. In one aspect of the present disclosure, the surface area of the discrete regions or wells is between 1 mm² and 100 mm². In other aspects, the area of the discrete regions or wells is at least 1 mm², at least 2.5 mm², at least 5 mm², at least 10 mm², at least 20 mm², at least 30 mm², at least 40 mm², at least 50 mm², at least 75 mm², or at least 100 mm². In yet other aspects of the disclosed methods and systems, the area of the discrete regions or wells is at most 100 mm², at most 75 mm², at most 50 mm², at most 40 mm², at most 30 mm², at most 20 mm², at most 10 mm², at most 5 mm², at most 2.5 mm², or at most 1 mm². In a preferred aspect, the area of discrete regions or wells is about 35 mm². In another preferred aspect, the area of the discrete regions or wells is about 8.6 mm². Those of skill in the art will appreciate that the area of the discrete regions or wells may fall within any range bounded by any of these values (e.g. from about 2 mm² to about 95 mm²).

Discrete regions of the substrate surface are sequentially exposed to (illuminated with) excitation light by re-positioning the substrate relative to the excitation light source. Total internal reflection of the incident excitation light creates an “evanescent wave” at the sample interface, which excites the nonlinear-active label and results in generation of second harmonic and fluorescence light (or in some aspects, sum frequency or difference frequency light). Because the intensity of the evanescent wave, and hence the intensity of the nonlinear optical signals generated, is dependent on the incident angle of the excitation light beam, precise orientation of the substrate plane with respect to the optical axis of the excitation beam and efficient optical coupling of the beam to the substrate is critical for achieving optimal SHG and/or TPF signals across the array of discrete regions. In some aspects of the present disclosure, total internal reflection is achieved by means of a single reflection of the excitation light from the substrate surface. In other aspects, the substrate may be configured as a waveguide such that the excitation light undergoes multiple total internal reflections as it propagates along the waveguide. In yet other aspects, the substrate may be configured as a zero-mode waveguide, wherein an evanescent field is created by means of nanofabricated structures.

Efficient optical coupling between the excitation light beam and the substrate in an optical setup such as the one illustrated in FIG. 8 and FIG. 9 would typically be achieved by use of an index-matching fluid such as mineral oil, mixtures of mineral oil and hydrogenated terphenyls, perfluorocarbon fluids, glycerin, glycerol, or similar fluids having a refractive index near 1.5, wherein the index-matching fluid is wicked between the prism and the lower surface of the substrate. Since a static, bubble-free film of index-matching fluid is likely to be disrupted during fast re-positioning of the substrate, the methods, devices, and systems disclosed herein include alternative approaches for creating efficient optical coupling of the excitation beam to the substrate in high throughput systems.

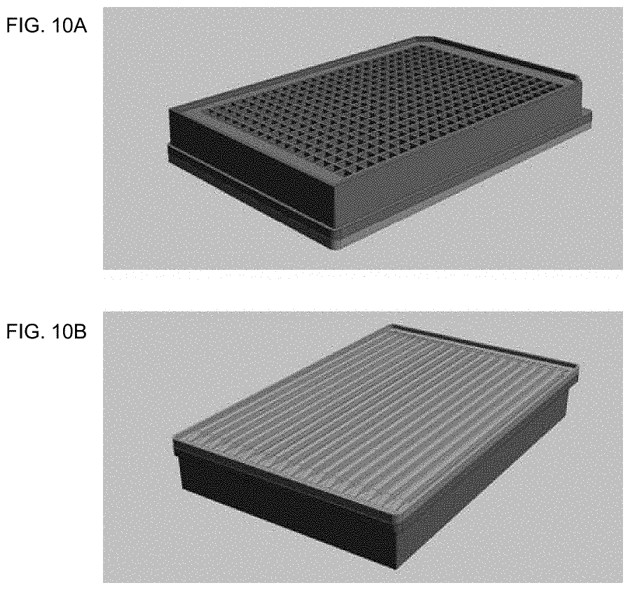

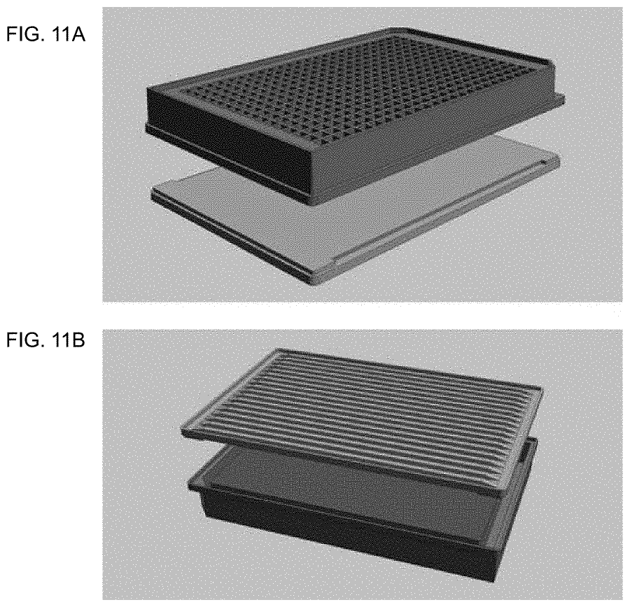

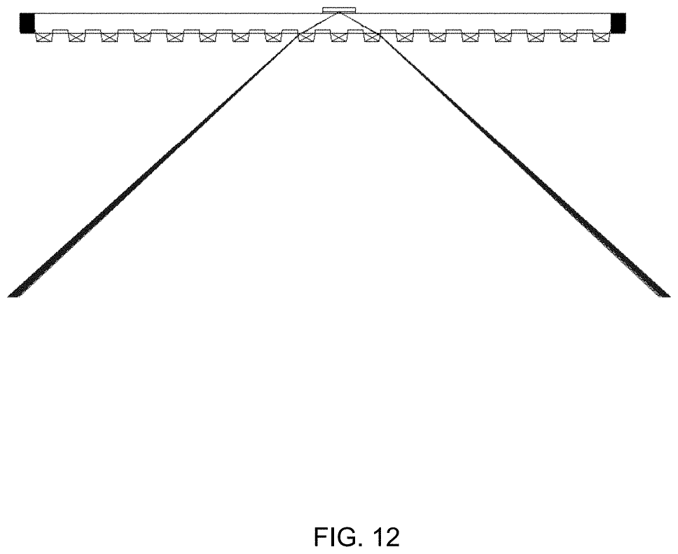

FIGS. 10A-B and FIGS. 11A-B illustrate a preferred aspect of a high throughput system of the present disclosure, in which an array of prisms or gratings is integrated with the lower surface of the substrate (packaged in a microwell plate format) and used to replace the fixed prism, thereby eliminating the need for index-matching fluids or elastomeric layers entirely. The array of prisms (or gratings) is aligned with the array of discrete regions or wells on the upper surface of the substrate in such a way that incident excitation light is directed by an “entrance prism” (“entrance grating”) to a discrete region or well that is adjacent to but not directly above the entrance prism (entrance grating), at an angle of incidence that enables total internal reflection of the excitation light beam from the sample interface (see FIG. 12), and such that the reflected excitation beam, and nonlinear-optical signals generated at the illuminated discrete region, are collected by an “exit prism” (“exit grating”) that is again offset from (adjacent to but not directly underneath) the discrete region under interrogation, and wherein the entrance prism and exit prism (entrance grating and exit grating) for each discrete region are different, non-unique elements of the array.

In general, for an array of discrete regions comprising M rows×N columns of individual features, the corresponding prism or grating array will have M+2 rows×N columns or N+2 columns×M rows of individual prisms or gratings. In some embodiments, for an array of discrete regions comprising M rows×N columns of individual features, the corresponding prism or grating array will have M+4 rows×N columns or N+4 columns×M rows of individual prisms or gratings. In general, M≠N. In some embodiments, M may have a value of at least 2, at least 4, at least 6, at least 8, at least 12, at least 14, at least 16, at least 18, at least 20, at least 25, at least 30, at least 35, at least 40, at least 45, or at least 50 rows. In some embodiments, M may have a value of at most 50, at most 45, at most 40, at most 35, at most 30, at most 25, at most 20, at most 18, at most 16, at most 14, at most 12, at most 10, at most 8, at most 6, at most 4, or at most 2 rows. Similarly, in some embodiments, N may have a value of at least 2, at least 4, at least 6, at least 8, at least 12, at least 14, at least 16, at least 18, at least 20, at least 25, at least 30, at least 35, at least 40, at least 45, or at least 50 columns. In some embodiments, N may have a value of at most 50, at most 45, at most 40, at most 35, at most 30, at most 25, at most 20, at most 18, at most 16, at most 14, at most 12, at most 10, at most 8, at most 6, at most 4, or at most 2 columns. As will be apparent to those of skill in the art, M and N may have the same value or different values, and may have any value within the range specified above, for example, M=15 and N=45.

The geometry and dimensions of the individual prisms or gratings, including the thickness of the prism or grating array layer, are optimized to ensure that incident light undergoes total internal reflection at the selected discrete region of the substrate, and nonlinear optical signals generated at the selected discrete region are collected, with high optical coupling efficiency, independently of the position of substrate (microwell plate) relative to the excitation light beam. The prism or grating arrays may be fabricated by a variety of techniques known to those of skill in the art, for example, in a preferred aspect, they may be injection molded from smooth flowing, low birefringence materials such as cyclic olefin copolymer (COC) or cyclic olefin polymer (COP), acrylic, polyester, or similar polymers. In some aspects, the prism or grating array may be fabricated as a separate component, and subsequently integrated with the lower surface of the substrate. In other aspects, the prism or grating array may be fabricated as an integral feature of substrate itself.

Collection optics and detector: FIG. 8 further illustrates the collection optics and detector used to detect SHG and related nonlinear optical signals generated upon sequential illumination of the discrete regions of the substrate. Because surface-selective nonlinear optical techniques are coherent techniques, meaning that the fundamental and nonlinear optical light beams have wave fronts that propagate through space with well-defined spatial and phase relationships, minimal collection optics are required. Emitted nonlinear optical signals are collected by means of a prism (or the integrated prism or grating array of the microplate device described above) and directed via a dichroic reflector and mirror to the detector. Additional optical components, e.g. lenses, optical bandpass filters, mirrors, etc. are optionally used to further shape, steer, and/or filter the beam prior to reaching the detector. A variety of different photodetectors may be used, including but not limited to photodiodes, avalanche photodiodes, photomultipliers, CMOS sensors, or CCD devices. In some embodiment, the collection optical pathway may further comprise an addition photodetector that is optionally used to detect the intrinsic fluorescence or two-photon fluorescence of the protein or nonlinear-active label (or of an additional fluorescent label attached to the immobilized protein).

Likewise, the collection of two-photon fluorescence signals requires a minimal number of optics in the low-NA limit. A small diameter optical fiber, for instance, when positioned a suitable distance away from the sample can act as a pinhole, collecting a small fraction of light emitted from probe molecules and increasing the sensitivity of the technique. After passing through the fiber, an optical filter can be used to select the appropriate bandwidth corresponding to fluorescence while rejecting background light. As with SHG, a variety of different photodetectors may be used to detect the TPF signal, including but not limited to photodiodes, avalanche photodiodes, photomultipliers, CMOS sensors, or CCD devices.

X-Y translation stage: As illustrated in FIG. 6, implementation of the high throughput systems disclosed herein ideally utilizes a high precision X-Y (or in some cases, an X-Y-Z) translation stage for re-positioning the substrate (in any of the formats described above) in relation to the excitation light beam. Suitable translation stages are commercially available from a number of vendors, for example, Parker Hannifin. Precision translation stage systems typically comprise a combination of several components including, but not limited to, linear actuators, optical encoders, servo and/or stepper motors, and motor controllers or drive units. High precision and repeatability of stage movement is required for the systems and methods disclosed herein in order to ensure accurate measurements of nonlinear optical signals when interspersing repeated steps of optical detection and/or liquid-dispensing. Also, as the size of the focal spot for the excitation light [20-200 microns in diameter or on a side] is substantially smaller than the size of the discrete regions on the substrate, in some aspects of the present disclosure, it may also be desirable to return to a slightly different position within a given discrete region when making replicate measurements, or to slowly scan the excitation beam across a portion of the discrete region over the course of a single measurement, thereby eliminating potential concerns regarding the photo-bleaching effects of long exposures or prior exposures.

Consequently, the methods and systems disclosed herein further comprise specifying the precision with which the translation stage is capable of positioning a substrate in relation to the excitation light beam. In one aspect of the present disclosure, the precision of the translation stage is between about 1 um and about 10 um. In other aspects, the precision of the translation stage is about 10 um or less, about 9 um or less, about 8 um or less, about 7 um or less, about 6 um or less, about 5 um or less, about 4 um or less, about 3 um or less, about 2 um or less, or about 1 um or less. Those of skill in the art will appreciate that the precision of the translation stage may fall within any range bounded by any of these values (e.g. from about 1.5 um to about 7.5 um).

Fluid dispensing system: As illustrated in FIG. 6, some embodiments of the high throughput systems disclosed herein further comprise an automated, programmable fluid-dispensing (or liquid-dispensing) system for use in contacting the biological or target entities immobilized on the substrate surface with test entities (or test compounds), the latter typically being dispensed in solutions comprising aqueous buffers with or without the addition of a small organic solvent component, e.g. dimethylsulfoxide (DMSO). Suitable automated, programmable fluid-dispensing systems are commercially available from a number of vendors, e.g. Beckman Coulter, Perkin Elmer, Tecan, Velocity 11, and many others. In a preferred aspect of the systems and methods disclosed herein, the fluid-dispensing system further comprises a multichannel dispense head, e.g. a 4 channel, 8 channel, 16 channel, 96 channel, or 384 channel dispense head, for simultaneous delivery of programmable volumes of liquid (e.g. ranging from about 1 microliter to several milliliters) to multiple wells or locations on the substrate.

Plate-handling robotics: In other aspects of the high throughput systems disclosed herein, the system further comprises a microplate-handling (or plate-handling) robotic system (FIG. 6) for automated replacement and positioning of substrates (in any of the formats described above) in relation to the optical excitation and detection optics, or for optionally moving substrates between the optical instrument and the fluid-dispensing system. Suitable automated, programmable microplate-handling robotic systems are commercially available from a number of vendors, including Beckman Coulter, Perkin Elemer, Tecan, Velocity 11, and many others. In a preferred aspect of the systems and methods disclosed herein, the automated microplate-handling robotic system is configured to move collections of microwell plates comprising immobilized biological entities and/or aliquots of test compounds to and from refrigerated storage units.

Processor/controller and constraint-based scheduling algorithm: In another aspect of the present disclosure, the high throughput systems disclosed further comprise a processor (or “controller” or “computer”) (FIG. 6) configured to run system software which may optionally be stored on a memory unit and which controls the various subsystems described (excitation and detection optical systems, X-Y (or X-Y-Z) translation stage, fluid-dispensing system, and plate-handling robotics) and synchronizes the different operational steps involved in performing high throughput SHG and/or SHG-to-TRPF signal ratio measurements and analysis. In addition to handling the data acquisition process, i.e. collection of output electronic signals from the detector that correspond to the nonlinear optical signals associated with conformational change, the processor or controller is also typically configured to store the data, perform data processing and display functions (including determination of whether or not changes in baseline signals, orientation, or conformation have occurred for the biological entities, or combinations of biological and test entities, that have been tested), and operate a graphical user interface for interactive control by an operator. The processor or controller may also be networked with other processors, or connected to the internet for communication with other instruments and computers at remote locations.

Typical input parameters for the processor/controller may include set-up parameters such as the total number of microwell plates to be analyzed; the number of wells per plate; the number of times excitation and detection steps are to be performed for each discrete region of the substrate or well of the microplate (e.g. to specify endpoint assay or kinetic assay modes); the total time course over which kinetic data should be collected for each discrete region or well; the order, timing, and volume of test compound solutions to be delivered to each discrete region or well; the dwell time for collection and integration of nonlinear optical signals; the name(s) of output data files; and any of a number of system set-up and control parameters known to those skilled in the art.

In a preferred aspect of the present disclosure, the processor or controller is further configured to perform system throughput optimization by means of executing a constraint-based scheduling algorithm. This algorithm utilizes system set-up parameters as described above to determine an optimal sequence of interspersed excitation/detection and liquid-dispensing steps for discrete regions or wells that may or may not be adjacent to each other, such that the overall throughput of the system, in terms of number of biological entities and/or test entities analyzed per hour, is maximized. Optimization of system operational steps is an important aspect of achieving high throughput analysis. In some aspects of the disclosed methods and systems, the average throughput of the analysis system may range from about 10 test entities tested per hour to about 1,000 test entities tested per hour. In some aspects, the average throughput of the analysis system may be at least 10 test entities tested per hour, at least 25 test entities tested per hour, at least 50 test entities tested per hour, at least 75 test entities tested per hour, at least 100 test entities tested per hour, at least 200 test entities tested per hour, at least 400 test entities tested per hour, at least 600 test entities tested per hour, at least 800 test entities tested per hour, or at least 1,000 test entities tested per hour. In other aspects, the average throughput of the analysis system may be at most 1,000 test entities tested per hour, at most 800 test entities tested per hour, at most 600 test entities tested per hour, at most 400 test entities tested per hour, at most 200 test entities tested per hour, at most 100 test entities tested per hour, at most 75 test entities tested per hour, at most 50 test entities tested per hour, at most 25 test entities tested per hour, or at most 10 test entities tested per hour.

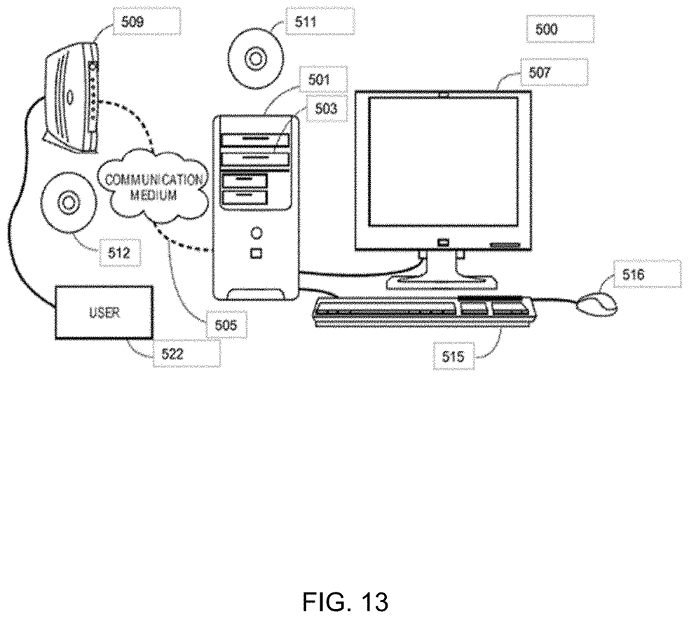

Computer systems and networks: In various embodiments, the methods and systems of the invention may further comprise software programs installed on computer systems and use thereof. Accordingly, as noted above, computerized control of the various subsystems and synchronization of the different operational steps involved in performing high throughput conformational analysis, including data analysis and display, are within the bounds of the invention. The computer system 500 illustrated in FIG. 13 may be understood as a logical apparatus that can read instructions from media 511 and/or a network port 505, which can optionally be connected to server 509 having fixed media 512. The system, such as shown in FIG. 13 can include a CPU 501, disk drives 503, optional input devices such as keyboard 515 and/or mouse 516 and optional monitor 507. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 522 as illustrated in FIG. 13.

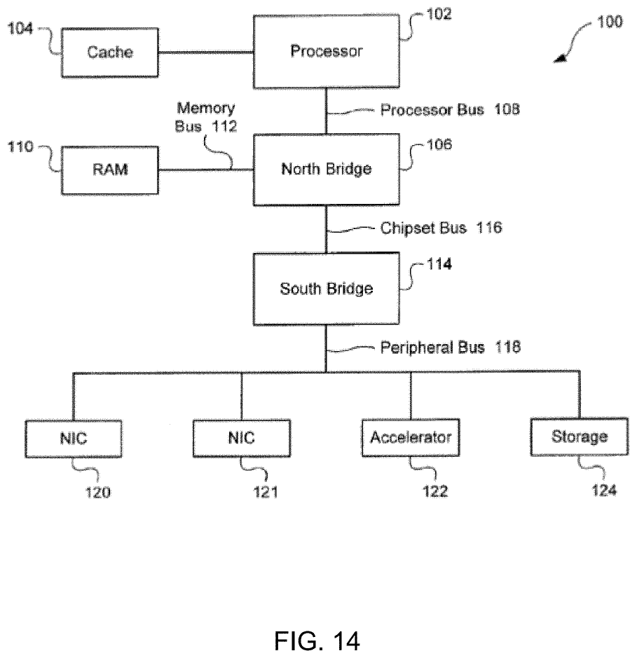

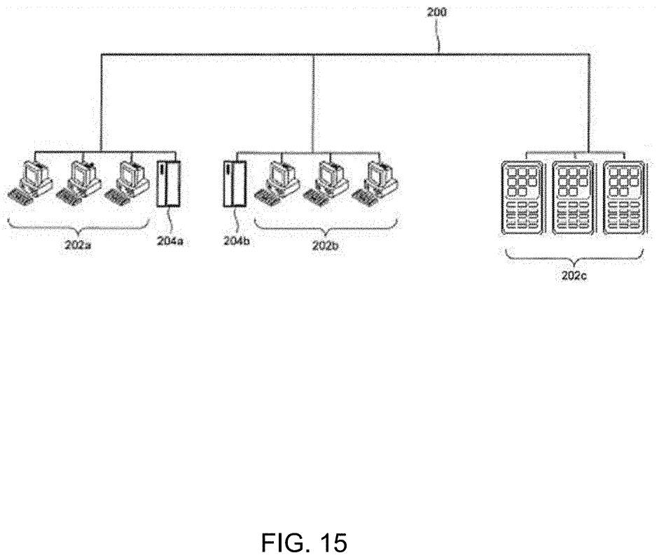

FIG. 14 is a block diagram illustrating a first example architecture of a computer system 100 that can be used in connection with example embodiments of the present invention. As depicted in FIG. 14, the example computer system can include a processor 102 for processing instructions. Non-limiting examples of processors include: the Intel Xeon™ processor, the AMD Opteron™ processor, the Samsung 32-bit RISC ARM 1176JZ(F)-S v1.0™ processor, the ARM Cortex-A8 Samsung S5PC100™ processor, the ARM Cortex-A8 Apple A4™ processor, the Marvell PXA 930™ processor, or a functionally-equivalent processor. Multiple threads of execution can be used for parallel processing. In some embodiments, multiple processors or processors with multiple cores can also be used, whether in a single computer system, in a cluster, or distributed across systems over a network comprising a plurality of computers, cell phones, and/or personal data assistant devices.

As illustrated in FIG. 14, a high speed cache 104 can be connected to, or incorporated in, the processor 102 to provide a high speed memory for instructions or data that have been recently, or are frequently, used by processor 102. The processor 102is connected to a north bridge 106 by a processor bus 108. The north bridge 106 is connected to random access memory (RAM) 110 by a memory bus 112 and manages access to the RAM 110 by the processor 102. The north bridge 106 is also connected to a south bridge 114 by a chipset bus 116. The south bridge 114 is, in turn, connected to a peripheral bus 118. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 118. In some alternative architectures, the functionality of the north bridge can be incorporated into the processor instead of using a separate north bridge chip.

In some embodiments, system 100 can include an accelerator card 122 attached to the peripheral bus 118. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 124 and can be loaded into RAM 110 and/or cache 104 for use by the processor. The system 100 includes an operating system for managing system resources; non-limiting examples of operating systems include: Linux, Windows™, MacOS™, BlackBerry OS.™, iOS™, and other functionally-equivalent operating systems, as well as application software running on top of the operating system for managing data storage and optimization in accordance with example embodiments of the present invention.

In this example, system 100 also includes network interface cards (NICs) 120 and 121 connected to the peripheral bus for providing network interfaces to external storage, such as Network Attached Storage (NAS) and other computer systems that can be used for distributed parallel processing.

FIG. 15 is a diagram showing a network 200 with a plurality of computer systems 202a, and 202b, a plurality of cell phones and personal data assistants 202c, and Network Attached Storage (NAS) 204a, and 204b. In example embodiments, systems 202a, 202b, and 202c can manage data storage and optimize data access for data stored in Network Attached Storage (NAS) 204a and 204b. A mathematical model can be used for the data and be evaluated using distributed parallel processing across computer systems 202a, and 202b, and cell phone and personal data assistant systems 202c. Computer systems 202a, and 202b, and cell phone and personal data assistant systems 202c can also provide parallel processing for adaptive data restructuring of the data stored in Network Attached Storage (NAS) 204a and 204b. FIG. 15 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

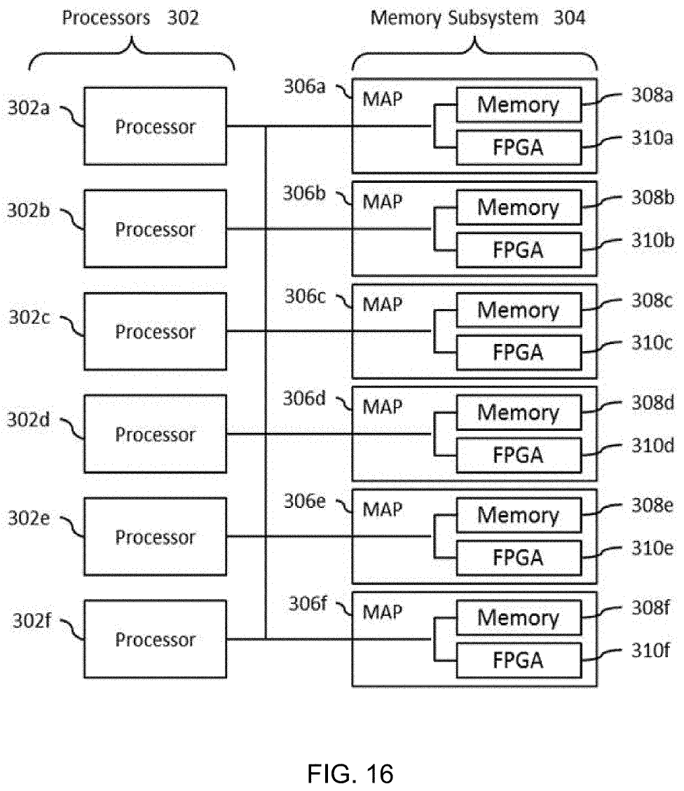

FIG. 16 is a block diagram of a multiprocessor computer system using a shared virtual address memory space in accordance with an example embodiment. The system includes a plurality of processors 302a-f that can access a shared memory subsystem 304. The system incorporates a plurality of programmable hardware memory algorithm processors (MAPs) 306a-f in the memory subsystem 304. Each MAP 306a-f can comprise a memory 308a-f and one or more field programmable gate arrays (FPGAs) 310a-f. The MAP provides a configurable functional unit and particular algorithms or portions of algorithms can be provided to the FPGAs 310a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 308a-f, allowing it to execute tasks independently of, and asynchronously from, the respective microprocessor 302a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs) as referenced in FIG. 16, system on chips (SOCs), application specific integrated circuits (ASICs), or other processing and logic elements. For example, the Set Processor and Optimizer can be implemented with hardware acceleration through the use of a hardware accelerator card, such as accelerator card 122 illustrated in FIG. 14.

### EXAMPLES

These examples are provided for illustrative purposes only and not to limit the scope of the claims provided herein.

### Example 1

### Determination of Structural Parameters in Dihydrofolate Reductase (DHFR) Mutants

Glassware cleaning: Clean all glassware with Piranha wash (20 minutes) prior to starting. Use caution—Piranha wash is highly exothermic and prone to explosion, especially when in contact with organics. Prepare a solution in heat-safe glassware such as Pyrex in a fume hood by measuring out H₂O₂ first, then adding acetic acid.

Sonicated lipid preparation: Rinse vacuum bottles with Chloroform (CHCl3). Determine desired molar ratio of dioleoylphosphatidylcholine (DOPC) lipid to 1,2-dioleoyl-sn-glycero-3-[(N-(5-amino-1-carboxypentyl)iminodiacetic acid)succinyl] (nickel salt) (DGS NTA-Ni) while taking care to avoid exposure to air as much as possible. Place vacuum bottle with lipid mix onto a Rotovap evaporator. Evaporate until dry (about 30 seconds) and then blow N₂ gas over the evaporated preparation for 10 min to remove residual CHCl₃. Resuspend the lipid mixture in 2 mL of diH₂O. Vortex vigorously until a cloudy suspension forms (about 5 minutes). Transfer the suspension to a 4 mL polystyrene test tube. Sonicate the lipid mixture on ice until the solution clears. This should require about 60 to 90 seconds with the sonicator set to 25% power.

Transfer the sonicated lipid solution into microcentrifuge tubes and centrifuge at 17,000×G for 30 minutes at 4° C. Transfer the supernatant into clean microcentrifuge tubes and store the finished lipid preps at 4° C. which are stable for about 1 month.

Slide preparation and protein loading: Immediately before applying DOPC/DGS NTA (Ni) lipids, clean microscope slides with Piranha wash for 30 minutes. Rinse 8× with diH₂O in a slide staining vessel. Dry slides with compressed nitrogen. Assemble SHG wells by attaching adhesive gaskets to Piranha-cleaned slides (i.e., 16 wells per slide containing 10-20 μl volumes each). Use an assembly jig to align gaskets, carefully lay slide into jig and press firmly. Dilute DOPC/DGS NTA (Ni) lipid prep 1:1 with PBS or TBS buffers. 100 mM NaCl is required to reduce hydrostatic charge of the glass slide and enable the supported lipid bilayer (SLB) to form. Pipet 10-20 μL of diluted DOPC/DGS NTA (Ni) lipid into the wells of the slide and incubate for 30 minutes at room temperature. Wash the wells by exchanging the buffer (PBS or TBS) with 20 μL fresh buffer 10× taking care not to introduce air into the wells at any time. If necessary, exchange the buffer in the wells to an appropriate protein loading buffer and load the target protein of interest onto the wells. Incubate for 1 to 24 hours at 4 degrees Celsius followed by a thorough rinse of the wells with assay buffer before starting experiments.

Small unilamellar vesicles (SUVs) are prepared by sonication as described above and applied over Piranha-washed Fisher slides to make the SLB surface. NiCl₂ was added for 10 minutes and wells were washed in labeling buffer.

Labeled protein is loaded onto the SLB surface prepared as describe above at 1 μM (micromolar) for 1 to 24 hours, followed by washing. If imidazole or EDTA is added, or the protein is incubated with the SLB surface in the presence of one or both, the SHG signal drops to the baseline level indicating that attachment to the surface occurs specifically via the protein's His-tag.

A mutant of the Escherichia coli protein dihydrofolate reductase (DHFR) with either an N-terminal or C-terminal 8× His tag was created using methods known to those of skill in the art (Rajagopalan, P., et al. (2002), “Interaction of Dihydrofolate Reductase with Methotrexate: Ensemble and Single-Molecule Kinetics”, Proc. Nat. Acad. of Sci. (USA) 99(21):13481-6; Goodey, N. (2008), “Allosteric Regulation and Catalysis Emerge via a Common Route”, Nat Chem. Biol. 4(8):474-82; Antikainen, N. (2005), “Conformation Coupled Enzyme Catalysis: A Single-Molecule and Transient Kinetics Investigation of Dihydrofolate Reductase”, Biochemistry 44(51):16835-43). In the first case, a cysteine-minimized mutant was made in which both native cysteine residues were removed (C85A and C152A). Then a single, different residue (e.g., M16C, N23C, Q65C, K76C, etc.) was mutated to cysteine, i.e., single-site mutant constructs were created in this cysteine-minimized background. To select the site for the mutation, various residues on the surface of the protein were mutated to cysteine and tested for an ability to be labeled by an SHG probe. In the second case, the wild-type protein was labeled and attachment of the probe to C152 was confirmed by mass spectrometry. Wild type or recombinant protein was purified into 25 mM Tris pH 7.2, 150 mM NaCl. The proteins were labeled using PyMPO maleimide following the manufacturer's instructions, e.g., protein was incubated in 25 mM Tris pH 7.2, 150 mM NaCl, 1 mM TCEP and 10% glycerol at approximately 50 uM with a 20:1 dye:protein labeling ratio (final DMSO concentration was 5%). The protein was stirred overnight at 4° C., and then gel-purified into 25 mM Tris-HCl pH 7.2, 150 mM NaCl, 1 mM TCEP. Labeled and purified protein was then tethered to the SLB surface via the His-tag.

TPF and SHG measurements: Polarization-dependent measurements of the SHG signal were used to determine the independent, non-vanishing components of χ⁽²⁾ for the mutants using methods known to those skilled in the art. For example, in the simplest optical geometry, and assuming azimuthal isotropy and a single dominant component of the hyperpolarizability tensor, one determines two independent non-vanishing components of χ⁽²⁾ (χ_{zzz} and χ_{xzx} or χ_{zxx}). These in turn were used to best determine the orientational distributions related to the ϕ's for each of the mutants (i.e., ϕ₁ and ϕ₂).

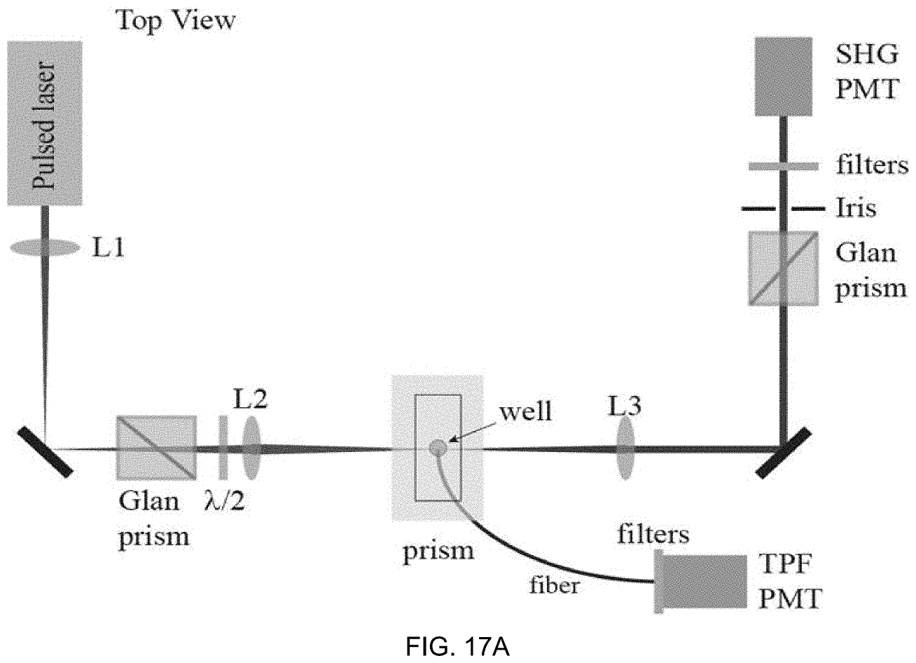

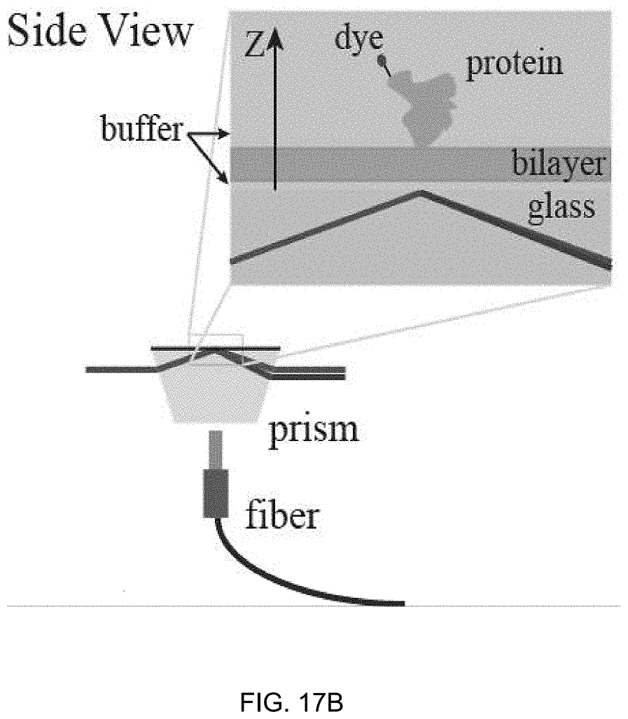

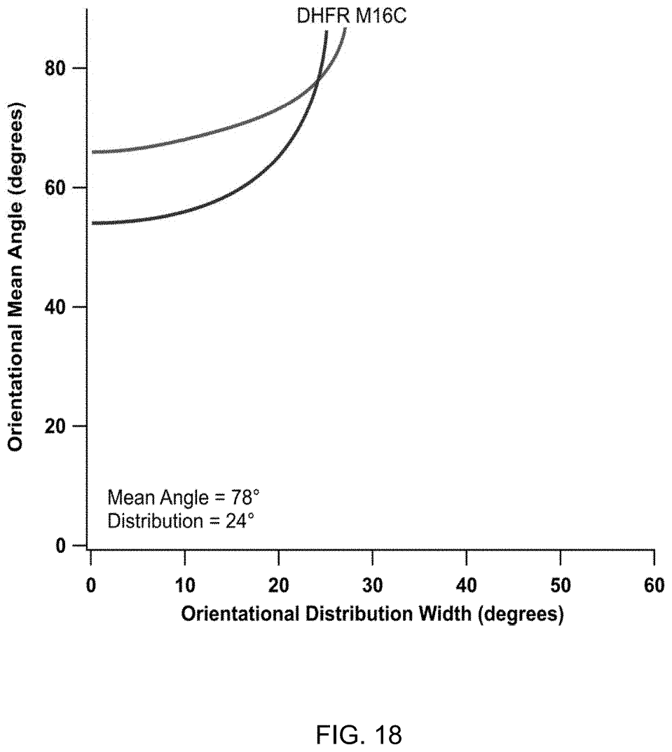

For example, for the M16C mutant, immobilized via a His-tag at the C-terminus, measurements of the TPF and SHG signals were carried out under both p- and s-polarization excitation using the optical setup illustrated in FIGS. 17A-B. The TPF was detected using a pinhole detection apparatus consisting of a fiber optic cable positioned normal to the surface plane and along the axis to the portion of the sample under illumination by the excitation beam (directly above the sample) and a photomultiplier. By combining both the TPF and the SHG measurements and taking ratios of intensities of each under the two different polarizations of excitation, one obtains two independent equations which are only a function of the angular orientational distribution of the probes. Because one has two independent equations using both TPF and SHG measurements, one need not assume a zero-width orientational distribution (i.e., a Delta function distribution), but rather can fit our data to a distribution containing both a mean angle, ϕ, and an orientational width, σ. By assuming a Gaussian orientational distribution for use in fitting the data (i.e., by integrating the left-hand sides of equations (2) and (6) as described above to identify pairs of mean tilt angle and distribution width values that satisfy the equations when the measured values for intensity ratios have been substituted), the unique mean angle and the orientational distribution width for the M16C DHFR mutant was determined from the intersection of the TPF and SHG curve trajectories (TPF=green trace; SHG=blue trace) in the phase space of (ϕ, σ) (FIG. 18), which indicates a pair of ϕ and σ values that are consistent with both the measured TPF and SHG ratios. For the M16C mutant, the mean orientational tilt angle was determined to be 78°, while the width of the orientational distribution was determined to be 24°.

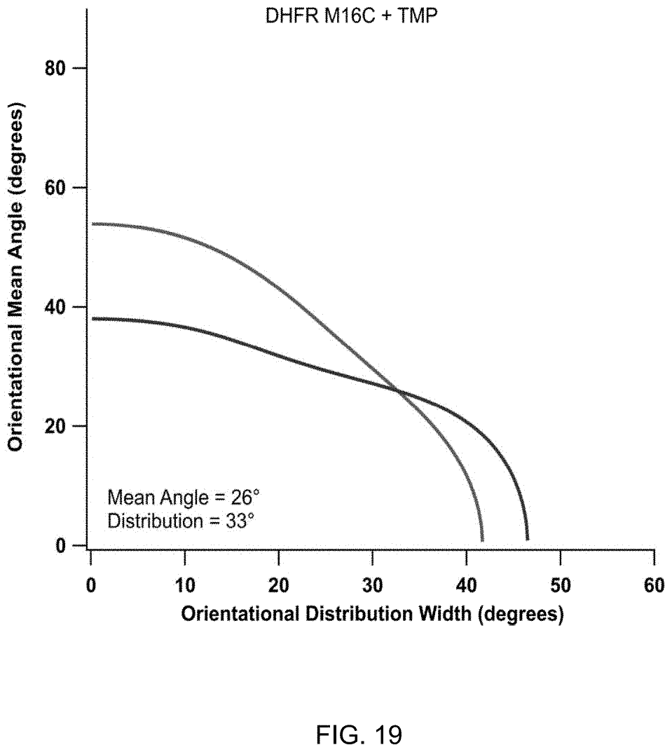

Addition of ligand caused changes in the SHG baseline signal levels (SHG % changes), either positive or negative, which result from reorientation of the labels across the protein ensemble. In other words, the change in SHG baseline signal results from changes in the label orientational distribution as a result of ligand binding. FIG. 19 (TPF curves=green traces; SHG curves=blue traces) shows the results of an experiment in which 1 uM (micromolar) trimethoprim (TMP), a ligand that is known to bind DHFR, produced differential changes in baseline signal for TPF and SHG resulting in a very different mean angle and a broader orientational distribution width for the ligand-bound state. In the presence of 1 μM TMP (FIG. 19), the mean orientational tilt angle was determined to be 26°, while the width of the orientational distribution was determined to be 33°.

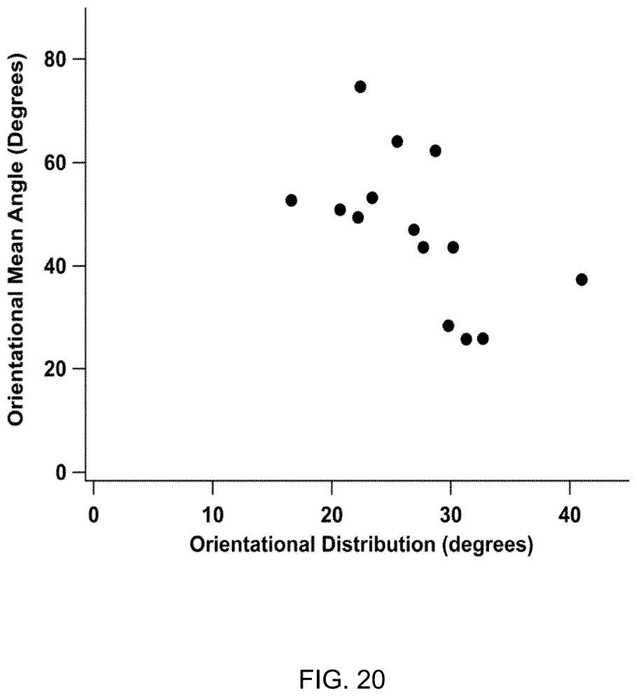

The procedure outlined above for determining the unique orientational mean angle and orientational distribution has been repeated for single-cysteine DHFR mutants labeled at residues M16C, N23C, R52C, Q65C, T73C, K76C, and E118C. Experiments were performed with and without the ligand TMP and the results of those experiments are detailed in Table 4 and FIG. 20. Since the dye is located on a different region of the protein for each single-cysteine DHFR mutant, a different angular response is observed upon binding TMP. In the extreme cases the M16C mutant displays a large conformational rearrangement, whereas in the case of the Q65 mutant the angular changes are more modest. Changes in dye orientation (mean angle) and orientational distribution due to conformational rearrangement of the protein after ligand addition for all single-cysteine mutants are summarized in Table 4 and displayed in FIG. 20.

Orientational mean angle and distribution
measured for each single-cysteine mutant

| | Orientational | Orientational |
|---|---|---|
| DHFR Mutant | Mean Angle | Distribution |
| M16C | 74.70° | 22.40 |
| M16C + TMP | 25.9° | 32.7° |
| N23C | 37.4° | 41.0° |
| N23C + TMP | 47.0° | 26.9° |
| R52C | 52.7° | 16.6° |
| R52C + TMP | 49.4° | 22.2° |
| Q65C | 62.30 | 28.7° |
| Q65C + TMP | 64.1° | 25.50 |
| T73C | 50.9° | 20.7° |
| T73 + TMP | 43.6° | 27.7° |
| K76C | 28.4° | 29.8° |
| K76C + TMP | 25.8° | 31.3° |
| E118C | 43.6° | 30.2° |
| E118C + TMP | 53.2° | 23.4° |

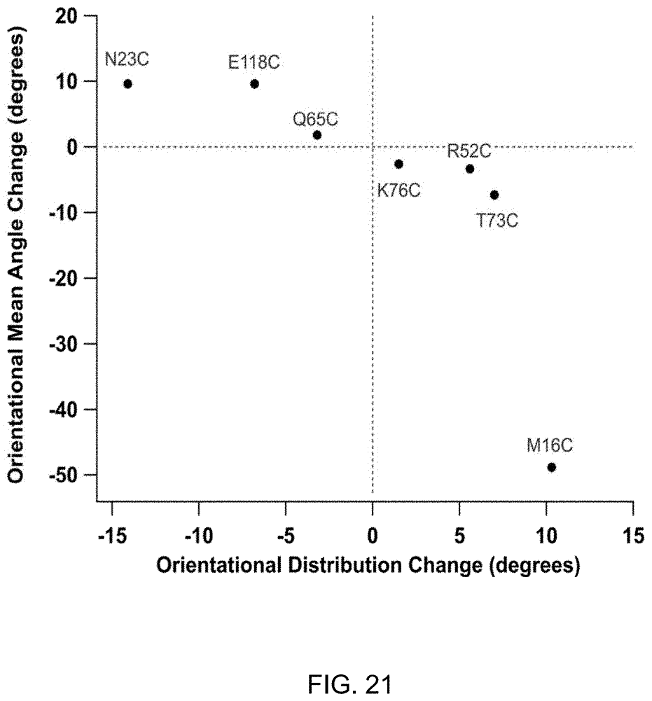

FIG. 21 displays the change in orientational mean angle and change in orientational distribution for each single-cysteine mutant after addition of TMP. The intersection of the dashed lines define an origin of no orientational change. The data indicate that the M16C mutant and the N23C mutant display the largest change in orientational mean angle and orientational distribution upon addition of TMP, respectively.

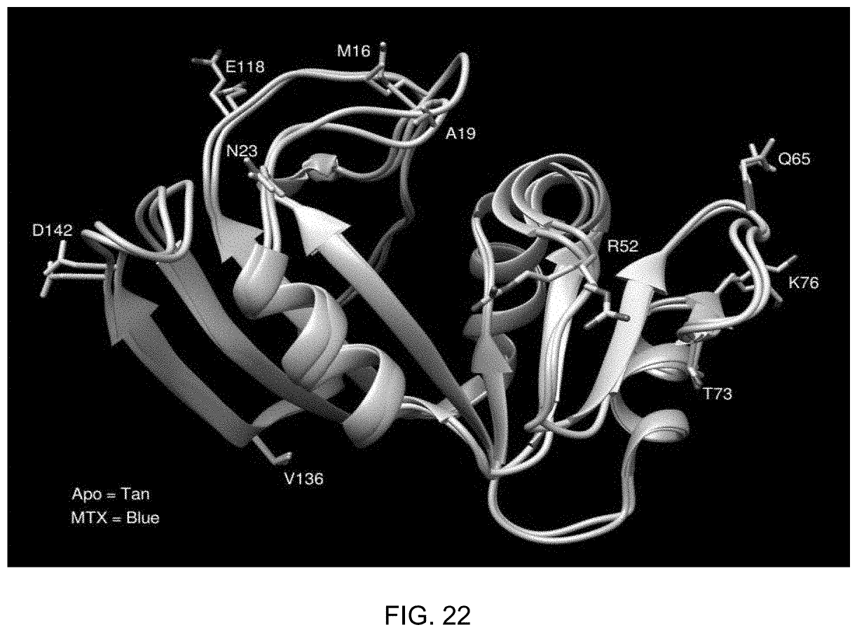

The orientational changes upon ligand addition at the single-cysteine mutated sites measured using the described method can be qualitatively confirmed by comparing published crystal structures in the presence and absence of TMP. FIG. 22 is an overlay of the crystal structure of DHFR in the presence (blue) and absence (tan) of the pharmaceutical inhibitor methotrexate (MTX), which produces conformational changes in DHFR that are analogous to TMP. Locations of the residues that have been mutated into cysteine are labeled in the figure, and the angular orientation of the side chain with respect to the peptide backbone before mutation is shown. Large changes in side chain orientation are observed in FIG. 22 upon addition of MTX to mutant proteins such as the labeled M16C mutant, which is predicted by the described method (see FIG. 21). In contrast, the side chain orientation at some sites, such as Q65C, display little to no angular change in FIG. 22, which is also predicted by the described method (see FIG. 21).

### Example 2

### Determination of Structural Parameters in Protein Labeled with an Unnatural Amino Acid (Prophetic Example)

p17 protein is made in mammalian cells (HEK 293) using L-Anap as a label, an unnatural amino acid that is both SHG- and TPF-active, where the L-Anap is incorporated into the protein using any of a variety of genetic engineering techniques known to those skilled in the art (see, for example, Chatterjee, et al. (2013), A Genetically Encoded Fluorescent Probe in Mammalian Cells, J Am Chem Soc. 135(34):12540-12543; Lee, et al. (2009), The Genetic Incorporation of a Small, Environmentally Sensitive, Fluorescent Probe into Proteins in S. Cerevisiae, J Am Chem Soc. 131(36):12921-12923).

The experimental protocol described in Example 1 is performed using an SHG- and TPF-active unnatural amino acid (e.g., L-Anap or Aladan) as a label instead of an exogenous label. Unnatural amino acid-labeled p17 is then coupled to a phosphatidylserine/DOPC supported lipid bilayer (25%-75% by mole %, respectively) according to procedures known to those skilled in the art (see, for example, Nanda, et al. (2010), Electrostatic Interactions and Binding Orientation of HIV-1 Matrix Studied by Neutron Reflectivity, Biophysical J. 99(8):2516-2524). The mean tilt angle and orientational distribution width of the unnatural amino acid incorporated within p17 (which in turn is attached electrostatically to the supported lipid bilayer) is then measured by determining the intensities of the detected light using a low-NA detection scheme, e.g., without a lens, in both the SHG and TPF channels, under both p- and s-polarized excitation.

### Example 3

### Determination of Structural Parameters Under Different Experimental Conditions (Prophetic Example)

The experiment of Example 1 may be extended to include different concentrations of a molecule added to the buffer (i.e., an additive) (e.g., PEG400 at different concentrations (e.g., 10 uM, 20 uM, 40 uM and 80 uM micromolar PEG400)) which associates with the interfacial region and produces different orientational distributions for the tethered protein. This increases the number of independent molecular orientational distributions and polarization measurements, and thus increases the accuracy of the angular measurements and the protein structural models.

### Example 4

### Determination of Structural Parameters Using Different Mutants of the Target Protein Under Different Experimental Conditions (Prophetic Example)

The experiment of Examples 1 or 2 may be extended to include 10 different mutants of DHFR, each labeled at a different single cysteine site. This increases the number of independent polarization measurements, and thus increases the accuracy of the angular measurements and corresponding protein structural models.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

1. A method for determining angular parameters of a two-photon fluorescent label attached to a tethered biomolecule, the method comprising:
(a) attaching a biomolecule to a planar surface in an oriented manner, wherein the biomolecule is labeled at a known site with a two-photon fluorescent label;
(b) illuminating the attached biomolecule with excitation light of a first fundamental frequency using a first polarization;
(c) detecting a first physical property of light generated by the two-photon fluorescent label as a result of the illumination in step (b);
(d) illuminating the attached biomolecule with excitation light of the first fundamental frequency using a second polarization;
(e) detecting a second physical property of light generated by the two-photon fluorescent label as a result of the illumination in step (d); and
(f) comparing the second physical property of light detected in step (e) to the first physical property of light detected in step (c) to determine angular parameters of the two-photon fluorescent label relative to the planar surface.

2. The method of claim 1, wherein the first physical property is p-polarized light intensity Iₚ and the second physical property is s-polarized intensity Iₛ and the comparison in step (f) comprises solving the following equation to determine angular parameters:
$$\frac{〈 {cos}^{4} {φsin}^{2} φ 〉}{〈 {sin}^{6} φ 〉} = \frac{3}{8} \frac{1}{{f}^{4}} \frac{{I}_{p}}{{I}_{s}} .$$

3. The method of claim 1, further comprising repeating steps (a) through (f) for each of a series of two or more different biomolecule conjugates, wherein each of the biomolecule conjugates in the series comprises the biomolecule labeled at a different site with the same two-photon fluorescent label, and determining a structure of the biomolecule using the angular parameters determined for each of the two or more different biomolecule conjugates.

4. The method of claim 3, wherein the biomolecule is a protein, and wherein the series of two or more different biomolecule conjugates each comprise a single-site cysteine or methionine substitution.

5.-6. (canceled)

7. The method of claim 1, wherein the two-photon fluorescent label is also second harmonic (SH)-active, sum frequency (SF)-active, or difference frequency (DF)-active.

8. The method of claim 7, further comprising simultaneously or subsequently detecting a first physical property of light generated by the second harmonic (SH)-active, sum frequency (SF)-active, or difference frequency (DF)-active label in step (c), and a second physical property of light generated by the second harmonic (SH)-active, sum frequency (SF)-active, or difference frequency (DF)-active label in step (e), upon illumination by excitation light of a second fundamental frequency, where the second fundamental frequency may be the same as or different from the first fundamental frequency.

9. The method of claim 8, further comprising comparing the second physical property of the light detected in step (e) to the first physical property of the light detected in step (c) to determine angular parameters of the second harmonic (SH)-active, sum frequency (SF)-active, or difference frequency (DF)-active label relative to the planar surface.

10. The method of claim 1, further comprising globally fitting data for the angular parameters of one or more two-photon fluorescent labels, second harmonic (SH)-active labels, sum frequency (SF)-active labels, or difference frequency (DF)-active labels, or any combination thereof, to a structural model of the biomolecule, wherein the structural model comprises information about the known sites of the one or more labels within the biomolecule.

11. (canceled)

12. The method of claim 1, wherein the biomolecule is a protein, and wherein the two-photon fluorescent label is a nonlinear-active unnatural amino acid.

13. (canceled)

14. The method of claim 12, wherein the nonlinear-active unnatural amino acid comprises a nonlinear-active moiety attached to an unnatural amino acid that is not appreciably nonlinear-active.

15.-20. (canceled)

21. The method of claim 1, wherein the first and second physical properties of light are an intensity or a polarization.

22. The method of claim 1, wherein the light generated by the two-photon fluorescent label is detected using a low numerical aperture pinhole configuration without the use of a collection lens.

23. (canceled)

24. The method of claim 1, wherein the planar surface comprises a supported lipid bilayer and the biomolecules are attached to or inserted into the supported lipid bilayer.

25. The method of claim 1, wherein the excitation light is directed to the planar surface using total internal reflection.

26. The method of claim 1, wherein the two-photon fluorescent label is also second harmonic (SH)-active, sum frequency (SF)-active, or difference frequency (DF)-active, and further comprising determining angular parameters of the label by:
(g) simultaneously or sequentially detecting an intensity of light generated by the second harmonic (SH)-active, sum frequency (SF)-active, or difference frequency (DF)-active label attached to the attached biomolecule upon illumination with excitation light of a second fundamental frequency which may be the same as or different from the excitation light of the first fundamental frequency, and wherein detection is performed using:
(i) a first polarization state of the excitation light; and
(ii) a second polarization state of the excitation light;
(h) determining angular parameters of a second harmonic (SH)-active, sum frequency (SF)-active, or difference frequency (DF)-active label relative to a normal to the substrate surface by calculating a ratio of the light intensities detected in in step (c)(i) and (c)(ii);
(i) integrating an equation that relates angular parameters of the two-photon fluorescent label, and the light intensity ratio calculated for two-photon fluorescence to determine pairs of angular parameter values that satisfy the two-photon fluorescence equation;
(j) integrating an equation that relates angular parameters of the second harmonic (SH)-active, sum frequency (SF)-active, or difference frequency (DF)-active label, and the light intensity ratio calculated for the second harmonic (SH), sum frequency (SF), or difference frequency (DF) light to determine pairs of angular parameter values that satisfy the second harmonic (SH), sum frequency (SF), or difference frequency equation; and
(k) determining the intersection of the pairs of angular parameter values identified in steps (i) and (j) to determine a unique pair of angular parameter values that satisfy both the two-photon fluorescence and the second harmonic (SH), sum frequency (SF), or difference frequency equations.

27. (canceled)

28. The method of claim 1, wherein the angular parameters comprise a mean tilt angle, an orientational distribution width, or a pairwise combination thereof.

29. A method for detecting a conformational change in a biomolecule, the method comprising:
a) attaching the biomolecule to a planar surface in an oriented manner, wherein the biomolecule is labeled with a two-photon fluorescent label;
b) illuminating the attached biomolecule with excitation light of a first fundamental frequency using a first polarization and a second polarization;
c) detecting a first physical property of light and a second physical property of light generated by the two-photon fluorescent label as a result of the illumination with the first and second polarizations in step (b);
d) subjecting the attached biomolecule to (i) contact with a known ligand, (ii) contact with a candidate binding partner, or (iii) a change in experimental conditions;
e) illuminating the attached biomolecule with excitation light of the first fundamental frequency using the first polarization and the second polarization;
f) detecting a third physical property of light and a fourth physical property of light generated by the two-photon fluorescent label as a result of the illumination with the first and second polarizations in step (e); and
(f) comparing a ratio of the third and fourth physical properties of light detected in step (f) to a ratio of the first and second physical properties of light detected in step (c), wherein a change in the ratio of physical properties of light indicates that the biomolecule has undergone a conformational change.

30. The method of claim 29, wherein the physical properties of two-photon fluorescent light are detected without the use of a lens using a pinhole detection apparatus having a numerical aperture of between about 0.01 and about 0.2.

31.-32. (canceled)

33. The method of claim 29, wherein the two-photon fluorescent label is also second harmonic, sum frequency, or difference frequency active, and wherein a physical property of second harmonic, sum frequency, or difference frequency light is detected serially or simultaneously with the detection of the physical properties of the two-photon fluorescence.

34. The method of claim 33, wherein the ratios compared in step (f) comprise ratios of the physical properties of two-photon fluorescence to the physical properties of second harmonic, sum frequency, or difference frequency light.

35. (canceled)

36. The method of claim 29, wherein the first and second polarizations comprise s-polarization and p-polarization.

37. The method of claim 29, wherein the biomolecule is a protein molecule.

38. The method of claim 37, wherein the protein molecule is a drug target and the known ligand is a known drug or the candidate binding partners are drug candidates.

39. (canceled)

40. The method of claim 37, wherein the two-photon fluorescent label is attached to the protein molecule at one or more engineered cysteine residues.

41. (canceled)

42. The method of claim 37, wherein the two-photon fluorescent label is a nonlinear-active unnatural amino acid that has been incorporated into the protein molecule.

43. (canceled)

44. The method of claim 29, wherein the excitation light is delivered to the planar surface using total internal reflection.

45. The method of claim 29, wherein the biomolecule is attached to the planar surface by insertion into or tethering to a supported lipid bilayer.

46. A method for screening candidate binding partners to identify binding partners that modulate the conformation of a target molecule, the method comprising:
(a) tethering the target molecule to a substrate surface, wherein the target molecule is labeled with a two-photon fluorescent label that is attached to a part of the target molecule that undergoes a conformational change upon contact with a binding partner, and wherein the tethered target molecule has a net orientation on the substrate surface;
(b) illuminating the tethered target molecule with excitation light of a first fundamental frequency;
(c) detecting a first physical property of light generated by the two-photon fluorescent label to generate a baseline signal;
(d) sequentially and individually contacting the tethered target molecule with the one or more candidate binding partners;
(e) detecting a second physical property of light generated by the two-photon fluorescent label in response to illumination by the excitation light of the first fundamental frequency for each of the one or more candidate binding partners; and
(f) comparing the second physical property for each of the one or more candidate binding partners to the first physical property, wherein a change in value of the second physical property for a given candidate binding partner relative to that of the first physical property indicates that the candidate binding partner modulates the conformation of the target molecule.

47. The method of claim 46, wherein the first and second physical properties of light comprise intensities of the light generated by the two-photon fluorescent label under two different polarizations of the excitation light.

48. (canceled)

49. The method of claim 47, wherein the two-photon fluorescent label is also second harmonic (SH)-active, sum frequency (SF)-active, or difference frequency (DF)-active.

50. The method of claim 49, further comprising the steps of:
(g) simultaneously with or subsequently to performing step (c), detecting a first physical property of light generated by the second harmonic (SH)-active, sum frequency (SF)-active, or difference frequency (DF)-active label upon illumination with excitation light of a second fundamental frequency, wherein the second fundamental frequency may be the same as or different than the first fundamental frequency;
(h) simultaneously with or subsequently to performing step (e), detecting a second physical property of light generated by the second harmonic (SH)-active, sum frequency (SF)-active, or difference frequency (DF)-active label upon illumination with excitation light of the second fundamental frequency; and
(i) comparing the second physical property generated by the second harmonic (SH)-active, sum frequency (SF)-active, or difference frequency (DF)-active label for each of the one or more candidate binding partners to the first physical property generated by the second harmonic (SH)-active, sum frequency (SF)-active, or difference frequency (DF)-active label, wherein a change in value of the second physical property for a given candidate binding partner relative to that of the first physical property further indicates that the candidate binding partner modulates the conformation of the target molecule.

51. The method of claim 50, wherein the first and second physical properties of light comprise the intensities of the light generated by the second harmonic (SH)-active, sum frequency (SF)-active, or difference frequency (DF)-active label under two different polarizations of the excitation light.

52. The method of claim 46, wherein the excitation light is directed to the substrate surface in such a way that it is totally internally reflected from the surface.

53. The method of claim 46, wherein two-photon fluorescence is collected without the use of a collection lens using a pin-hole aperture having a numerical aperture of the pin-hole aperture is between 0.01 and 0.2 that is positioned directly above or below the substrate surface at a point where the excitation light of the first fundamental frequency is incident on the substrate surface.

54.-97. (canceled)
